SEQUENCE LISTING (1 sequence)

SEQ ID NO: 1 - DNA, 51 bp, Homo sapiens
  source 1..51: mol_type = genomic DNA
  variation 26: note = SNP C/T
ttttaggtcactacttagtctttcccgctaatgttatagcttccccctccc

(19) United States
(12) Patent Application Publication
(10) Pub. No.: US 2023/0152324 A1
(43) Pub. Date: May 18, 2023

(54) TUMOR-INTRINSIC NLRP3 INFLAMMASOME SIGNALING PATHWAY AS A GENETIC AND FUNCTIONAL BIOMARKER FOR IMMUNOTHERAPY RESPONSE

(71) Applicant: Duke University, Durham, NC (US)

(72) Inventors: Brent A. Hanks, Durham, NC (US); Balamayooran Theivanthiran, Durham, NC (US)

(21) Appl. No.: 18/055,406

(22) Filed: Nov. 14, 2022

Related U.S. Application Data
(60) Provisional application No. 63/280,270, filed on Nov. 17, 2021.

(51) Int. Cl. G01N 33/574; C12Q 1/6886

(52) U.S. Cl. CPC G01N 33/57488; C12Q 1/6886; C12Q 2600/156; C12Q 2600/106

(57) ABSTRACT

The present disclosure describes methods and markers in the NLRP3-HSP70 axis useful for making treatment decisions regarding cancer.

### CROSS-REFERENCE TO RELATED APPLICATIONS

The present application claims priority to U.S. Provisional Patent Application No. 63/280,270, filed Nov. 17, 2021, the entire contents of which are hereby incorporated by reference.

### STATEMENT REGARDING FEDERALLY SPONSORED RESEARCH

This invention was made with government support under 5R37CA249085 awarded by the National Institute of Health. The government has certain rights in the invention.

### REFERENCE TO AN ELECTRONIC SEQUENCE LISTING

The contents of the electronic sequence listing (155554.00678.xml; Size: 93,609 bytes; and Date of Creation: Nov. 12, 2022) is herein incorporated by reference in its entirety.

### BACKGROUND OF THE INVENTION

The establishment of the pre-metastatic niche has been implicated as a key step in metastatic cancer progression. Several tumor-derived soluble factors (TDSFs) have been described that support various steps involved in establishing the pre-metastatic niche. However, conditions in the primary tumor microenvironment that drive the release of these TDSFs and how this process is regulated have remained less clear. While inflammation has been suggested to be a trigger for both the expression and secretion of TDSFs, the exact underlying molecular mechanism regulating specific TDSFs has not been completely described. In addition, studies addressing how therapeutic interventions may modify the release of TDSFs and ultimately influence the pre-metastatic niche are lacking. In particular, it is not understood how the currently available checkpoint inhibitor immunotherapies impact TDSFs and the development of the pre-metastatic niche.

Prior studies have described the phenomenon of disease hyperprogression in select tumors upon treatment with checkpoint inhibitor immunotherapies. While disease hyperprogression (HPD) has been defined based on various parameters, the term generally describes unexpected rapid disease progression upon administration of an anti-PD-1 checkpoint inhibitor. Although limited to an estimated ˜10% of cancer patients undergoing immunotherapy, HPD represents a devastating complication associated with this treatment modality. Indeed, prior studies in multiple solid tumor types have shown patients with HPD to have a median overall survival of 4.6 months relative to a median OS of 7.6 months in non-HPD patients. This emphasizes the importance of understanding the underlying molecular pathogenesis of this phenomenon as it may allow the identification those tumors more susceptible to this complication and prevent its occurrence. While some studies have identified immune cell populations or potential genes associated with HPD, several questions regarding the molecular mechanisms underlying these observations remain. As a result, there is still debate regarding HPD as a complication attributed to checkpoint inhibitor immunotherapies.

The toll-like receptor-4 (TLR4) signaling pathway has been demonstrated to support the development of the pre-metastatic niche in the lung leading to the establishment of pulmonary metastases in various malignancies. This has included a role for TLR4 in the induction of myeloid cell recruitment to the lung as well as the generation of hyperpermeable regions within the lung vasculature. These roles in the development of the pre-metastatic niche are consistent with prior genetic studies correlating loss-of-function polymorphisms in TLR4 with improved clinical outcome in patients with melanoma metastases. Despite these observations, the exact mechanistic role of TLR4 in supporting metastatic progression for specific tumor types remains to be fully elucidated.

The NOD-, LRR- and pyrin domain-containing protein-3 (NLRP3) inflammasome has been a focus of investigation associated with several inflammatory disorders and has been primarily studied in myeloid cell populations such as macrophages and dendritic cells. Upon activation in myeloid cells, NLRP3 oligomerizes with the apoptosis-associated speck-like protein containing a caspase recruitment domain (ASC) adaptor protein to generate a large molecular assembly that catalyzes caspase-1 activation and the downstream release of the pro-inflammatory cytokines, IL-1β and IL-18. While prior studies have described an association between the NLRP3 inflammasome with cancer invasion and metastasis, mechanistic insight into the role of tumor-intrinsic NLRP3 in these processes has remained limited.

### BRIEF SUMMARY OF THE INVENTION

One aspect of the present disclosure provide a method for treating cancer in a subject selected for responsiveness to the treatment comprising obtaining a biological sample from the subject, b. determining the level or activity of a biomarker in the biological sample, wherein the biomarker comprises markers of activation of the NLRP3-HSP70 axis, comparing the level or activity of the biomarker to a control, classifying the subject for likelihood of clinical response to anti-cancer immunotherapy, wherein the levels of the biomarker correlates with anti-cancer immunotherapy efficacy; and administering anti-cancer immunotherapy to the subject wherein the level of the biomarker indicates the subject is likely to be responsive to the anti-cancer immunotherapy or administering an anti-cancer therapy other than immunotherapy wherein the level of the biomarker indicates the subject is unlikely to be responsive to the anti-cancer immunotherapy. Such methods may be used for determining whether a subject is at risk for not responding to an anti-cancer treatment.

A second aspect of the present disclosure provides a method of treating a subject undergoing anti-cancer immunotherapy, the method comprising, obtaining a biological sample from the subject, determining the level or activity of a biomarker in the biological sample, wherein the biomarker comprises markers of activation of the NLRP3-HSP70 axis, comparing the level or activity of the biomarker to a control and ceasing the administration of the anti-cancer immunotherapy if the level or activity of the biomarker is greater than the control.

Another aspect of the present disclosure provides a method of treating a subject who is refractory or not responding to immune checkpoint inhibitor therapy, the method comprising, obtaining a biological sample from the subject, determining the level or activity of a biomarker in the biological sample, wherein the biomarker comprises markers of activation of the NLRP3-HSP70 axis, comparing the level or activity of the biomarker to a control, administering an anti-cancer immunotherapy treatment to the subject if the level or activity of the biomarker is lower than that of the control in step or not administering an anti-cancer immunotherapy to the subject if the biomarker is higher than the level in the control sample of step.

Another aspect of the present disclosure provides a kit for carrying out any one of the methods described herein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Tumor-intrinsic NLRP3 promotes PMN-MDSC accumulation in distant lung tissues. A. Representative example of frequencies of live⁺CD45⁺CD11b⁺Ly6G⁺Ly6C^{lo}F4/80⁻ PMN-MDSCs in the lungs of tumor-bearing and non-tumor-bearing autochthonous BRAF^{V600E}PTEN^{-/-} mice. B. qrt-PCR analysis of Cxcl5 and Cxcl2 expression by CD45⁺EpCAM⁻ and CD45⁻EpCAM⁺ cell populations derived from the lung tissues of non-tumor-bearing and tumor-bearing BRAF^{V600E}PTEN^{-/-} mice (n=3). Statistical analysis performed by two-way ANOVA followed by Sidak's multiple comparisons test. C. Experimental schematic to investigate the role of tumor NLRP3 on lung PMN-MDSC accumulation. KD, knockdown. NTC, non-target control. D. Flow cytometry analysis of PMN-MDSCs in the lung tissues of non-tumor-bearing, BRAF^{V600E}PTEN^{-/-} tumor-bearing, and BRAF^{V600E}PTEN^{-/-}-NLRP3^{KD} tumor-bearing mice (n=3). Statistical analysis performed by one-way ANOVA followed by Tukey's multiple comparisons test. E. Qrt-PCR analysis of Cxcl1, Cxcl2, Cxcl3, and Cxcl5 expression in FACS-purified CD45⁻ EpCAM⁺ lung epithelial cells derived from BRAF^{V600E}PTEN^{-/-} tumor-bearing and BRAF^{V600E}PTEN^{-/-}-NLRP3^{KD} tumor-bearing mice (n=3). F. Experimental schematic to verify the role of tumor-intrinsic NLRP3 in metastatic progression. G. Flow cytometry analysis of PMN-MDSCs in the lung tissues of BRAF^{V600E}PTEN^{-/-} tumor-bearing mice following treatment with either NLRP3 inhibitor (NLRP3i) or vehicle control (Ctrl, n=4). H. Left, Low-magnification imaging of resected lung tissues following treatment with either NLRP3i or Ctrl. 4×; Scale bars, 2000 μm. Right, Survival curve analysis of BRAF^{V600E}PTEN^{-/-} tumor-bearing mice following treatment with either NLRP3i or Ctrl (n=5). Statistical analysis performed by Log-rank test. All two-group comparisons based on unpaired t tests. All data representative of 2-3 independent experiments and expressed as mean values±SEM (* P<0.05, ** P<0.005, *** P<0.0005).

FIG. 2. HSP70 stimulates a TLR4-Wnt5a signaling axis in lung epithelial tissues to drive PMN-MDSC accumulation. A. HSP70 ELISA analysis of lung tissues harvested from non-tumor-bearing and tumor-bearing BRAF^{V600E}PTEN^{-/-} mice (n=3). B. HSP70 Western blot analysis of lung tissues harvested from non-tumor-bearing and tumor-bearing BRAF^{V600E}PTEN^{-/-} mice (n=4). C. CXCL5 Western blot analysis (Left) and flow cytometry analysis of PMN-MDSCs (Right) in lung tissues following intraperitoneal (i.p.) delivery of normal saline versus recombinant HSP70 (rHSP70) (n=3). D. Top, Wnt5a Western blot analysis of lung tissues harvested from non-tumor-bearing and tumor-bearing BRAF^{V600E}PTEN^{-/-} mice (n=4). Bottom, Wnt5a Western blot analysis of MLE12 lung epithelial cell lysates following HSP70 treatment±TLR4 inhibitor (TLR4i). E. Flow cytometry analysis of PMN-MDSCs in the lung tissues of TLR4^{f/f} (TLR4^{+/+}) and SPC-TLR4^{-/-} (TLR4^{-/-}) transgenic mice (n=3). TAM, tamoxifen. F. Cxcl5 qrt-PCR analysis of CD45⁺EpCAM⁻ and CD45⁻EpCAM⁺ cells FACS-sorted from the lung tissues of TLR4^{f/f} and SPC-TLR4^{-/-} transgenic mice (n=3). Statistical analysis performed by two-way ANOVA followed by Sidak's multiple comparisons test. G. Wnt5a, Cxcl5, Cxcl1, Cxcl2 qrt-PCR analysis of CD45⁻EpCAM⁺ cells FACS-sorted from TLR4^{f/f} and SPC-TLR4^{-/-} transgenic mice following i.p. delivery of normal saline versus rHSP70 (n=3). Statistical analysis performed by two-way ANOVA followed by Tukey's multiple comparisons test. All two-group comparisons based on unpaired t tests. All data representative of 2-3 independent experiments and expressed as mean values±SEM (* P<0.05, *** P<0.0005).

FIG. 3. TLR4-Wnt5a signaling axis in lung epithelial tissues promotes pulmonary metastatic progression. A. Top, primary BRAF^{V600E}PTEN^{-/-} tumor weight measurements following resection from TLR4^{f/f} (TLR4^{+/+}) and SPC-TLR4^{-/-} (TLR4^{-/-}) transgenic mice (n=3). Bottom, Representative TRP2 IHC and TRP2 qrt-PCR analysis of lung tissues resected from TLR4^{f/f} and SPC-TLR4^{-/-} transgenic mice bearing BRAF^{V600E}PTEN^{-/-} tumors (n=3). 40×; Scale bars, 20 μm. B. Left, Representative low-magnification imaging of lungs resected from TLR4^{+/+} and SPC-TLR4^{-/-} transgenic mice following BRAF^{V600E}PTEN^{-/-} tumor tail vein injection. 4×; Scale bars, 2000 μm. Right, Quantification of metastatic tumor burden (n=3). C. Left, Representative S100β IHC of lung tissues resected from TLR4^{+/+} and SPC-TLR4^{-/-} transgenic mice following BRAF^{V600E}PTEN^{-/-} tumor tail vein injection. 40×; Scale bars, 20 μm; Inset, 10×. Right, S100β-positive cells enumerated per lung based on IHC microscopy (n=4). D. Lung weight measurements following resection from TLR4^{+/+} and SPC-TLR4^{-/-} transgenic mice±primary BRAF^{V600E}PTEN^{-/-} tumor (n=4). Statistical analysis performed by two-way ANOVA followed by Sidak's multiple comparisons test. All two-group comparisons based on unpaired t tests. All data representative of 2-3 independent experiments and expressed as mean values±SEM (ns, non-significant. * P<0.05, *** P<0.0005).

FIG. 4. Anti-PD-1 immunotherapy promotes PMN-MDSC accumulation in the lung via the tumor-intrinsic NLRP3-HSP70 axis. A. Flow cytometry analysis of PMN-MDSCs in the lung tissues of BRAF^{V600E}PTEN^{-/-} tumor-bearing mice following treatment with either IgG isotype control (IgG Ctrl) or anti-PD-1 antibody (α-PD-1) (n=3). Statistical analysis performed by unpaired t test. B. Representative cytological analysis of the bronchoalveolar fluid (BALF) isolated from BRAF^{V600E}PTEN^{-/-} tumor-bearing mice following treatment with either IgG Ctrl or α-PD-1. Red arrows, PMN morphology. 40×; Scale bars=20 μm; Inset, 100×. C. Wnt5a and Cxcl5 qrt-PCR analysis of CD45⁺EpCAM⁻ and CD45⁻EpCAM⁺ cells isolated by FACS from lung tissues of BRAF^{V600E}PTEN^{-/-} transgenic mice following treatment with either IgG Ctrl or α-PD-1 (n=3). Statistical analysis performed by two-way ANOVA followed by Tukey's multiple comparisons test. D. Wnt5a and Cxcl5 qrt-PCR analysis of CD45⁺EpCAM⁻ and CD45⁻EpCAM⁺ cells isolated by FACS from lung tissues of mice harboring either control BRAF^{V600E}PTEN^{-/-} tumors (Vector Ctrl) or BRAF^{V600E}PTEN^{-/-}HSP70^{-/-} tumors (Hsp70^{-/-})±anti-PD-1 treatment (n=3). Statistical analysis performed by two-way ANOVA followed by Tukey's multiple comparisons test. E. Left, Flow cytometry analysis of PMN-MDSCs in the BALF of BRAF^{V600E}PTEN^{-/-} tumor-bearing mice following treatment with either IgG Ctrl, α-PD-1, or α-PD-1+anti-HSP70 antibody (α-PD1/α-HSP70) (n=3). Right, Representative Ly6G IHC of lung tissues harvested from BRAF^{V600E}PTEN^{-/-} tumor-bearing mice following treatment with IgG Ctrl, α-PD1, or α-PD1/HSP70. 40×; Scale bars, 20 μm. Statistical analysis performed by one-way ANOVA followed by Tukey's multiple comparisons test. F. Flow cytometry analysis of PMN-MDSCs in the lung tissues harvested from BRAF^{V600E}PTEN^{-/-} tumor-bearing mice following treatment with IgG Ctrl, α-PD-1, or α-PD1/NLRP3i (n=3). Statistical analysis performed by two-way ANOVA followed by Tukey's multiple comparisons test. All data representative of 2-3 independent experiments and expressed as mean values±SEM (ns, non-significant. * P<0.05, ** P<0.005).

FIG. 5. Tumor-intrinsic NLRP3-HPS70 axis promotes disease hyperprogression in response to anti-PD-1 immunotherapy. A. BRAF^{V600E}CDKN2A^{-/-}PTEN^{-/-} tumor volume and weight measurements following treatment of BRAF^{V600E}PTEN^{-/-} tumors with IgG Ctrl (pre-IgG Ctrl) or -α-PD1 (pre-α-PD1) (n=3). B. Quantification of Ly6G and CD8 IHC of primary BRAF^{V600E}CDKN2A^{-/-}PTEN^{-/-} tumors following experiment in A (n=3). C. Representative images and quantification of S100β IHC of lung tissues derived from transgenic BRAF^{V600E}PTEN^{-/-} mice following IgG Ctrl or α-PD1±Ly6G antibody ablation (n=5). 20×; Scale bar=50 μm. D. Representative hematoxylin and eosin (H&E) microscopy (10×; Scale bar=100 μm), Ly6G immunofluorescence (20×; Scale bar=50 μm), and S100β IHC (20×; Scale bar=50 μm. Inset, 40×; Scale bar=20 μm) of lungs derived from transgenic BRAF^{V600E}PTEN^{-/-} mice following IgG Ctrl, α-PD1, or α-PD1/NLRP3i therapy. E. Top, quantitation of S100β IHC in lung tissue from experiment described in D (n=3-4). Bottom, TRP2 qrt-PCR analysis of the lung tissues from experiment described in D (n=3). F. Representative images and quantification of S100β IHC of lung tissues derived from transgenic BRAF^{V600E}PTEN^{-/-} mice following IgG Ctrl, α-PD1, or α-PD1/α-HSP70 (n=4). 20×; Scale bar=50 μm. C, E, F. Statistical analysis performed by one-way ANOVA followed by Tukey's multiple comparisons test. All two-group comparisons based on unpaired t tests. All data representative of 2-3 independent experiments and expressed as mean values±SEM (* P<0.05, ** P<0.005, *** P<0.0005).

FIG. 6. Genetic amplification of NLRP3 promotes disease hyperprogression in response to anti-PD-1 immunotherapy. A. Incidence of NLRP3 amplification in human tumor types based on the TCGA. Data visualized using cBioPortal. B. Flow cytometry analysis of PMN-MDSCs in the lungs of Ctrl and NLRP3a tumor-bearing mice (n=3). C. Left, S100β IHC of lungs derived from Ctrl and NLRP3a tumor-bearing mice. 20×; Scale bar=50 μm. Red arrows, S100β-positive cells. Right, Quantification of S100β⁺ cells in the lungs of Ctrl and NLRP3a tumor-bearing mice (n=3). D. Left, Flow cytometry analysis of lung tissues resected from TLR4^{+/+} control vs SPC-TLR4^{-/-} mice harboring control BRAF^{V600E}PTEN^{-/-} tumors or BRAF^{V600E}PTEN^{-/-}-NLRP3a tumors (n=3). Right, Wnt5a qrt-PCR analysis of lung tissues of TLR4^{+/+} control vs SPC-TLR4^{-/-} mice harboring control BRAF^{V600E}PTEN^{-/-} tumors or BRAF^{V600E}PTEN^{-/-}-NLRP3a tumors (n=3). E. ELISA analysis of plasma HSP70 levels in Ctrl and NLRP3a tumor-bearing mice following IgG Ctrl and α-PD1 (n=3). Statistical analysis performed by two-way ANOVA followed by Tukey's multiple comparisons test. F. Flow cytometry analysis of PMN-MDSCs in the lungs of Ctrl and NLRP3a tumor-bearing mice following α-PD1 (n=3). G. Ctrl and NLRP3a tumor growth rates following IgG Ctrl and α-PD1. Expressed as a ratio of tumor growth rate during α-PD1 therapy relative to IgG Ctrl. All two-group comparisons based on unpaired t tests. All data representative of 2-3 independent experiments and expressed as mean values±SEM (* P<0.05, *** P<0.0005).

FIG. 7. The HSP70-TLR4 signaling axis in lung epithelial tissues promotes primary tumor progression and anti-PD-1 immunotherapy resistance. A. Control BRAF^{V600E}PTEN^{-/-} tumor growth rates in TLR4^{+/+} and SPC-TLR4^{-/-} mice during IgG Ctrl vs α-PD1. Expressed as a ratio of tumor growth rate during α-PD1 therapy relative to IgG Ctrl (n=6). B. Flow cytometry analysis of PMN-MDSCs in the blood of BRAF^{V600E}PTEN^{-/-} tumor-bearing TLR4^{+/+} and SPC-TLR4^{-/-} mice (n=5). C. Representative flow cytometry dot plot of PD-1 expression by circulating PMN-MDSCs in TLR4^{+/+} and SPC-TLR4^{-/-} transgenic mice (n=5 mice). D. G-CSF qrt-PCR analysis of CD45⁺EpCAM⁻ and CD45⁻EpCAM⁺ cells FACS sorted from the lungs of non-tumor-bearing and BRAF^{V600E}PTEN^{-/-} tumor-bearing mice (n=3). Statistical analysis performed by two-way ANOVA followed by Tukey's multiple comparisons test. E. G-CSF qrt-PCR analysis of lung tissues harvested from TLR4^{+/+} and SPC-TLR4^{-/-} mice (n=3). F. Representative G-CSF IHC of lung tissues harvested from TLR4^{+/+} and SPC-TLR4^{-/-} mice (n=3). 20×; Scale bar=50 μm. G. G-CSF ELISA analysis of the plasma of TLR4^{+/+} and SPC-TLR4^{-/-} transgenic mice (n=3). H. Left, G-CSF ELISA analysis of the plasma of transgenic BRAF^{V600E}PTEN^{-/-} mice following α-PD1±HSP70i (n=4). Right, G-CSF qrt-PCR analysis of the lung tissues of transgenic BRAF^{V600E}PTEN^{-/-} mice following α-PD1±HSP70i (n=4). I. Representative G-CSF Western blot analysis of MLE12 lung epithelial cells following treatment with recombinant Wnt5a. All two-group comparisons based on unpaired t tests. All data representative of 2-3 independent experiments and expressed as mean values±SEM (* P<0.05, ** P<0.005, *** P<0.0005).

FIG. 8. Activation of the tumor-intrinsic NLRP3-HSP70 axis is associated with hyperprogression in melanoma patients undergoing anti-PD-1 immunotherapy. A. Baseline plasma HSP70 ELISA measurements in advanced melanoma patients prior to initiating anti-PD-1 immunotherapy (n=29). HPD, hyperprogression. B. NLRP3-ASC proximity ligation assay (PLA) analysis of baseline tumor tissues in advanced melanoma patients prior to initiating anti-PD-1 immunotherapy (n=34). PD, progressive disease. SD, stable disease. PR, partial response. CR, complete response. Red dots, represent NLRP3-ASC interactions. 40×; Scale bar=5 μm. C. Progression-free survival analysis (PFS) of advanced melanoma patients stratified according to baseline tumor NLRP3-ASC PLA scores. Low NLRP3, below the NLRP3-ASC PLA median. High NLRP3, equal to or above the NLRP3-ASC PLA median. Statistical analysis performed by log-rank test. D. Schematic of the tumor-intrinsic NLRP3-HSP70 signaling axis and its role in promoting metastatic progression in the lung in response to α-PD1. Generated using BioRender. A, B. Statistical analysis performed by one-way ANOVA followed by Tukey's multiple comparisons test. All data representative of 2 independent experiments and expressed as mean values±SEM (* P<0.05, *** P<0.0005).

FIG. 9. CD45+CD11b+Ly6G+Ly6CloF4/80− PMN-MDSCs in tumor-bearing mice suppress CD8+ T Cell activity in vitro and in vivo. A. Representative flow cytometry histogram of CFSE dilution assay measuring CD8+ T cell proliferation in response to anti-CD3/anti-CD28 beads±PMN-MDSCs (n=3). B. In vitro splenic CD8+ T cell proliferation assay following in vivo ablation of PMN-MDSCs with Ly6G antibody (α-Ly6G) vs IgG isotype control (Ctrl). Left, representative flow dot plot after Ctrl vs α-Ly6G treatment. Right, Flow cytometry CellTracer dilution assay performed on day 3 and day 6 of culture. Flow cytometry plots representative of two independent experiments.

FIG. 10. Supportive data for initial lung epithelial cell and lung PMN-MDSC studies. A. Gating strategy for FACS isolation of CD45+EpCAM− and CD45-EpCAM+ cell populations from mouse lung tissues. B. Schematic describing the previously reported PD-L1:NLRP3:HSP70:TLR4:Wnt5a:CXCL5 signaling cascade that promotes adaptive resistance to anti-PD-1 immunotherapy. C. Flow cytometry analysis of PMN-MDSCs and CD8+ T cells in the lung tissues of NLRP3^{-/-} mice treated with vehicle control (Ctrl) versus NLRP3i (n=4). Statistical analysis performed by unpaired t test. Data representative of 2 independent experiments and expressed as mean values±* P<0.05).

FIG. 11. Role of host myeloid NLRP3 inflammasome activity in primary tumor progression, lung PMN-MDSC accumulation, and lung metastatic progression. A. Lung PMN-MDSC flow cytometry analysis of BRAF^{V600E}PTEN^{-/-} melanoma-bearing wild type and NLRP3^{-/-} hosts (n=4). B. BRAF^{V600E}PTEN^{-/-} melanoma progression in wild type versus NLRP3^{-/-} hosts (n=5). C. Melanoma antigen TRP2 qrt-PCR analysis of lung tissues resected from wild type and NLRP3^{-/-} hosts. Statistical analysis performed by unpaired t test. All data representative of 2 independent experiments and expressed as mean values±SEM. Ns, non-significant.

FIG. 12. Melanoma NLRP3 activation induces the upregulation of HSP70 over IL-1β. A. IL-1β qrt-PCR analysis of FACS-sorted CD45 and CD45 cell populations from BRAF PTEN melanomas on day 7 and day 30 of tumor growth (n=3). B. Representative Western blot analysis of the active form of cleaved IL-1β (17 kDa) and HSP70 in human melanoma cell line conditioned media±LPS or LPS/ATP. TCL, total cell lysate. SNT, supernatant. C. IL-1β and HSP70 ELISA analysis of WM266 human melanoma cell line conditioned media±LPS/ATP (n=2). D. Left, HSP70 ELISA analysis of BRAF^{V600E}PTEN^{-/-} melanoma cell-conditioned media under various conditions (n=3). Right, HSP70 ELISA analysis of splenic DC-conditioned media under various conditions (n=3). E. Left IL-1β ELISA analysis of BRAF^{V600E}PTEN^{-/-} melanoma cell-conditioned media under various conditions (n=2). Right, IL-1β ELISA analysis of splenic DC-conditioned media under various conditions (n=3). All data representative of 2 independent experiments and is expressed as mean values±SEM.

FIG. 13. Generation of SPC-Cre-ER^{T2}/TLR4^{fl/fl} transgenic mice and a TRP2-specific qrt-PCR assay for the detection of melanoma metastases in the lung. A. Flow cytometry analysis of PMN-MDSCs in the lung tissues of non-tumor-bearing mice following intra-nasal or intra-peritoneal (ip) delivery of recombinant HSP70 versus sterile saline (n=3). B. Representative flow cytometry plot of TLR4 expression by lung epithelial cells in SPC-Cre-ER^{T2} transgenic mice following 4HT or sterile saline i.p. delivery. Tlr4, Wnt5a, and Cxcl5 qrt-PCR expression analysis of FACS sorted CD45-EpCAM+ cells derived from the lungs of SPC-Cre-ERT2 transgenic mice following 4HT or sterile saline ip delivery (n=3). C. Flow cytometry analysis of CD8+ T cells in SPC-Cre-ER^{T2} transgenic mice following 4HT or sterile saline ip delivery. (n=3). D. Characterization of the TRP2 qrt-PCR lung metastasis assay using SYBR green versus TaqMan probes. Statistical analysis performed by unpaired t test. All data representative of 2 independent experiments and expressed as mean values±SEM (* P<0.05, ** P<0.005, ** 5).

FIG. 14. Lung PMN-MDSC and metastases studies for BRAF^{V600E}CDKN2A^{-/-}PTEN^{-/-} (YUMM1) melanoma-bearing mice. A. Representative Western blot analysis of HSP70 and Wnt5a in BRAF^{V600E}PTEN^{-/-} and YUMM1 cell lines±IFNγ or IFNγ/α-PD-L1. B. Flow cytometry analysis of PMN-MDSCs in lung tissues harvested from BRAF^{V600E}CDKN2A^{-/-}PTEN^{-/-} tumor-bearing mice (n=3). C. H&E microscopy (top, 10×; Scale bar=100 μm) and S100! IHC (bottom, 40×; Scale bar=20 μm) of lung tissues derived from YUMM1 tumor-bearing TLR4^{+/+} and SPC-TLR4^{-/-} transgenic mice (n=3). Statistical analysis performed by unpaired t test. All data representative of 2 independent experiments and expressed as mean values±SEM (* P<0.05).

FIG. 15. Lung PMN-MDSC and metastases studies for E0771 breast cancer-bearing mice. A. Top, Representative Western blot analysis of cleaved Caspase-1 and Wnt5a in E0771 breast cancer cell line±IFNγ or IFNγ/α-PD-L1 or IFNγ/α-PD-L1/NLRP3i. Bottom, Representative Western blot comparing NLPR3 expression levels between E0771 cell line vs control BRAF^{V600E}PTEN^{-/-} cell line vs NLRP3 amplified BRAF^{V600E}PTEN^{-/-} cell line. B. Flow cytometry analysis of PMN-MDSCs in BALF derived from E0771 tumor-bearing TLR4^{+/+} and SPC-TLR4^{-/-} transgenic mice (n=4). BALF, bronchoalveolar lavage fluid. C. H&E microscopy of lung tissues derived from E0771 tumor-bearing TLR4^{+/+} and SPC-TLR4^{-/-} transgenic mice. 10×; Scale bar=100 μm. D. Low-magnification imaging of lungs resected from E0771 tumor-bearing TLR4^{+/+} and SPC-TLR4^{-/-} transgenic mice. 4×; Scale bar=2000 μm; inset, 40×; Scale bar=200 μm (n=4). All data representative of 2 independent experiments and expressed as mean values±SEM (* P<0.05).

FIG. 16. The tumor-intrinsic NLRP3 inflammasome drives the accumulation of VEGFR1⁺ HPCs in the lung via lung epithelial TLR4 activation. A. Flow cytometry analysis of CD45+CD34+CD133+ckit+VEGFR1+ HPCs in the lungs of BRAF^{V600E}PTEN^{-/-}-bearing and non-BRAF^{V600E}PTEN^{-/-}-bearing mice (n=3). B. Flow cytometry analysis of CD45+CD34+CD133+ckit+VEGFR1+ HPCs in the lungs of SPC-TLR4^{-/-} and wild type control mice bearing BRAF^{V600E}PTEN^{-/-} melanomas (n=4). C. Flow cytometry analysis of CD45+CD34+CD133+ckit+VEGFR1+ HPCs in the lungs of mice harboring BRAF^{V600E}PTEN^{-/-}-NLRP3a and BRAF^{V600E}PTEN^{-/-}-Ctrl tumors (n=3). D. Flow cytometry analysis of CD45+CD34+CD133+ckit+VEGFR1+ HPCs in the lungs of BRAF^{V600E}PTEN^{-/-} bearing mice on day 7 and day 20 post-resection (n=4). E. Flow cytometry analysis of PMN-MDSCs in the lungs of BRAF^{V600E}PTEN^{-/-}-bearing mice on day 7 and day 20 post-resection (n=3). Statistical analysis performed by unpaired t test. All data is expressed as mean values±SEM (* P<0.05, ** P<0.005).

FIG. 17. Tumor-dependent fibronectin expression in the lung ECM is supported by the TLR4-Wnt5a axis. A. qrt-PCR analysis of fibronectin expression in the lungs of non-tumor-bearing and tumor-bearing TLR4^{+/+} control versus SPC-TLR4^{-/-} hosts. Statistical analysis performed by one-way ANOVA followed by Tukey's multiple comparisons test. Right, Fibronectin IHC (red) of lung tissues resected from tumor-bearing TLR4^{+/+} control versus SPC-TLR4^{-/-} mice. 20×; Scale bar=60 μm. B. Fibronectin IHC (red) of lung tissues resected from anti-PD-1 treated versus anti-PD-1/Wnti treated tumor-bearing mice. Wnti, PORCN inhibitor=ETC159. 20×; Scale bar=50 μm. C. qrt-PCR analysis of Tgfr2, Spp1, and S100a4 expression levels in the lungs of TLR4^{+/+} control and SPC-TLR4^{-/-} hosts. Statistical analysis performed by unpaired t test. All data representative of 2 independent experiments and expressed as mean values±SEM. (* P<0.05, ** P<0.005, *** P<0.0005).

FIG. 18. Induction of PMN-MDSC recruitment by anti-PD-1 immunotherapy can be reversed by the inhibition of HSP70 and NLRP3. A. Flow cytometry analysis of PMN-MDSCs in lung tissues derived from BRAF^{V600E}PTEN^{-/-} tumor-bearing and non-tumor-bearing mice (n=4). B. CXCL5 qrt-PCR analysis of lung tissues derived from BRAF^{V600E}PTEN^{-/-} tumor-bearing and non-tumor-bearing mice (n=3). C. Representative H&E microscopy of lung tissues derived from BRAF^{V600E}PTEN^{-/-} tumor-bearing mice following IgG Ctrl, α-PD-1, and α-PD-1/HSP70i therapy (n=3). 40×; Scale bars=20 μm D. Left, tumor volume measurements during IgG Ctrl, α-PD-1, HSP70i, and α-PD-1/HSP70i therapy (n=6). Right, Flow cytometry analysis of PMN-MDSCs and CD8+ T cells in resected tumors following IgG Ctrl, α-PD-1, HSP70i, and α-PD-1/HSP70i therapy (n=3-4). Statistical analysis performed by one-way ANOVA followed by Tukey's multiple comparisons test. E. Flow cytometry analysis of PMN-MDSCs in BALF of BRAF^{V600E}PTEN^{-/-} tumor-bearing and non-tumor-bearing mice following IgG Ctrl, α-PD-1, and α-PD-1/NLRP3i therapy (n=3). A,B,E. Statistical analysis performed by two-way ANOVA followed by Tukey's multiple comparisons test. All data representative of 2 independent experiments and expressed as mean values±SEM (* P<0.05, ** P<0.005***, P<0.0005).

FIG. 19. Anti-PD-1 immunotherapy induces Wnt5a upregulation in lung epithelial tissues in a NLRP3-dependent manner. A. Wnt5a qrt-PCR analysis of lung-derived CD45-EpCAM+ cells following IgG Ctrl and α-PD-1 therapy (n=3). B. Representative Wnt5a Western blot analysis of lung tissues derived from BRAF^{V600E}PTEN^{-/-} tumor-bearing mice following IgG Ctrl, α-PD-1, or α-PD-1/NLRP3i therapy. C. Flow cytometry analysis of PMN-MDSCs in lung tissues derived from BRAF^{V600E}PTEN^{-/-} tumor-bearing mice following IgG Ctrl, α-PD-1, or α-PD-1/Wnti therapy (n=3). D. Flow cytometry analysis of PMN-MDSCs in lung tissues derived BRAF^{V600E}PTEN^{-/-} tumor-bearing TLR4^{+/+} and SPC-TLR4^{-/-} transgenic mice following IgG Ctrl or α-PD-1 (n=3-4). Statistical analysis performed by one-way ANOVA followed by Tukey's multiple comparisons test. All data representative of 2 independent experiments and expressed as mean values±SEM (* P<0.05, ** P<0.005).

FIG. 20. Prior Anti-PD-1 immunotherapy enhances PMN-MDSC accumulation and diminishes CD8⁺ T cell infiltration in subsequent tumors. A. Representative Ly6G and CD8 IHC analysis of BRAF^{V600E}CDKN2A^{-/-}PTEN^{-/-} tumors following prior anti-PD-1 versus IgG Ctrl therapy of BRAF^{V600E}PTEN^{-/-} tumors. 20×; Scale bar=50 μm. B. Representative TRP2 IHC of lung tissues derived from transgenic BRAF^{V600E}PTEN^{-/-} mice following IgG Ctrl, α-PD-1, or α-PD-1/HSP70i therapy. 20×; Scale bar=50 μm. All data representative of 2 independent experiments.

FIG. 21. Genetic amplification of Nlrp3 promotes tumor progression and enhances PMN-MDSC recruitment in response to anti-PD-1 immunotherapy. A. Representative NLRP3, Wnt5a, and HSP70 Western blot analysis of a control BRAF^{V600E}PTEN^{-/-} tumor cell line (Ctrl) versus a Nlrp3 amplified BRAF^{V600E}PTEN^{-/-} tumor cell line (NLRP3a). SNT, supernatant. B. Left, Tumor volume measurements of control BRAF^{V600E}PTEN^{-/-} tumors versus NLRP3-amplified BRAF^{V600E}PTEN^{-/-} tumors (NLRP3a) in syngeneic hosts (n=5). Right, Control BRAF^{V600E}PTEN^{-/-} tumor cell line versus BRAF^{V600E}PTEN^{-/-}-NLRP3a tumor cell line proliferation rates based on an in vitro MTT assay (n=3). C. Transwell Matrigel cell invasion assays performed on control BRAF^{V600E}PTEN^{-/-} versus BRAF^{V600E}PTEN^{-/-}-NLRP3a tumor cell lines (n=10). Scale bar=200 μm. D. Flow cytometry analysis of PMN-MDSCs and CD8+ T cells in control BRAF^{V600E}PTEN^{-/-} tumors versus BRAF^{V600E}PTEN^{-/-}-NLRP3a tumors (n=3). E. Plasma IL-1β ELISA analysis of syngeneic mice harboring control BRAF^{V600E}PTEN^{-/-} versus BRAF^{V600E}PTEN^{-/-}-NLRP3a tumors following IgG Ctrl versus α-PD-1 therapy (n=2). ns, non-significant. F. Flow cytometry analysis of PMN-MDSCs and CD8+ T cells in tumor and lung tissues derived from mice harboring control BRAF^{V600E}PTEN^{-/-} tumors versus BRAF^{V600E}PTEN^{-/-}-NLRP3a tumors following IgG Ctrl versus α-PD-1 therapy (n=3). Statistical analysis performed by unpaired t tests. All data representative of 2 independent experiments and expressed as mean values±SEM (* P<0.05).

FIG. 22. Anti-PD-1 immunotherapy enhances progression of the syngeneic E0771 breast cancer model. A. Tumor volume measurements of primary E0771 tumors in syngeneic mice undergoing treatment with anti-PD-1 immunotherapy vs IgG isotype control (n=5). B. PMN-MDSC and CD8+ T cell flow cytometry analysis of harvested E0771 tumors following anti-PD-1 immunotherapy vs IgG isotype control (n=3). Statistical analysis performed by unpaired t tests. C. Representative H&E microscopy of lung tissues resected from E0771-tumor-bearing wild type and SPC-TLR4^{-/-} mice following either IgG isotype control versus anti-PD-1 immunotherapy. 10×; Scale bar=100 μm. 8-10 fields randomly selected per lung for quantification. All data representative of 2 independent experiments and expressed as mean values±SEM (* P<0.05).

FIG. 23. Anti-PD-1 immunotherapy enhances tumor NLRP3 upregulation in a CD8⁺ T cell- and IFN-γ-dependent manner. A. Representative NLRP3 IHC of BRAF^{V600E}PTEN^{-/-} tumors following IgG Ctrl versus α-PD-1 therapy. 20×; Scale bar=50 μm; inset, 40×; Scale bar=20 μm. B. Top, Representative NLRP3 Western blot analysis of resected BRAF^{V600E}PTEN^{-/-} tumor tissues following IgG Ctrl versus α-PD-1 therapy. Bottom, Representative NLRP3 Western blot analysis of resected BRAF^{V600E}PTEN^{-/-} tumor tissues following IgG Ctrl versus α-PD-1 therapy±CD8 antibody ablation. C. Representative NLRP3 Western blot analysis of BRAF^{V600E}PTEN^{-/-}-OVA tumor cells following co-incubation with OT-1 CD8+ T cells±anti-PD-1 antibody anti-IFNγ antibody. All data representative of 2-3 independent experiments.

FIG. 24. TLR4 signaling in the lung epithelium promotes tumor progression and anti-PD-1 resistance in the E0771 breast cancer model. A. Tumor volume measurements of primary E0771 tumors in syngeneic SPC-TLR4^{-/-} hosts relative to TLR4^{+/+} control hosts (n=5). B. Flow cytometry analysis of circulating PD-1+CD11b+Ly6G+Ly6C^{lo}F4/80− PMN-MDSCs (n=3). C. Flow cytometry analysis of PMN-MDSCs and CD8+ T cells in E0771 tumors in syngeneic SPC-TLR4^{-/-} hosts relative to TLR4^{+/+} control hosts (n=3). D. Arg1 qrt-PCR analysis of FACS-sorted circulating PD-1+CD11b+Ly6G+Ly6C^{lo}F4/80− PMN-MDSCs (n=3). Statistical analysis performed by unpaired t tests. All data representative of 2 independent experiments and expressed as mean values±SEM (* P<0.05).

FIG. 25. Anti-PD-1 immunotherapy promotes the accumulation of circulating PMN-MDSCs in the blood. A. Flow cytometry analysis of PMN-MDSCs in the blood of mice harboring BRAF^{V600E}PTEN^{-/-} tumors following IgG Ctrl versus α-PD-1 therapy (n=3). Statistical analysis performed by two-way ANOVA followed by Tukey's multiple comparisons test. Data representative of 2 independent experiments and expressed as mean values±SEM (* P<0.05). B. Schematic illustrating the NLRP3:HSP70:TLR4:Wnt5a:CXCL5/G-CSF signaling cascade that drives pre-metastatic niche development and metastatic progression in response to α-PD-1 therapy.

FIG. 26. Supportive clinical biomarker data for the NLRP3 pathway in melanoma patients. A. Baseline plasma IL-1β ELISA analysis in advanced melanoma patients undergoing α-PD-1 therapy (n=29). HPD, hyperprogressive disease. ns, non-significant. Statistical analysis performed by one-way ANOVA followed by Tukey's multiple comparisons test. B. Overall survival analysis of advanced melanoma patients undergoing α-PD-1 therapy based on NLRP3-ASC PLA assay performed on baseline tumor tissue specimens (n=34). Low NLRP3, below the NLRP3-ASC PLA median. High NLRP3, equal to or above the NLRP3-ASC PLA median. Statistical analysis performed by log-rank test (* P<0.05).

### DETAILED DESCRIPTION OF THE INVENTION

NLRP3 inflammasome has been shown to be involved in tumorigenesis. Germline genetic studies show NLRP3 is associated with elevated plasma HSP70 levels in advanced melanoma patients as well as inferior progress-free survival while undergoing immunotherapy. Importantly, the genetic profile of NLRP3 in a tumor can determine tumor response to anti-PD-1 immunotherapy. As shown herein, baseline markers of the tumor-intrinsic NLRP3-HSP70 signaling pathway correlate with resistance and disease hyperprogression in patients undergoing anti-PD-1 immunotherapy.

Some embodiments of the present disclosure provide a method for treating cancer in a subject selected for responsiveness to the treatment comprising obtaining a biological sample from the subject, b. determining the level or activity of a biomarker in the biological sample, wherein the biomarker comprises markers of activation of the NLRP3-HSP70 axis, comparing the level or activity of the biomarker to a control, classifying the subject for likelihood of clinical response to anti-cancer immunotherapy, wherein the levels of the biomarker correlates with anti-cancer immunotherapy efficacy; and administering anti-cancer immunotherapy to the subject wherein the level of the biomarker indicates the subject is likely to be responsive to the anti-cancer immunotherapy or administering an anti-cancer therapy other than immunotherapy wherein the level of the biomarker indicates the subject is unlikely to be responsive to the anti-cancer immunotherapy. Such methods may be used for determining whether a subject is at risk for not responding to an anti-cancer treatment.

In various embodiments, the present methods direct a clinical decision regarding whether a patient is to receive a specific treatment. In one embodiment, the present methods are predictive of a response to an anti-cancer immunotherapy. In various embodiments, the present invention directs the treatment of a cancer patient, including, for example, what type of treatment should be administered or withheld.

As used herein, the terms “anti-cancer immunotherapy”, “immune blockade therapy”, checkpoint inhibitor therapy” are used interchangeable and refers to those forms of cancer immunotherapies that target immune checkpoints, the key regulators of the immune system that when stimulated can dampen the immune response to an immunologic stimulus. Examples of such therapies include, but are not limited to, the use of anti-CTLA4, anti-PD-1, anti-PD-L1 and the like.

As is known in the art, a cancer is generally considered as uncontrolled cell growth. The methods of the present invention can be used to treat any cancer, and any metastases thereof, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma. In some embodiments, the cancer comprises melanoma, breast cancer, renal cell carcinoma, non-small cell lung cancer, colorectal cancer, Merkel cell carcinoma, gastroesophageal cancer, gastric cancer or pancreatic cancer.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

For example, treating cancer in a subject includes the reducing, repressing, delaying or preventing cancer growth, reduction of tumor volume, and/or preventing, repressing, delaying or reducing metastasis of the tumor. Treating cancer in a subject also includes the reduction of the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; and (d) reducing or ameliorating at least one symptom of cancer. In some embodiments, the optimum effective amount can be readily determined by one skilled in the art using routine experimentation. In some embodiments, the treatment is immunotherapy. As used herein, immunotherapy is treatment that uses a person's own immune system to fight cancer. Immunotherapy can boost or change how the immune system works so it can find and attack cancer cells. Immunotherapy may also use substances made by the body or in a laboratory to boost the immune system and help the body find and destroy cancer cells. Immunotherapy can be used alone or in combination with other cancer treatments. Immunotherapy may also be called immune therapy and may include immune checkpoint therapy or immune blockade therapy. In some embodiments, the immunotherapy comprises a PD-1 inhibitor or a PD-L1 inhibitor. In some embodiments, the therapy may comprise an NLRP3 inhibitor.

A “subject in need thereof” as utilized herein may refer to a subject in need of treatment for a disease or disorder associated with a suspected tumor, such as a tumor. A subject in need thereof may include a subject having a cancer that is characterized by gross abnormality visible by X-ray, computerized tomography (CT), or magnetic resonance imaging (MM), but which has not been diagnosed as a tumor by histology or immunofluorescence. In some embodiments, the tumor comprises a skin cancer. In some embodiments, the tumor comprises melanoma. In some embodiments, the tumor comprises a primary tumor and one or more tumors in secondary sites. In some embodiments the secondary site are metastatic sites. In some embodiments, the tumor is a melanoma and the metastatic site is the lymph node, brain, bone, liver or lungs.

The term “subject” may be used interchangeably with the terms “individual” and “patient” and includes human and non-human mammalian subjects.

Some embodiments of the preset disclosure provide a method of determining whether a subject is at risk for developing disease hyperprogression when undergoing treatment with an anti-cancer immunotherapy. Hyperprogression the accelerated, or more rapid than expected, growth or progression of a cancer after treatment is initiated. Hyperprogression is a tumor response in which the existing underlying tumor grows rapidly after initiating treatment, wherein the treatment is typically an immune checkpoint inhibitor.

Some embodiments of the preset disclosure provide methods of treating a subject who is refractory or not responding to immune checkpoint inhibitor therapy. These subjects may not respond to immune checkpoint therapy or may develop a secondary resistance to the immune therapy over time.

Some embodiments of the present disclosure provide a method of determining whether a subject is at risk of not responding to an anti-cancer immunotherapy treatment, at risk for developing disease hyperprogression or a subject who is refractory or not responding to immune checkpoint inhibitor therapy, the method comprising obtaining a biological same from the subject. The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample may be obtained directly from a subject (e.g., by blood or tissue sampling) or from a third party (e.g., received from an intermediary, such as a healthcare provider or lab technician).

Some embodiments of the present disclosure provide a method of determining whether a subject is at risk of not responding to an anti-cancer immunotherapy treatment, at risk for developing disease hyperprogression or a subject who is refractory or not responding to immune checkpoint inhibitor therapy, the method comprising obtaining a biological same from the subject and determining the level or activity of a biomarker in the biological sample, wherein the biomarker comprises markers detecting activation of the NLRP3-HSP70 axis. Determining the level or activity of a biomarker in the sample includes, but is not limited to, measuring the amount or expression of biomarker protein, DNA or RNA. Techniques to measure protein, DNA and RNA are known in the art. In addition to the amount or expression, a biomarker may also be measured by a functional or other such property such as its activity, binding, solubility, size, weight, denaturation, amphoteric nature, optical activity, charge, sequence or reactive properties. Markers detecting activation of the NLRP3-HSP70 axis, include but are not limited to HSP70, NLRP3, Wnt5a, TLR4, CXCR2

In particular embodiments, the biomarker is HSP70. HSP70 is a Heat shock protein (HSP) which are expressed in response to various biological stresses, including high temperatures. There are several major families of HSPs that include HSP70, there are HSP90 and HSP100. The HSP70 family is a set of highly conserved proteins that are induced by a variety of biological stresses, including heat stress, in every organism in which the proteins have been examined. The human HSP70 family members include: HSP70, a protein which is strongly inducible in all organisms but which is also constitutively expressed in primate cells. In conjunction with other heat shock proteins, HSP70 stabilizes existing proteins against aggregation and mediates the folding of newly translated proteins in the cytosol and organelles. HSP70 is also involved in the ubiquitin-proteasome pathway through interaction with the AU-rich element RNA-binding protein 1.

The inventors have found elevated levels of HSP70 in the plasma of melanoma patients undergoing anti-PD-1 immunotherapy to be associated with resistance to this treatment modality. Accordingly, one aspect of the present disclosure provides a method of determining whether a subject is at risk of not responding to an anti-cancer immunotherapy treatment based on HSP70 as a biomarker.

Additional germline genetic studies performed by the inventors focused on a single nucleotide polymorphism (SNP) of NLRP3. These studies have demonstrated the affect allele of this SNP to be associated with both elevated plasma HSP70 levels in advanced melanoma patients as well as in inferior progress-free survival while undergoing anti-PD-1 immunotherapy. The NLRP3 polymorphism is SNP ID: rs12239046 Polymorphism: C/T, Transition Substitution Context Sequence: TTTTAGGTCACTACTTAGTCTTTCC[C/T]GCTAATGTTATAGCTTCCCCCTCCC (SEQ ID NO: 1). Many genetic alterations involving both NLRP3 itself as well as several of its regulators have been identified and associated with both inflammatory conditions as well as various malignancies. Based on the data provided herein and gathered by the inventors, it is believed that the genetic profile of the NLRP3 inflammasome pathway in any given tumor can determine whether a tumor does or does not respond to anti-PD-1 immunotherapy. Since there can be multiple genetic alterations in numerous regulators of this pathway that ultimately impact the signaling activity of the NLRP3 inflammasome. Embodiments of the present disclosure further comprise functional activity assays capable of measuring the NLRP3 activation state in tumor tissues (for both germline and somatic mutations) as well as in peripheral blood mononuclear cells (germline only). The later assay would be blood-based and significantly more practical for clinical use. We expect for patients demonstrating either elevated NLRP3 activity based on this test and/or to possess genetic alterations in this signaling pathway predicted to enhance it's signaling activity to be less likely to respond to anti-PD-1 immunotherapy.

Additionally, the data provided herein shows that this same pathway promotes the accumulation of granulocytic myeloid-derived suppressor cells in distant tissues as well, thus establishing a pre-metastatic niche that promotes metastatic progression in distant organs. In line with these findings, the inventors have also found patients demonstrating evidence of disease hyperprogression in response to anti-PD-1 immunotherapy to also have a significant elevation in the baseline levels of plasma HSP70. Thus, other embodiments of the present disclosure provide that elevated NLRP3 activation scores based on the methods provided herein and/or genetic alterations involving this signaling pathway that are predicted to enhance its activation, indicate that a particular patient would be at increased risk for developing disease hyperprogression when undergoing treatment with anti-PD-1 therapy.

Biomarkers may be measured by any means known in the art. These include, but are not limited to polymerase chain reaction, reverse transcriptase polymerase chain reaction, quantitative reverse transcriptase polymerase chain reaction, Western blot, sequencing, southern blot, northern blot, enzyme linked immunosorbent assay, immunostaining, proximity ligation assay and/or fluorescent in-situ hybridization. A proximity ligation assay may be performed with NLRP3/NALP3 and apoptosis-associated speck-like protein containing a caspase recruitment domain (ASC) as targets. In some embodiments, the biomarker may be evaluated or measured using Q-RT-PCR, Western blot, RNA sequencing, proteomic studies, sequencing, fluorescent in situ hybridization (FISH), ELISA, immunostaining, or proximal ligation assay.

Some embodiments of the present disclosure provide a method of determining whether a subject is at risk of not responding to an anti-cancer immunotherapy treatment, at risk for developing disease hyperprogression or a subject who is refractory or not responding to immune checkpoint inhibitor therapy, the method comprising obtaining a biological same from the subject and determining the level or activity of a biomarker in the biological sample, wherein the biomarker is HSP70 and comparing the level of activity of the biomarker to a control. A control may be a subject who is not undergoing treatment or evaluation for the disease the subject is need it. A control may be a healthy subject, who does not have a diagnosis of cancer, or healthy tissue.

Some embodiments of the present disclosure provide a method of determining whether a subject is at risk of not responding to an anti-cancer immunotherapy treatment, at risk for developing disease hyperprogression or a subject who is refractory or not responding to immune checkpoint inhibitor therapy, the method comprising obtaining a biological same from the subject and determining the level or activity of a biomarker in the biological sample, wherein the biomarker is HSP70, comparing the level of activity of the biomarker to a control and administering an anti-cancer immunotherapy treatment to the subject if the level or activity of the biomarker is lower than that of the control in or not administering an anti-cancer immunotherapy to the subject if the level of the biomarker is higher than the level in the control sample. An anti-cancer immunotherapy can be any known anti-cancer treatment immunotherapy known in the art. In some embodiments, the anti-cancer immunotherapy comprises immune blockade therapy or an immune checkpoint inhibitor. In some embodiments, the anti-cancer immunotherapy comprises a PD-1 inhibitor or a PD-L1 inhibitor.

Some embodiments of the present disclosure provide a method of determining whether a subject is at risk of not responding to an anti-cancer immunotherapy treatment, at risk for developing disease hyperprogression or a subject who is refractory or not responding to immune checkpoint inhibitor therapy, the method comprising obtaining a biological same from the subject and determining the level or activity of a biomarker in the biological sample, wherein the biomarker is HSP70, comparing the level of activity of the biomarker to a control and administering an anti-cancer immunotherapy treatment to the subject based on the biomarker. In some embodiments, the method further comprises administering an NLRP3 inhibitor. Examples of a NLRP3 inhibitors include, but are not limited to antibodies, small molecules, peptides, miRNAs, siRNAs, oligonucleotides, cytokines or agonists. NLRP3 inhibitors further include, but are not limited to pembrolizumab, Z-VAD-FMK, MCC950, Resveratrol, Arglabin, CB2R agonist, miRNA-223, beta-hydroxybutyrate (BHB), Type I interferon (IFN-beta), JC124, CY09, dapansutrile (OLT1177).

The anti-cancer therapies described herein may be combined with other known therapies as necessary to treat the subject in need.

Another aspect of the present disclosure provides a kit for the prognosis, diagnosis, or prediction of a subject's response to anti-cancer immunotherapy comprising, consisting or, or consisting essentially of: (a) a means for analyzing the biomarker, wherein the biomarker comprises HSP70; (b) a control; and (c) instructions for use.

In some embodiments, the kits may comprise a plurality of primers or probes, primary or secondary antibodies, oligonucleotides, templates, a negative control, positive control, nucleotides, amplification medium, preservatives or buffers to detect or measure the biomarker.

In some embodiments, the kit includes a packaging material. As used herein, the term “packaging material” can refer to a physical structure housing the components of the kit. In some instances, the packaging material maintains sterility of the kit components, and is made of material commonly used for such purposes (e.g., paper, corrugated fiber, glass, plastic, foil, ampules, etc.). Other materials useful in the performance of the assays are included in the kits, including test tubes, transfer pipettes, and the like. In some cases, the kits also include written instructions for the use of one or more of these reagents in any of the assays described herein.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

### Example 1—Tumor-intrinsic NLRP3-HSP70-TLR4 Axis Drives Pre-Metastatic Niche Development and Hyperprogression During Anti-PD-1 Immunotherapy

Reference is made to the manuscript: Theivanthiran et al., “Tumor-intrinsic NLRP3-HSP70-TLR4 Axis Drives Pre-Metastatic Niche Development and Hyperprogression During Anti-PD-1 Immunotherapy,” the content of which is incorporated herein by reference in its entirety.

Tumor-Intrinsic NLRP3 Drives PMN-MDSC Accumulation in Distant Tissues.

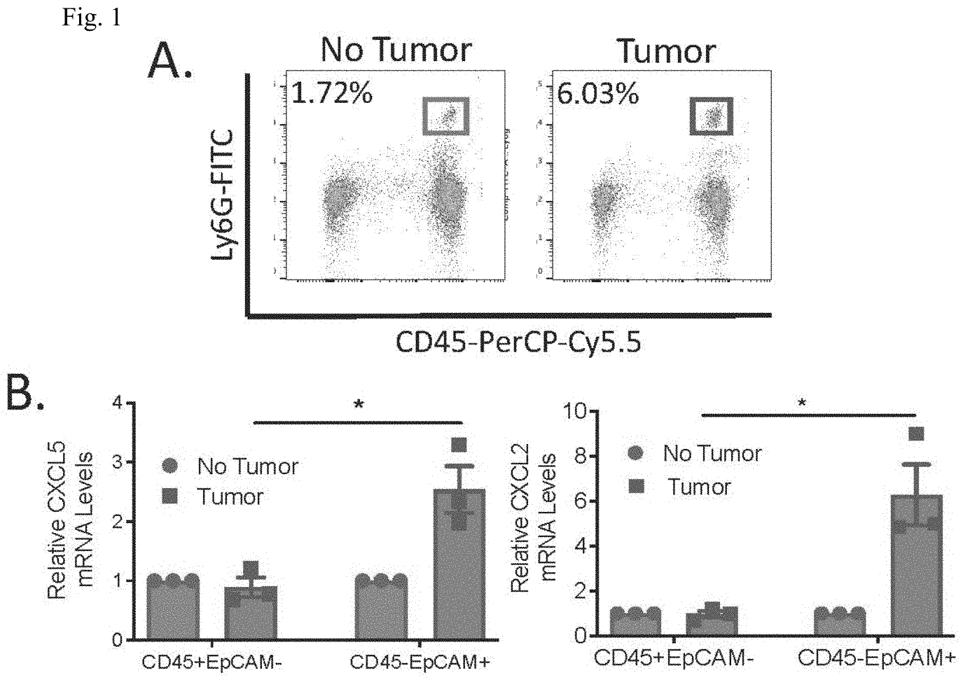

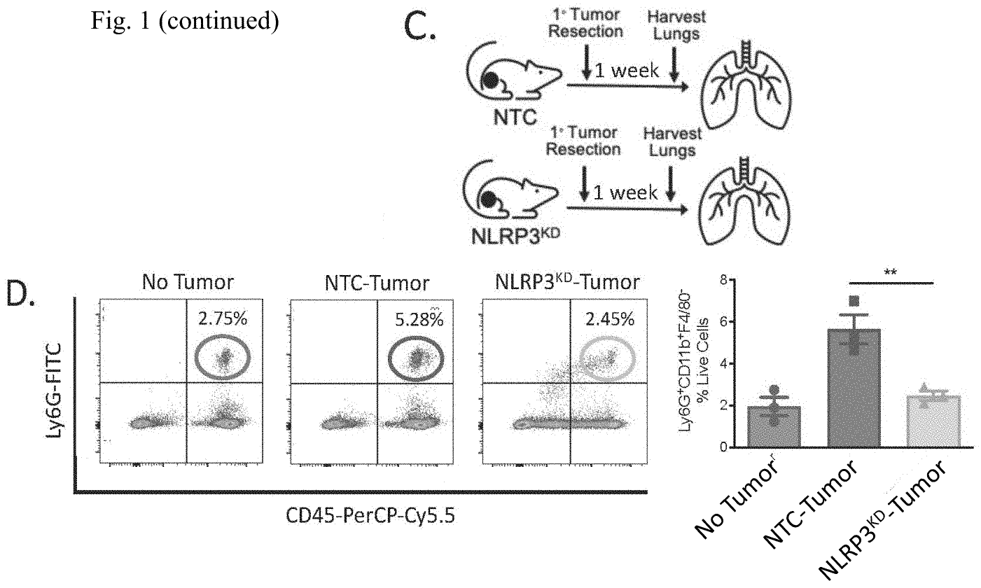

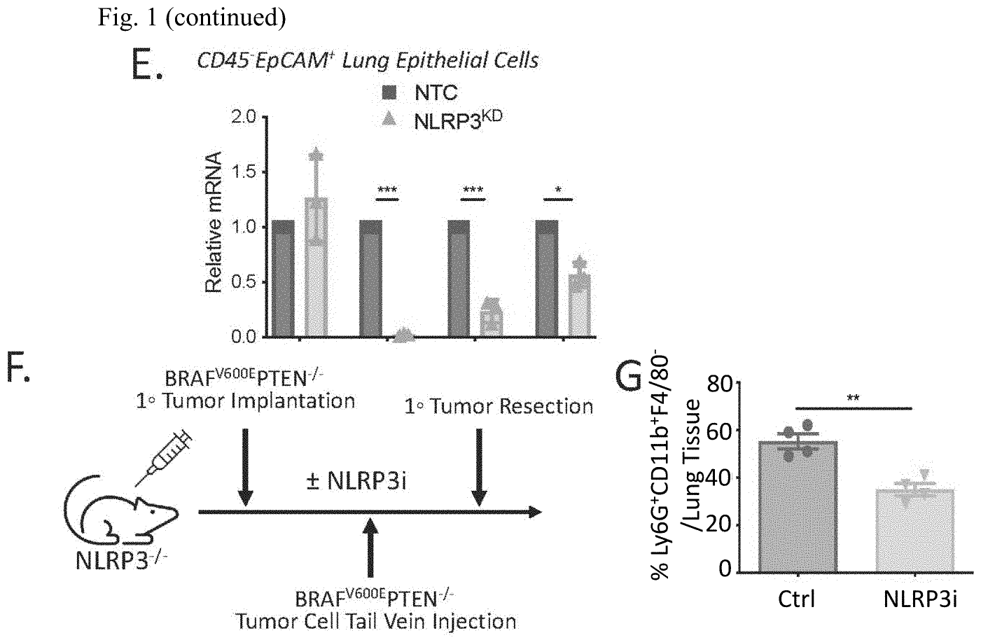

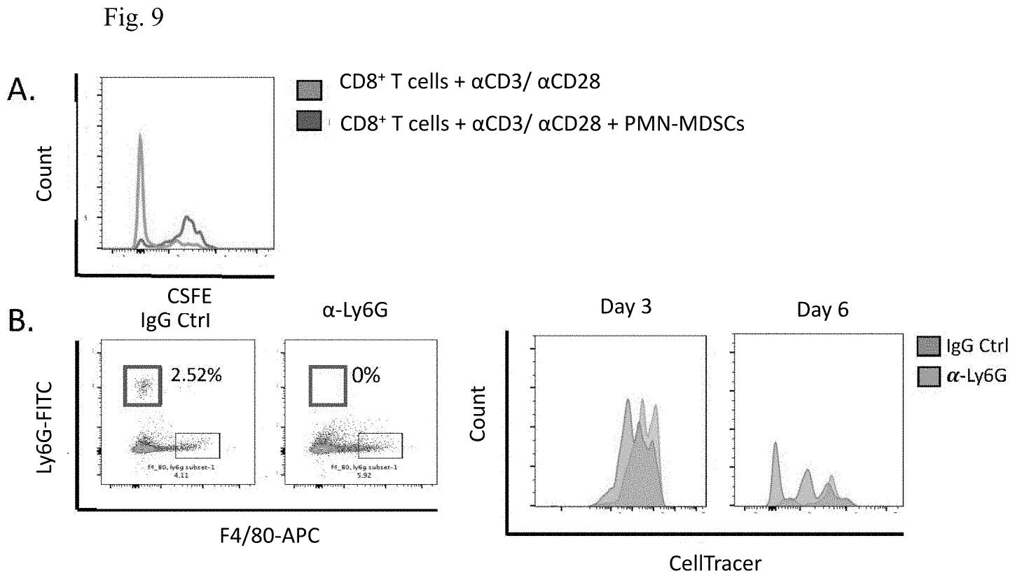

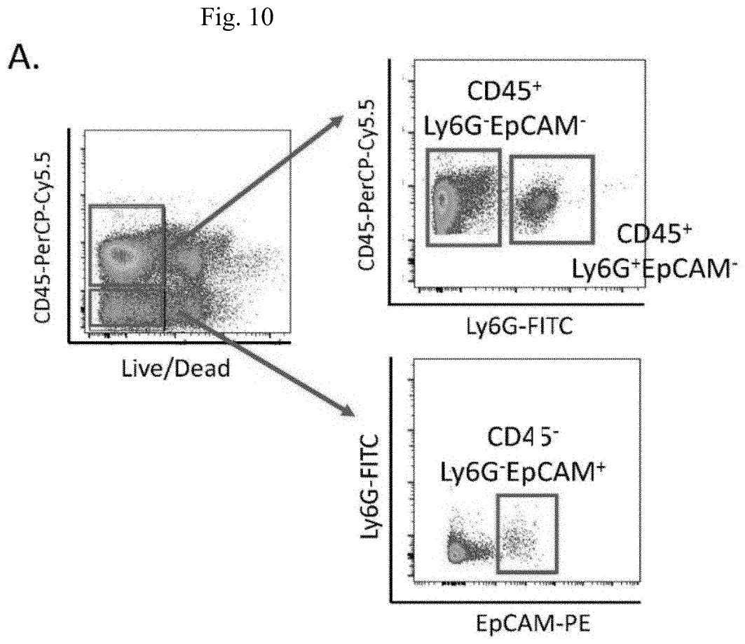

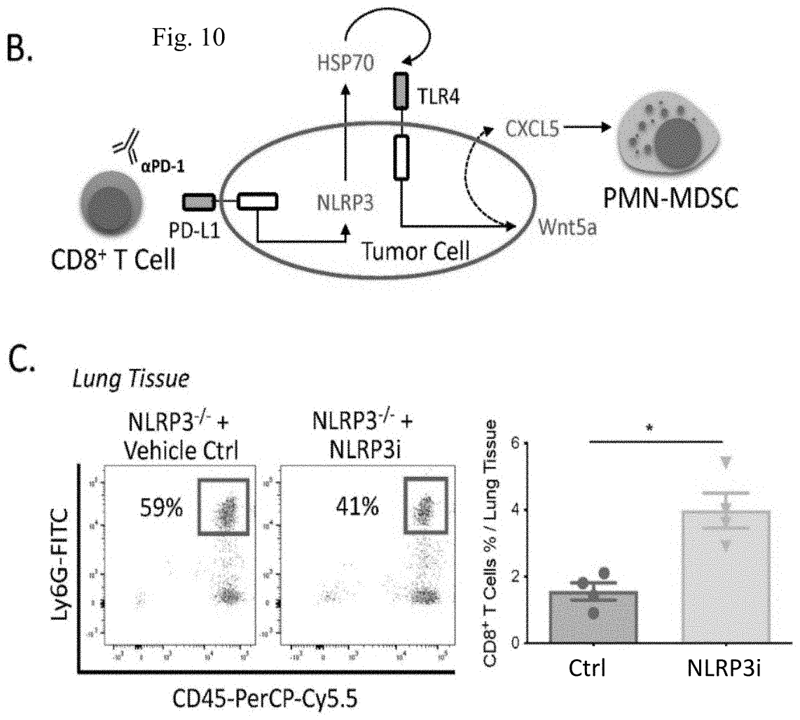

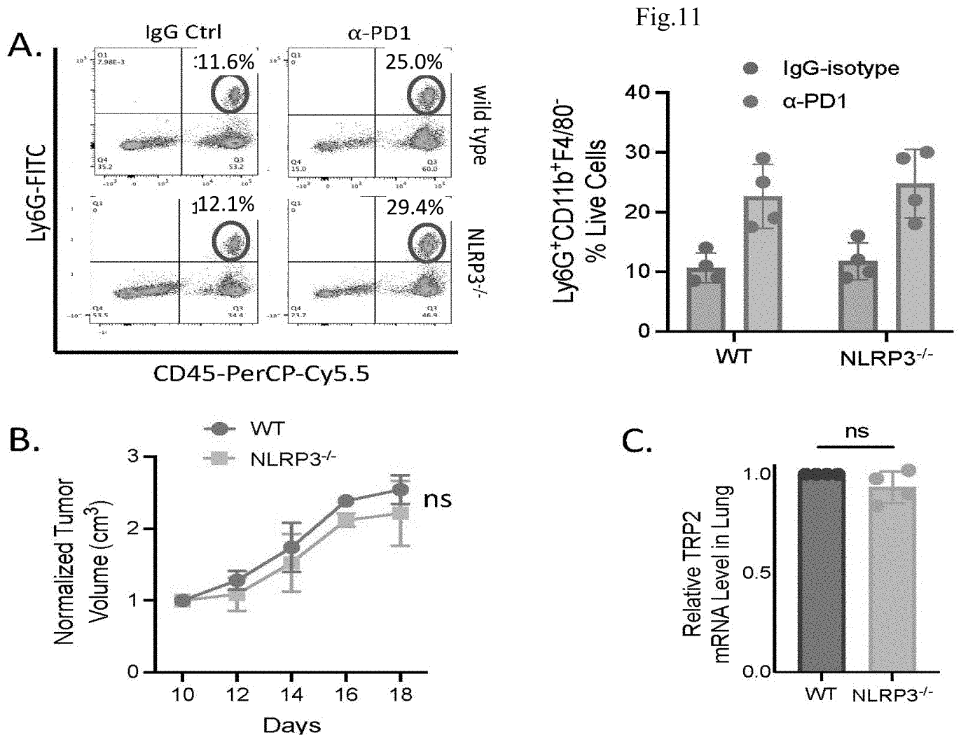

Prior studies have described elevated numbers of circulating neutrophils in tumor-bearing mice relative to healthy controls (22). Consistent with these findings, we also identified a significant increase in a CD45⁺CD11b⁺Ly6G⁺Ly6C^{lo}F4/80⁻ cell population in the lung tissues of transgenic BRAF^{V600E}PTEN^{-/-} mice harboring primary melanomas relative to those BRAF^{V600E}PTEN^{-/-} mice with no active disease (FIG. 1A). Herein, we refer to this population as granulocytic myeloid-derived suppressor cells (PMN-MDSCs) as we have shown these cells to suppress CD8⁺ T cell proliferation in vitro and in vivo while also diminishing responses to anti-PD-1 immunotherapy and supporting tumor progression in vivo (FIG. 9) (19, 23). Using quantitative real-time polymerase chain reaction (qrt-PCR) analysis of sorted cell populations derived from the harvested lung tissues of BRAF^{V600E}PTEN^{-/-} mice, we further found that the C-X-C motif chemokine receptor 2 (CXCR2)-dependent chemokines, Cxcl5 and Cxcl2, were upregulated by CD45⁻EpCAM⁺ lung epithelial cells only in tumor-bearing hosts (FIG. 1B, and FIG. 10A). Our previous work showed that the recruitment of PMN-MDSCs into primary tumor tissues was dependent upon activation of the tumor-intrinsic NLRP3 inflammasome inducing a HSP70-TLR4-Wnt5a-CXCL5 signaling cascade (FIG. 10B) (19). Given these prior findings, we performed syngeneic tumor experiments to evaluate the impact of genetically silencing NLRP3 in a BRAF^{V600E}PTEN^{-/-} melanoma cell line (BRAF^{V600E}PTEN^{-/-}-NLRP3^{KD}) on the accumulation of PMN-MDSCs in lung tissues (FIG. 1C)(19). Primary tumors were resected prior to metastatic progression and lung tissues were harvested for flow cytometry analysis one week later. This study showed that silencing NLRP3 expression in primary tumors led to a reduction in distant lung PMN-MDSCs to levels comparable to non-tumor-bearing mice (FIG. 1D). We were then interested in determining whether the NLRP3 inflammasome expressed by primary melanomas regulated gene expression in distant tissues. Therefore, single-cell suspensions were generated from harvested lung tissues and sorted by fluorescence activated cell sorting (FACS) to quantitate the expression of CXCR2-dependent chemokines in CD45⁻EpCAM⁺ lung epithelial cells by qrt-PCR (FIG. 10A). Consistent with our prior findings, genetic silencing of the tumor-expressed NLRP3 inflammasome significantly suppressed Cxcl2, Cxcl3, and Cxcl5 expression in these remote epithelial tissues (FIG. 1E). NLRP3 inflammasome activity in myeloid cells has been implicated in several inflammatory diseases and may contribute to this observed phenomenon (24, 25). To formally exclude a role for myeloid NLRP3 inflammasome activity, additional experiments were performed in NLRP3^{-/-} mice harboring primary BRAF^{V600E}PTEN^{-/-} melanomas that were further subjected to the delivery of BRAF^{V600E}PTEN^{-/-} melanoma cells via tail vein injection. These mice were treated with the pharmacologic NLRP3 inhibitor, MCC950, versus a vehicle control (FIG. 1F). Overall, these studies demonstrated tumor NLRP3 inhibition to suppress accumulation of PMN-MDSCs in the lungs, enhance lung CD8⁺ T cell trafficking, inhibit the establishment of pulmonary metastases, and prolong the survival of these NLRP3^{-/-} mice (FIGS. 1, G and H, and FIG. 10C). These findings are further supported by additional experiments demonstrating no differences in primary BRAF^{V600E}PTEN^{-/-} melanoma growth, lung PMN-MDSC accumulation, or distant metastatic progression in NLRP3^{-/-} versus wild type mice (FIG. 11). Together, these data indicate that the tumor-intrinsic NLRP3 inflammasome, rather than the host myeloid NLRP3 inflammasome, promotes the accumulation of PMN-MDSCs in distant lung tissues.

HSP70 Triggers a TLR4-Wnt5a Signaling Axis in Lung Epithelial Tissues to Promote PMN-MDSC Accumulation in the Lung

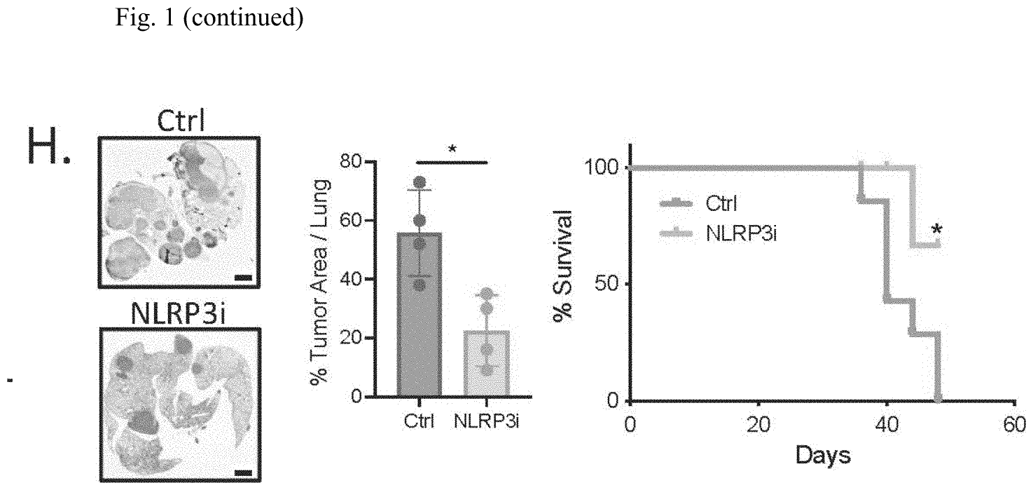

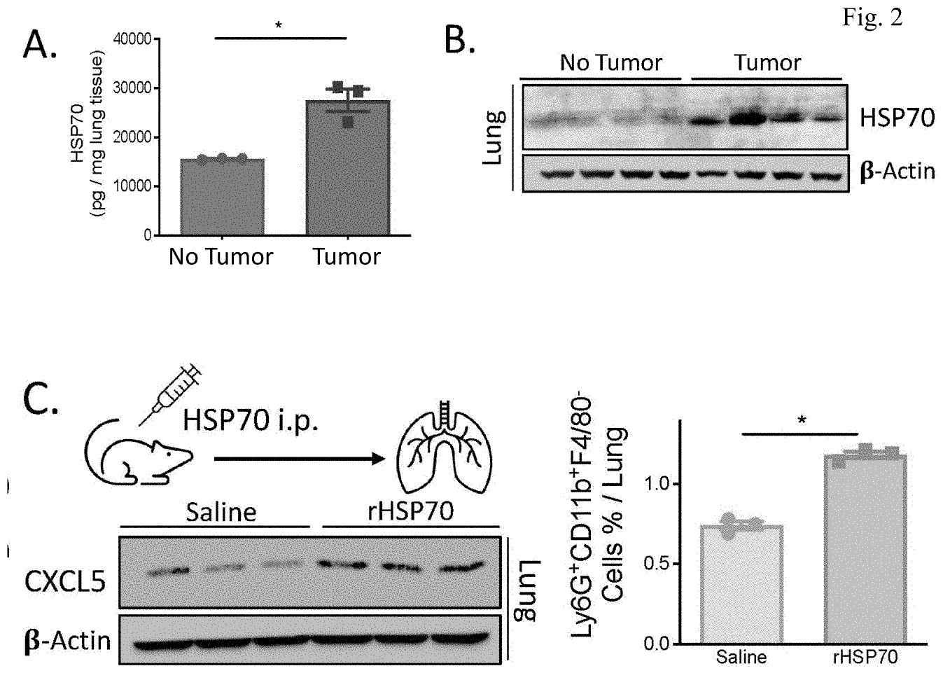

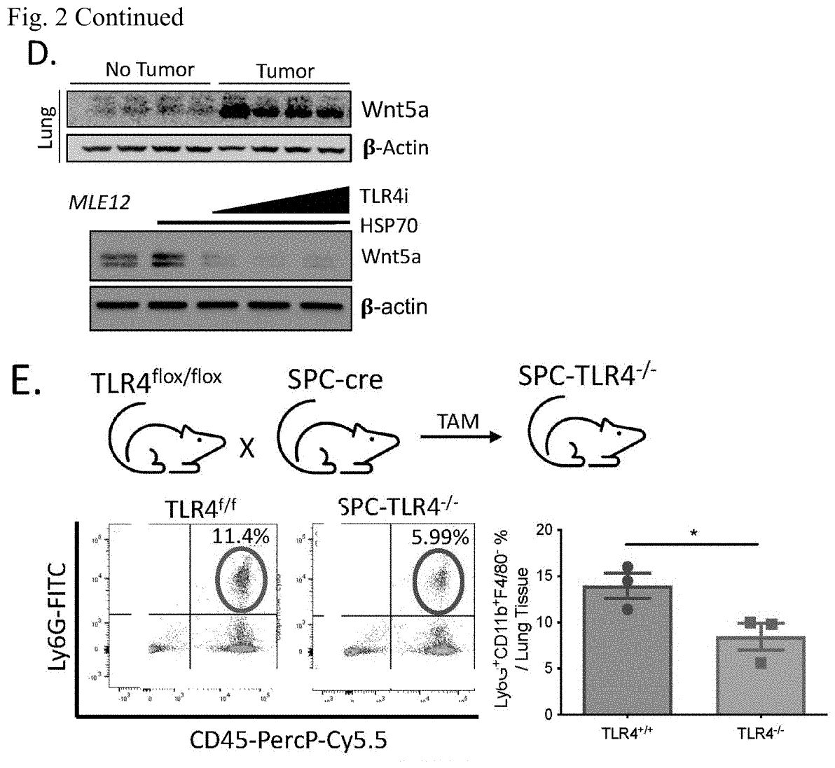

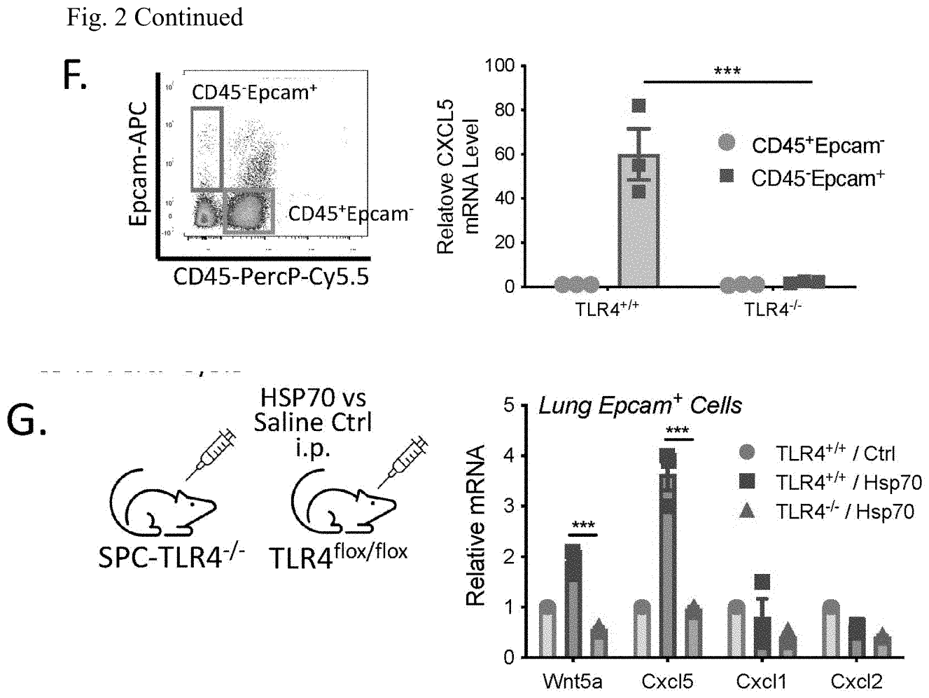

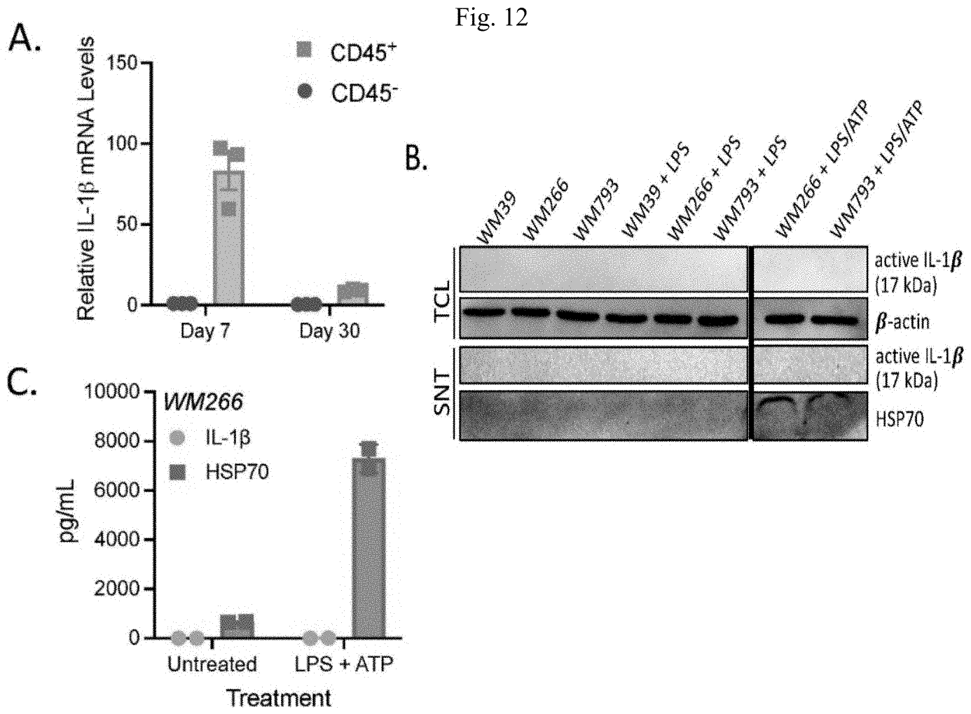

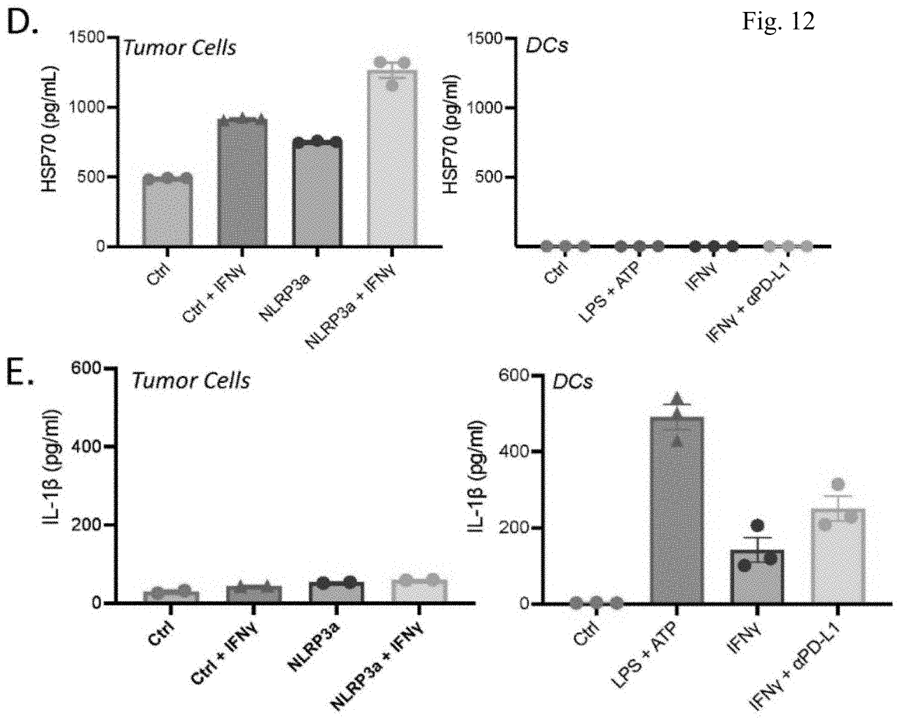

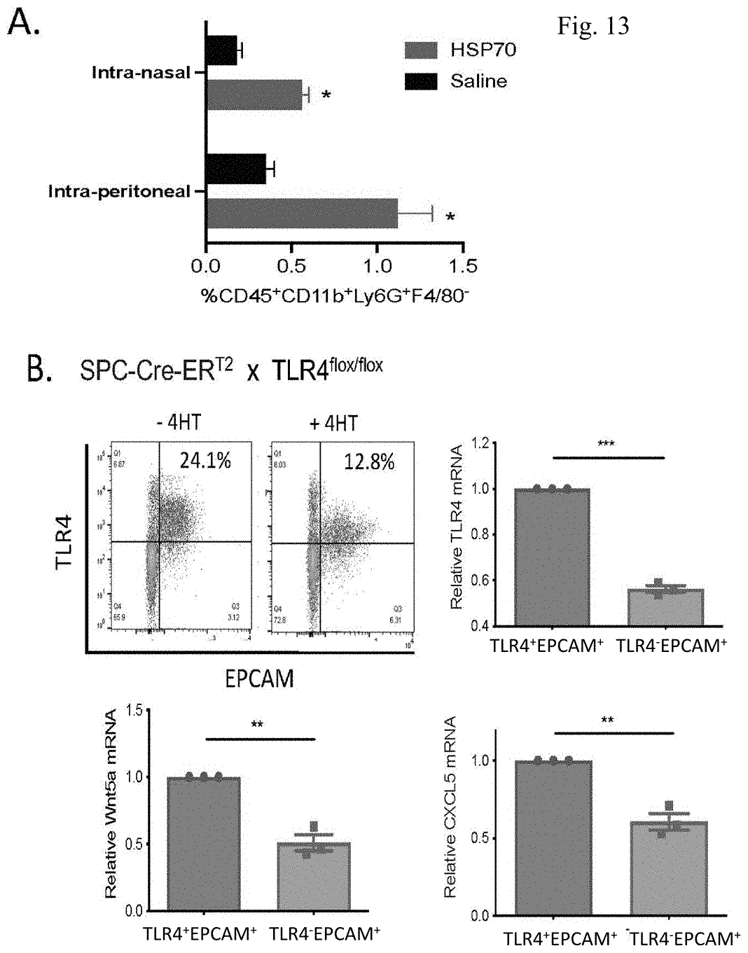

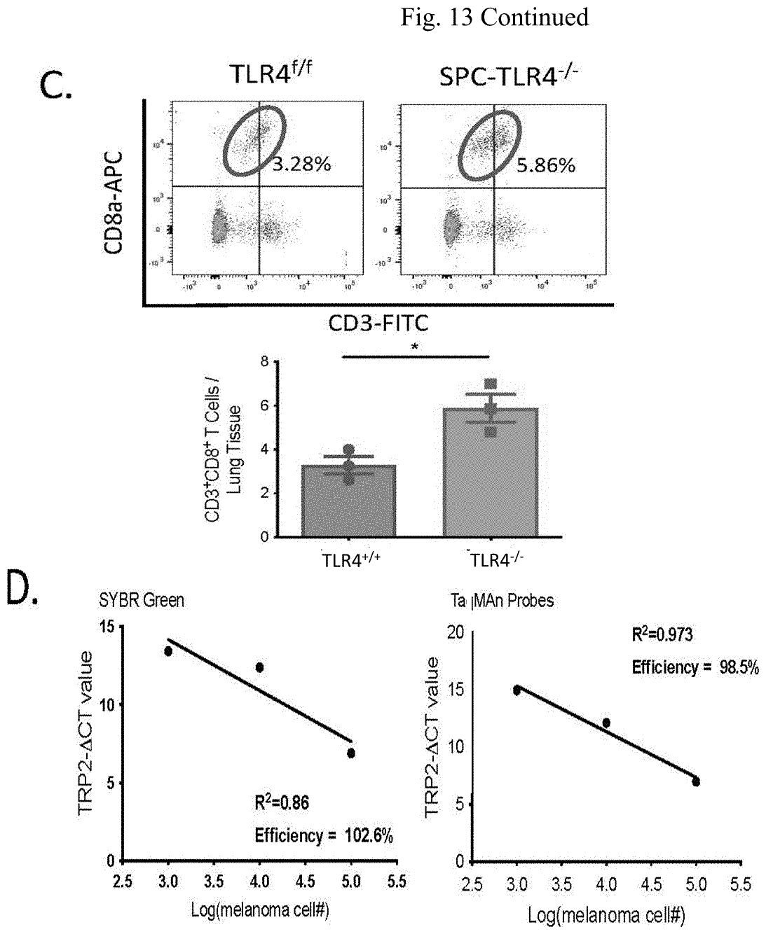

Despite prior reports indicating that the NLRP3 inflammasome can stimulate IL-1β production by melanoma cells, our previous studies have indicated that murine melanoma IL-1β levels are negligible relative to IL-1β production by myeloid cell populations in response to NLRP3 inflammasome activation (FIG. 12A) (19, 26, 27). Indeed, while we were able to detect soluble HSP70 as a secretion product of human melanoma cell lines, we were not able to detect the production of active IL-1β (FIGS. 12B and C). This is consistent with additional studies finding that tumor-intrinsic NLRP3 activation elicits the release of significantly higher levels of HSP70 relative to IL-1β, the inverse of what is observed in myeloid cell populations in response to various stimuli (FIGS. 12D and E). We have previously shown that the tumor-intrinsic NLRP3 inflammasome drives PMN-MDSC recruitment via an autocrine signaling pathway dependent on soluble HSP70 (19). However, we were also able to measure plasma levels of HSP70 in both the transgenic BRAF^{V600E}PTEN^{-/-} melanoma model as well as in melanoma patients, suggesting that HSP70 could also have systemic effects (19). To address whether primary BRAF^{V600E}PTEN^{-/-} melanomas can impact HSP70 levels in distant lung tissues, we performed both enzyme-linked immunosorbent assays (ELISAs) and Western blot analysis revealing elevated HSP70 levels in the lungs of BRAF^{V600E}PTEN^{-/-} melanoma-bearing mice relative to hosts harboring no primary tumor (FIGS. 2, A and B). Notably, we were unable to detect significant differences in HSP70 mRNA levels in lung tissues between tumor-bearing and non-tumor-bearing mice based on qrt-PCR analysis, indicating that detectable HSP70 protein was likely derived from the circulation. To determine whether HSP70 could play the role of a soluble mediator in the induction of CXCR2-dependent chemokines in the distant lung, we delivered recombinant HSP70 (rHSP70) into non-tumor-bearing mice by intra-peritoneal (i.p.) injection and measured CXCL5 expression by whole lung tissue Western blot analysis as well as PMN-MDSC accumulation in the lung by flow cytometry. Indeed, these studies showed the delivery of rHSP70 to induce CXCL5 expression and promote the accumulation of PMN-MDSCs in lung tissues (FIG. 2C, and FIG. 13A). Our prior studies have demonstrated that HSP70 signals through TLR4 to induce Wnt5a-mediated CXCR2 chemokine expression in tumor tissues and prior studies have implicated lung TLR4 signaling in the upregulation of CXCL5 by lung epithelial cells (19, 28). Based on this data, whole lung tissue Western blots were performed to identify an upregulation in Wnt5a expression by lung tissues in mice harboring a primary melanoma relative to non-tumor-bearing mice (FIG. 2D). Using a murine lung epithelial cell line (MLE12) along with a TLR4 inhibitor, we subsequently confirmed rHSP70 to induce Wnt5a expression in a TLR4-dependent manner (FIG. 2D). Based on these cumulative findings, we hypothesized that tumor-derived HSP70 signals through TLR4 to promote PMN-MDSC trafficking to these distant tissues. In order to address the importance of TLR4 signaling in lung epithelial cells in driving this process, we crossed a SPC-Cre-ER^{T2} transgenic mouse harboring a tamoxifen-inducible Cre recombinase (Cre-ER^{T2}) under the control of the human surfactant protein C (SPC) promoter with a mouse harboring a Tlr4 conditional knock-out allele (TLR4″) to generate SPC-Cre-ER^{T2}/TLR4^{fl/fl} offspring (SPC-TLR4^{-/-}) (FIG. 13B) (29). Employing the SPC-TLR4^{-/-} transgenic mouse model and flow cytometry analysis, we found lung epithelial cell-specific TLR4 deletion to suppress PMN-MDSC accumulation in the lung while increasing CD8⁺ T cell infiltration into these tissues (FIG. 2E, and FIG. 13C). This observation correlated with a reduction in the expression of both Wnt5a and Cxcl5 by lung epithelial cells upon TLR4 ablation in SPC-TLR4^{-/-} mice (FIG. 2F, and FIG. 13B). To confirm that these findings in the SPC-TLR4^{-/-} transgenic mice were, in fact, dependent upon systemic HSP70, we treated these mice with rHSP70 delivered by i.p. injection and found both Wnt5a and Cxcl5 to be upregulated only in lung epithelial cells expressing TLR4 (FIG. 2G). These cumulative data indicate that systemic HSP70 triggers a TLR4-Wnt5a signaling axis in lung epithelial tissues to drive PMN-MDSC accumulation in the lungs.

A TLR4-Wnt5a Signaling Axis in Lung Epithelial Tissues Promotes Pulmonary Metastatic Progression

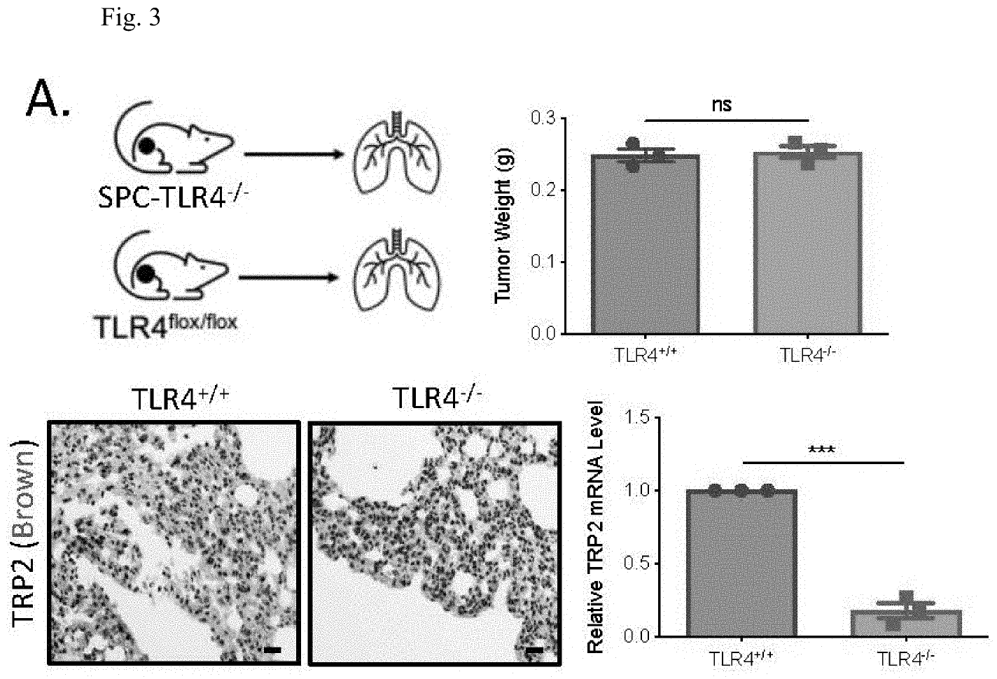

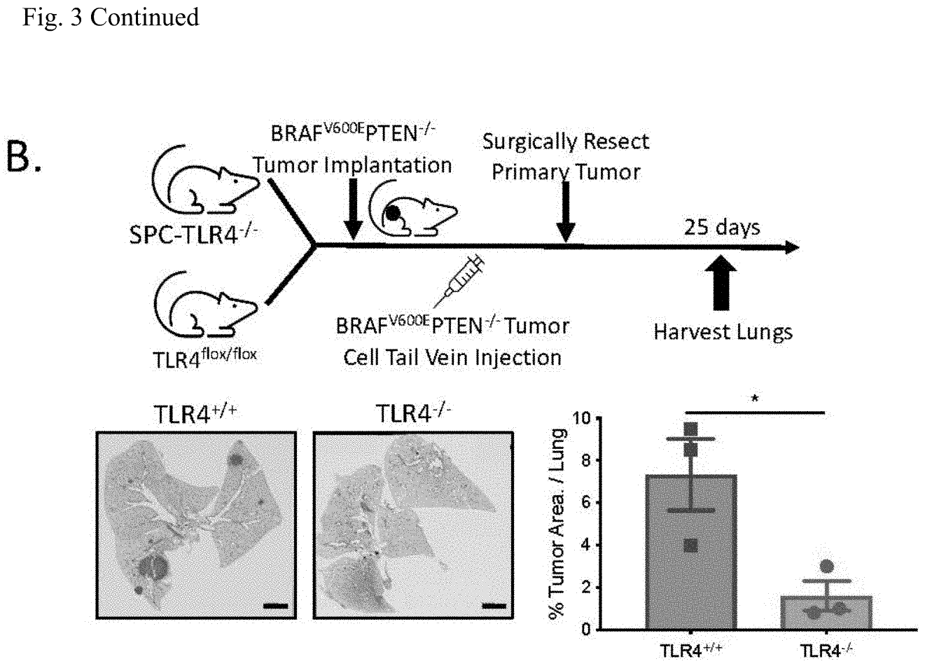

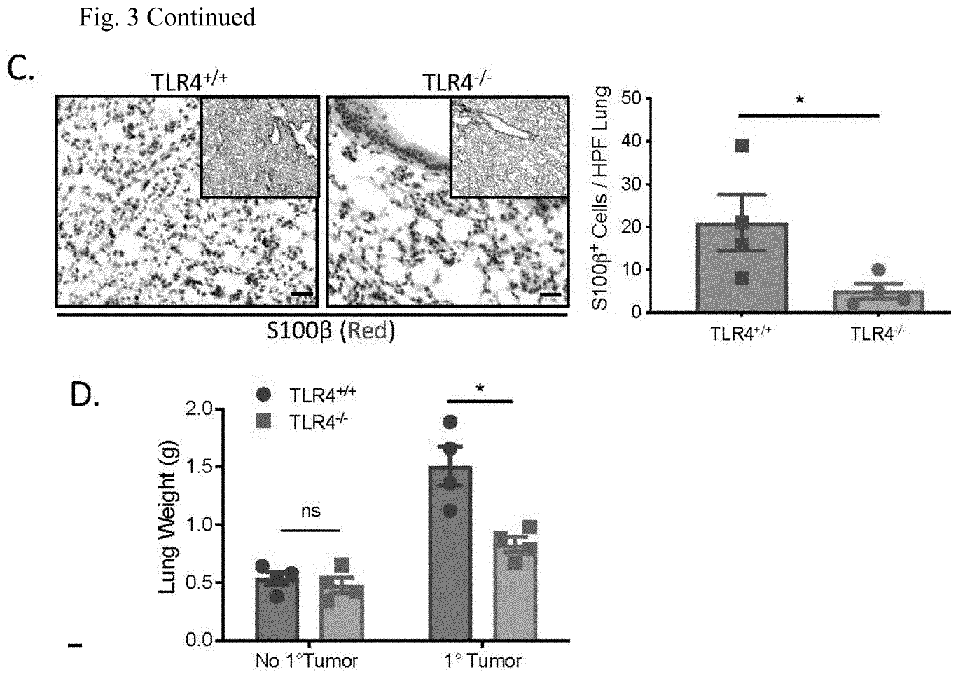

Prior studies have shown gain-of-function polymorphisms of TLR4 to associate with increased metastatic progression and inferior survival in cancer patients (13, 30, 31). In addition, recent work has further described a role for PMN-MDSCs in establishing a microenvironment in tissues that is more conducive to metastatic progression (1). Based on these findings, we investigated the role of lung epithelial TLR4 signaling in metastatic progression to the lung. We there implanted the BRAF^{V600E}PTEN^{-/-} melanoma cell line by subcutaneous injection into syngeneic SPC-TLR4^{-/-} and TLR4^{fl/fl} control hosts and resected the primary melanoma tissues before harvesting the lungs three weeks later. While the primary tumor tissues exhibited no difference in weight between SPC-TLR4^{-/-} and TLR4^{fl/fl} control mice, lung tissues derived from SPC-TLR4^{-/-} mice were associated with diminished expression levels of the tyrosine-related peptide-2 (TRP2) melanoma antigen based on both TRP2 immunohistochemistry (IHC) and a previously developed TRP2-targeted qrt-PCR assay for quantifying melanoma metastases (FIG. 3A, and FIG. 13D) (32). The syngeneic BRAF^{V600E}PTEN^{-/-} melanoma model does not readily metastasize to distant tissues spontaneously. Therefore, to further study the metastasis of this model to the lung, we again implanted the BRAF^{V600E}PTEN^{-/-} melanoma cell line by subcutaneous injection in both SPC-TLR4^{-/-} and TLR4^{fl/fl} control mice to induce PMN-MDSC accumulation at distant tissue sites. These mice were then administered the BRAF^{V600E}PTEN^{-/-} melanoma cell line by tail vein injection and lung tissues were harvested 25 days later for hematoxylin and eosin (H&E) microscopy, IHC analysis of the S100β melanoma antigen, as well as lung weight measurements (FIG. 3, B to D). Altogether, this work confirmed that the establishment of lung metastases is dependent upon TLR4 signaling in lung epithelial cells as well as the presence of a primary tumor.

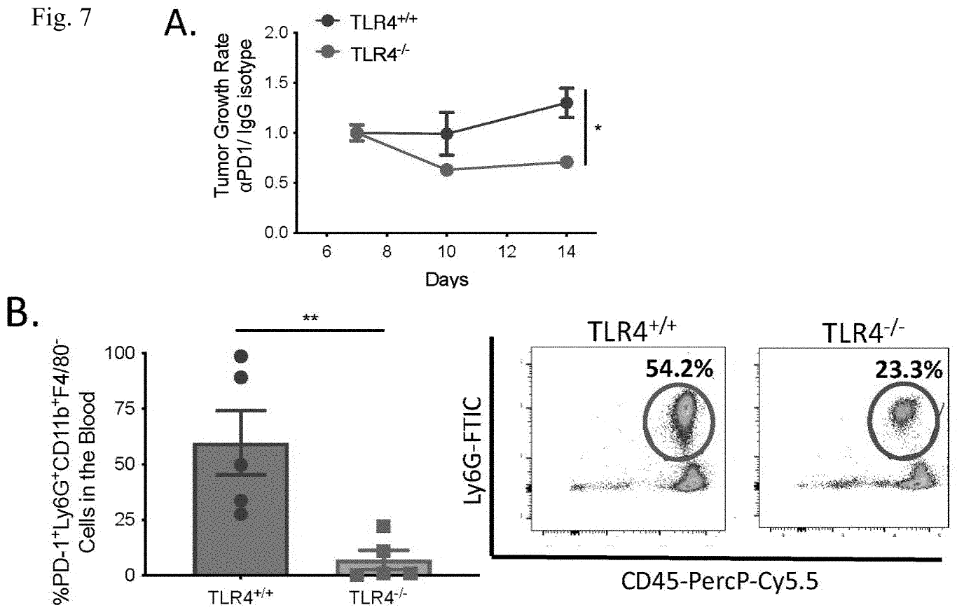

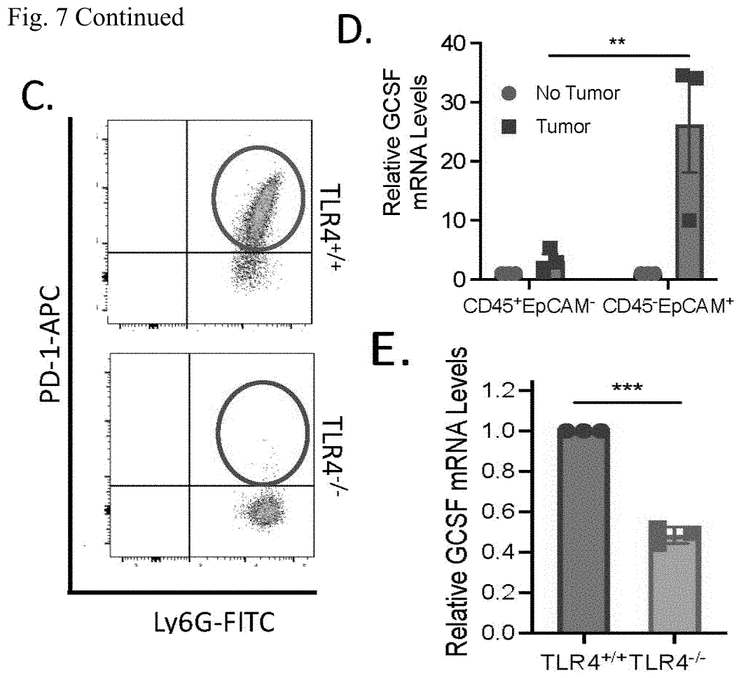

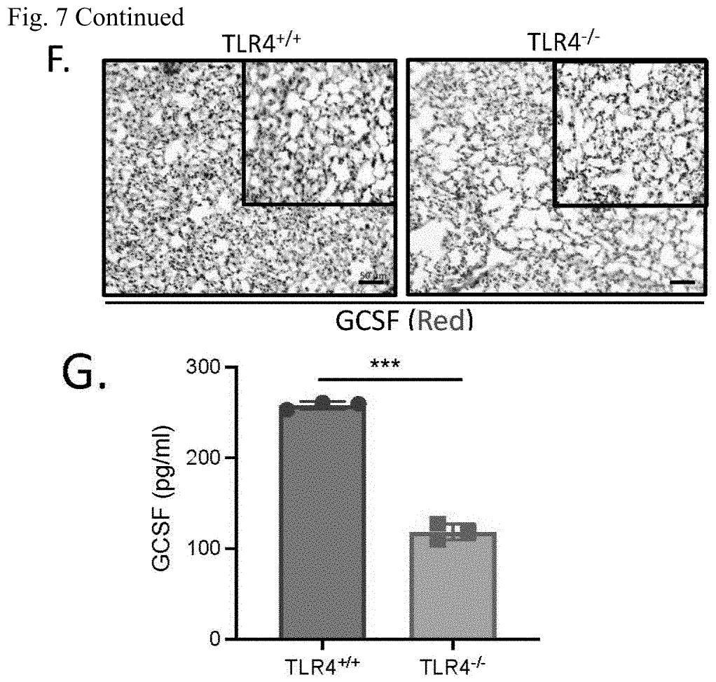

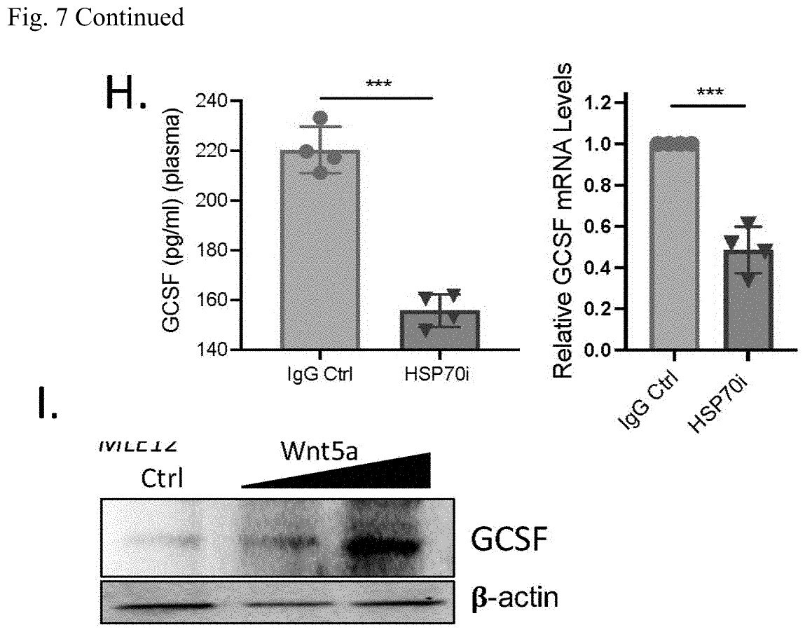

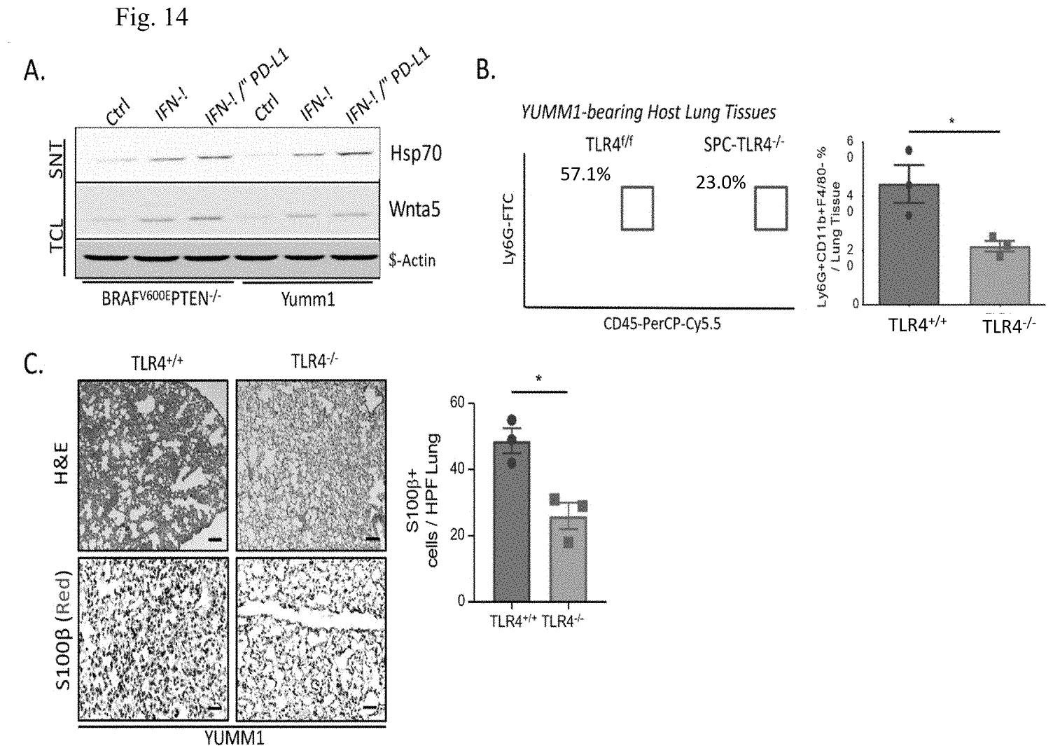

The delivery of tumor cells via tail vein injection circumvents critical steps in the process of metastasis, representing a more artificial system for studying tumor progression. We therefore turned to tumor models capable of spontaneously metastasizing to the lung, including the BRAF^{V600E}CDKN2A^{-/-}PTEN^{-/-} (YUMM1) melanoma model as well as a tumor model of a different histology, the E0771 breast adenocarcinoma model (33, 34). Our findings in each of these additional models further confirmed our prior data, demonstrating a critical role for lung epithelial TLR4 signaling in driving PMN-MDSC accumulation and metastatic progression to the lung and verify that this phenomenon is not restricted to BRAF^{V600E}PTEN^{-/-} melanomas (FIGS. 14 and 7).

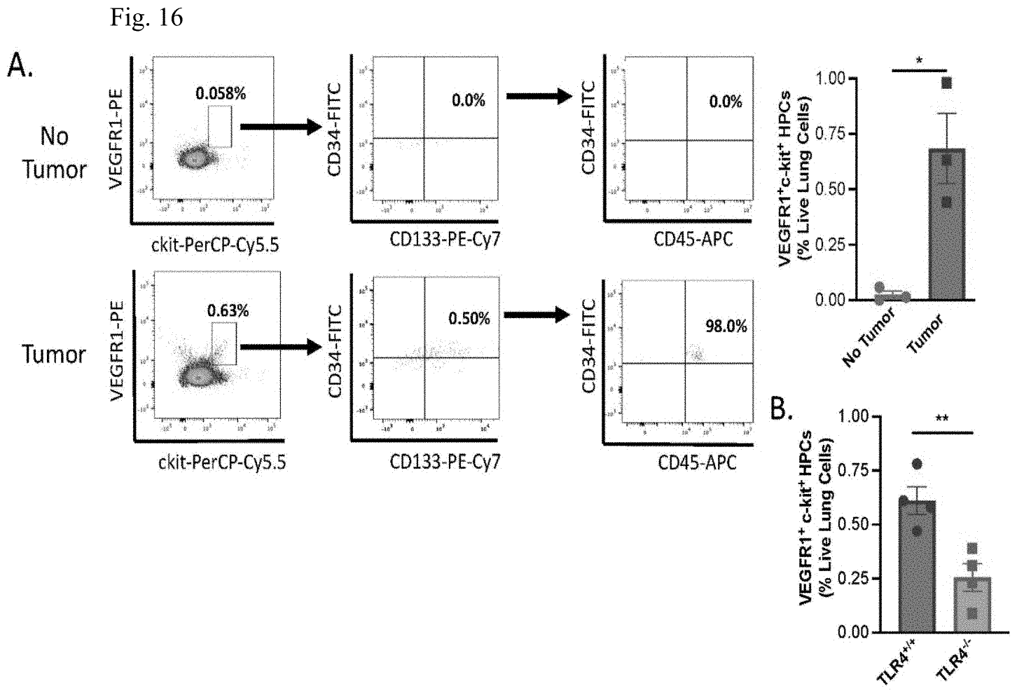

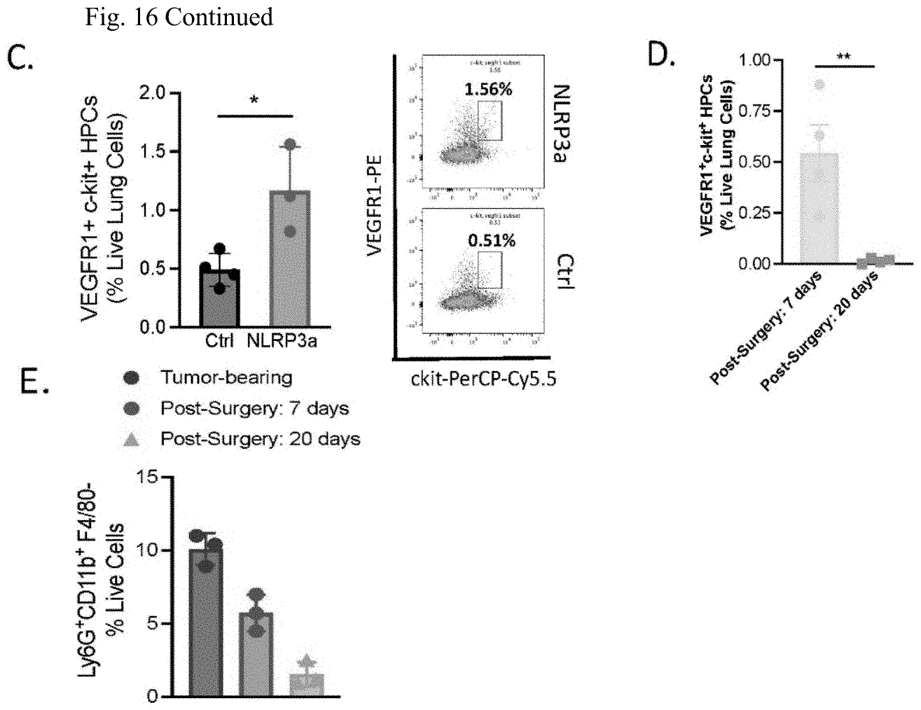

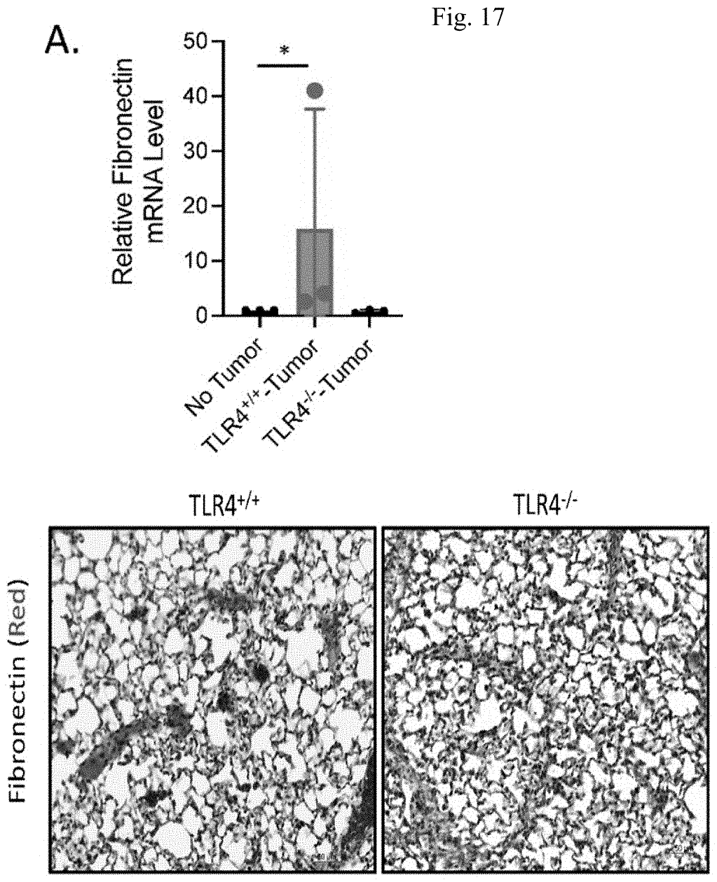

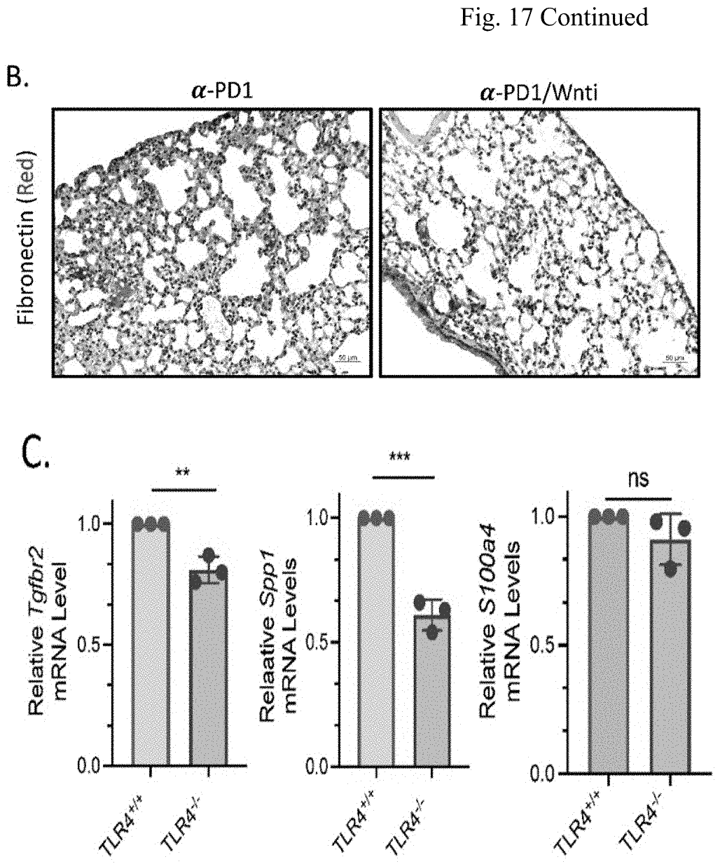

Prior studies have shown that the arrival of hematopoietic cell populations (HPC) expressing vascular endothelial growth factor receptor-1 (VEGFR1) in the lung as well as increased fibronectin expression by local fibroblasts contribute to the formation of a pre-metastatic niche that subsequently supports metastatic progression (35). Using multi-parameter flow cytometry, we indeed observed an increase in a c-kit⁺CD133⁺CD34⁺VEGFR1⁺ HPC (VEGFR1⁺ HPC) population in mice harboring BRAF^{V600E}PTEN^{-/-} melanomas relative to non-tumor-bearing controls. The accumulation of this VEGFR1⁺ HPC population was also found to be dependent upon lung epithelial TLR4 expression and to be enhanced by tumor-intrinsic NLRP3 inflammasome activation (FIG. 16). Additional qrt-PCR and IHC studies further demonstrated enhanced fibronectin expression levels in the lungs of mice bearing BRAF^{V600E}PTEN^{-/-} melanomas and for this upregulation to be reversed in SPC-TLR4^{-/-} mice as well as mice treated with an inhibitor to the Wnt signaling regulator, PORCN (ETC-159) (FIG. 17). Altogether, these results indicate that the tumor NLRP3-lung epithelial TLR4 axis represents an early step in pre-metastatic niche development.

Anti-PD-1 Immunotherapy Drives PMN-MDSC Accumulation in the Lung Via the Tumor-Intrinsic NLRP3-HSP70 Axis

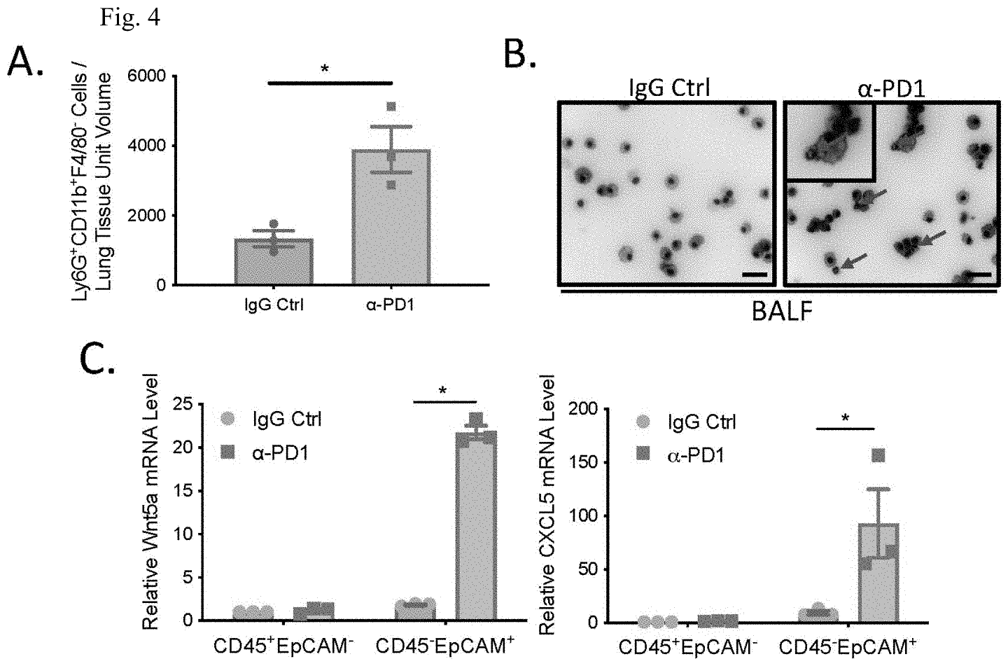

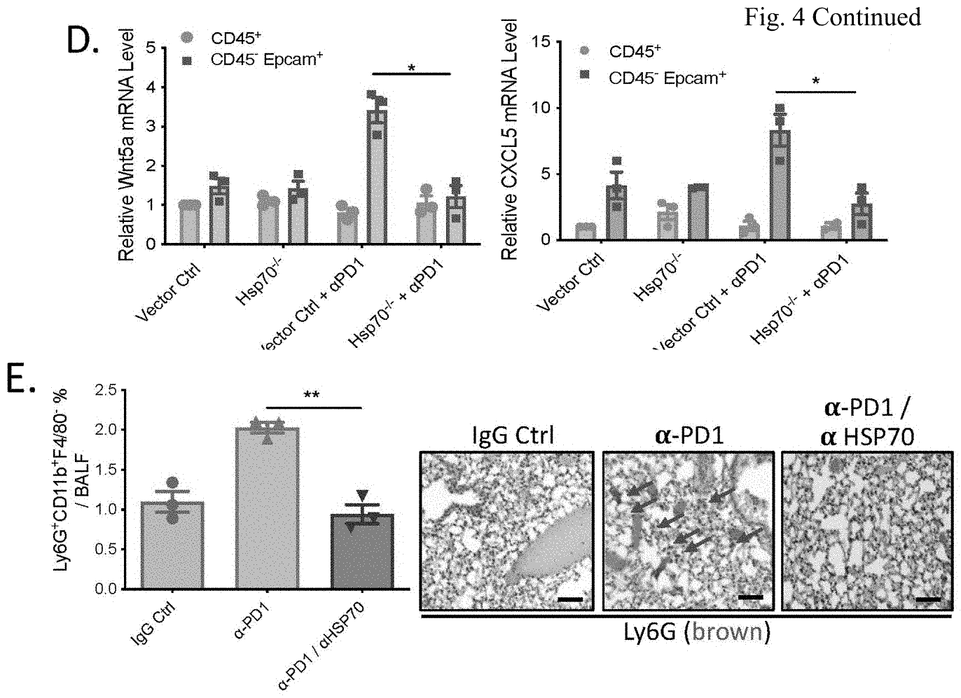

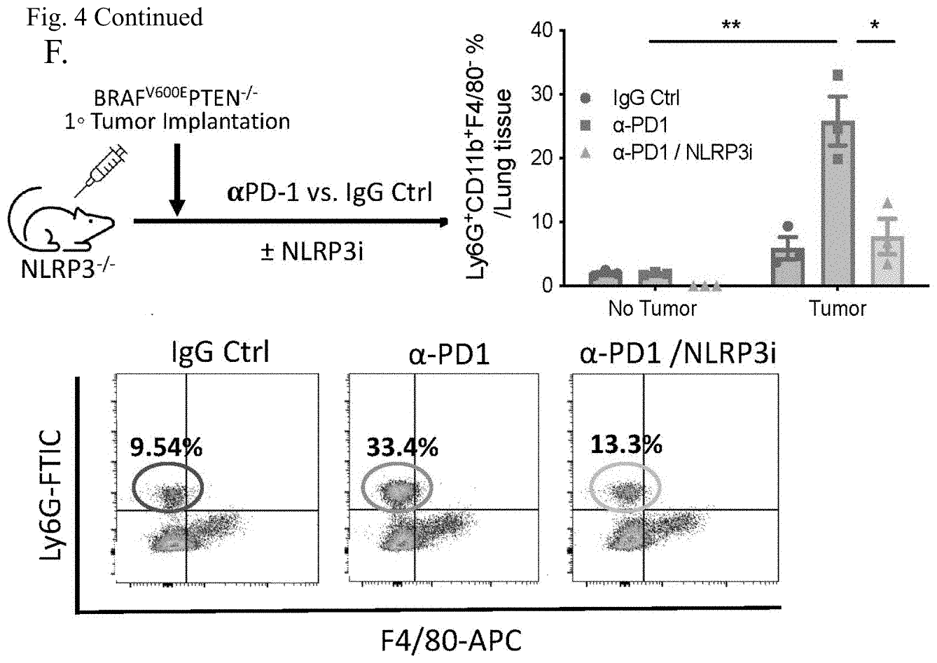

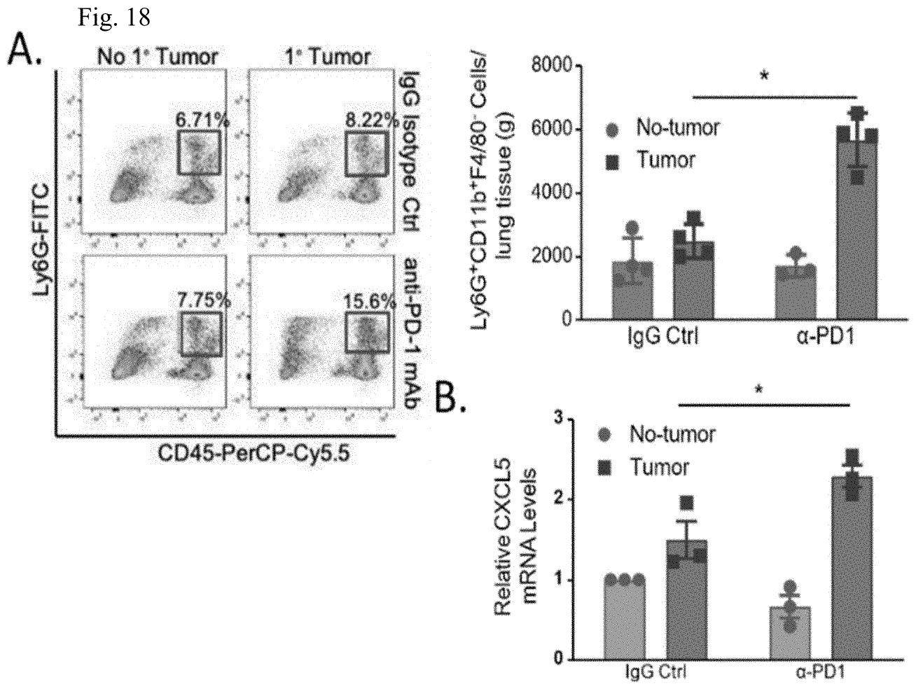

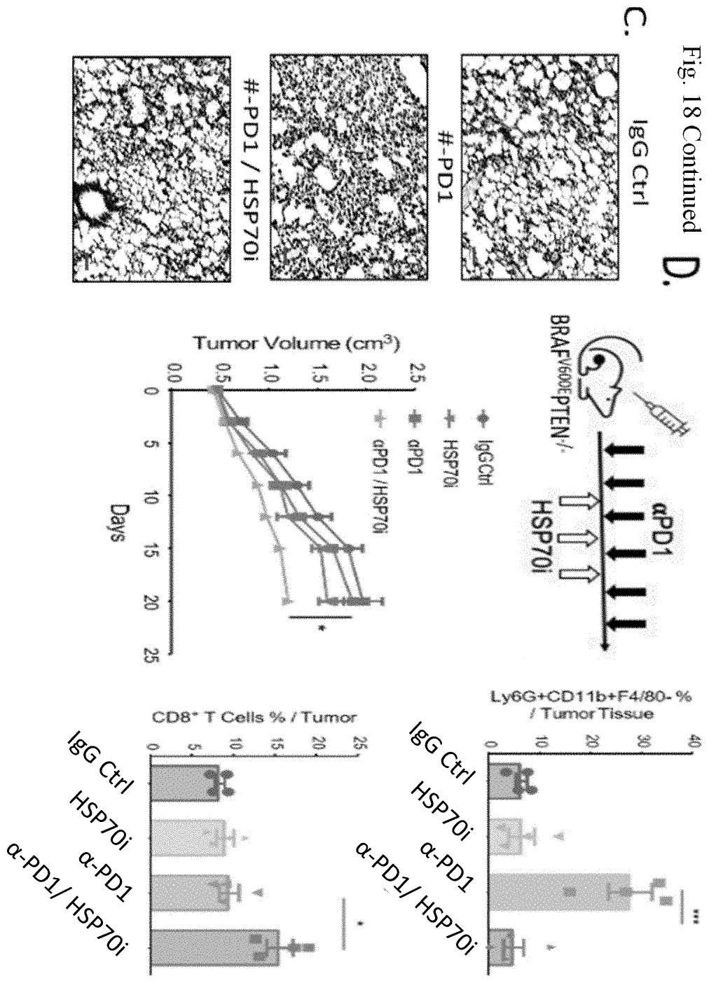

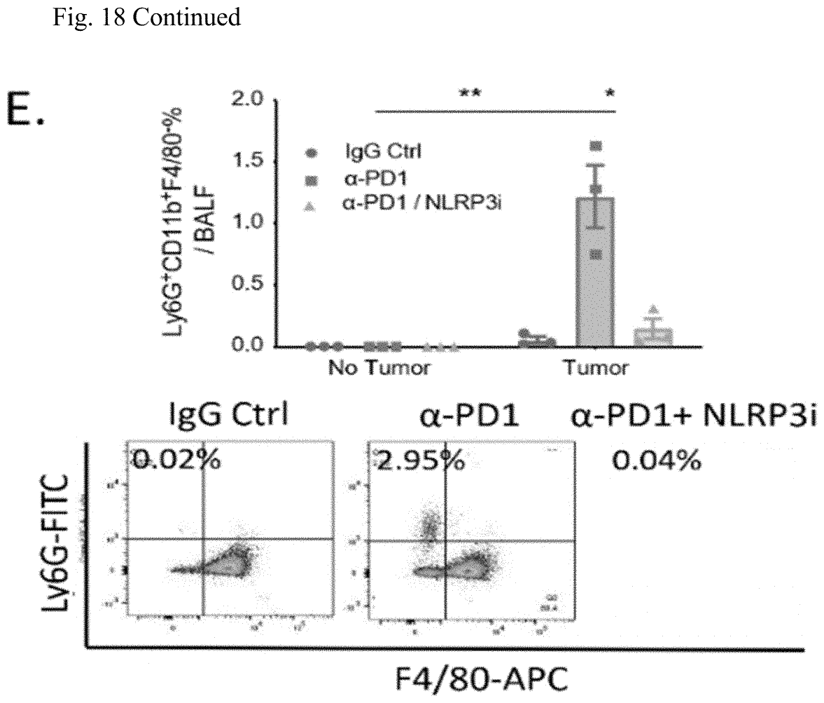

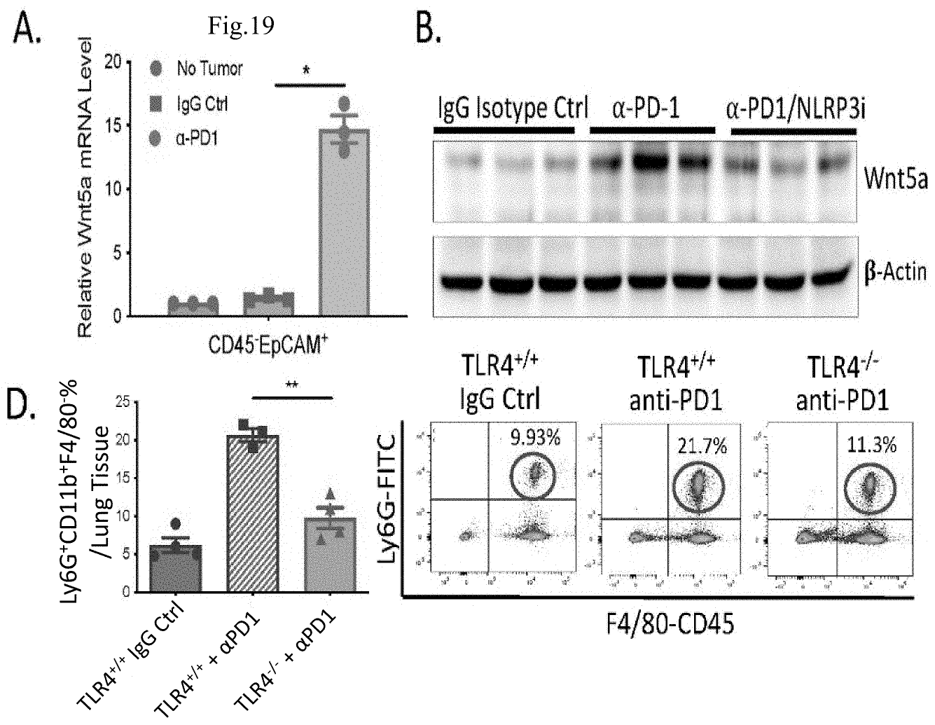

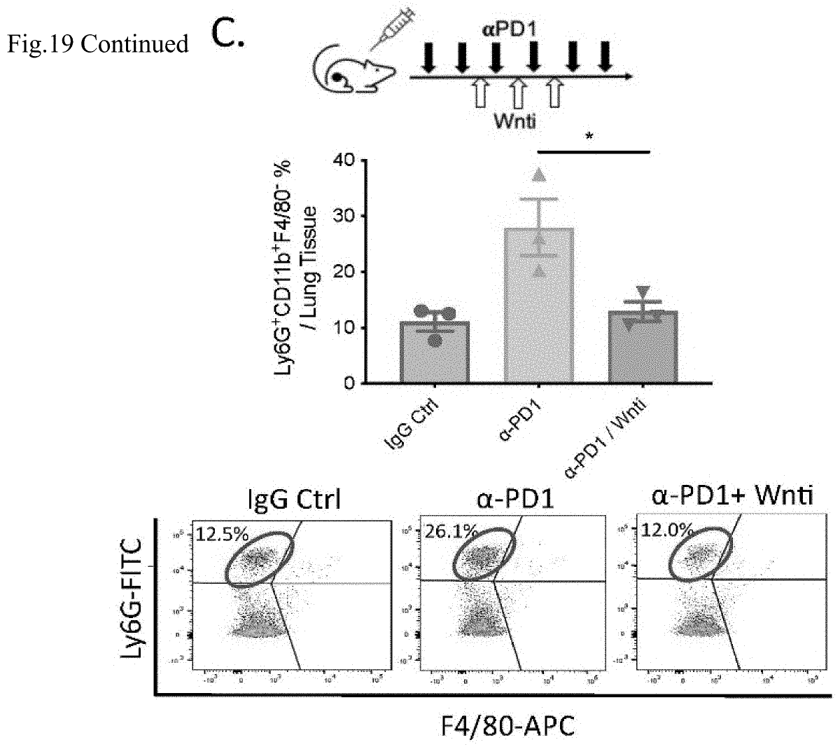

We previously demonstrated that anti-PD-1 immunotherapy promotes the recruitment of PMN-MDSCs into the primary tumor bed by inducing the activation of a tumor-intrinsic NLRP3 inflammasome-HSP70 signaling axis (FIG. 10B) (19). Consistent with our previous data, we have further determined that anti-PD-1 immunotherapy drives PMN-MDSC accumulation in the lung based on tissue flow cytometry as well as bronchoalveolar fluid (BALF) cytology studies, an effect that is only observed in tumor-bearing mice (FIGS. 4A and B, and FIG. S18A). These observations are also consistent with studies showing that anti-PD-1 immunotherapy induces the expression of both Wnt5a and Cxcl5 by CD45⁻EpCAM⁺ lung epithelial cells only in tumor-bearing mice (FIG. 4C, and FIGS. 10A and 18B). To determine whether tumor-derived HSP70 is responsible for these observed alterations in response to PD-1 blockade, we knocked-out HSP70 in the BRAF^{V600E}PTEN^{-/-} melanoma model using CRISPR/Cas9 (BRAF^{V600E}PTEN^{-/-}-HSP70^{-/-}) (19). While we observed an increase in both Wnt5a and Cxcl5 expression by CD45⁻EpCAM⁺ lung epithelial cells in response to anti-PD-1 immunotherapy in BRAF^{V600E}PTEN^{-/-} melanoma-bearing mice, this effect was eliminated in mice harboring BRAF^{V600E}PTEN^{-/-}-HSP70^{-/-} melanomas (FIG. 4D). In line with this data, additional studies further showed an antagonistic antibody to HSP70 to suppress PMN-MDSC numbers in the lung when given in combination with anti-PD-1 immunotherapy (FIG. 4E, and FIG. 18C). Notably, this response to a HSP70 inhibitor was observed to also correlate with a more robust anti-tumor immune response when administered in combination with anti-PD-1 immunotherapy (FIG. 18D). To further determine that anti-PD-1 immunotherapy also required the tumor NLRP3 inflammasome to drive PMN-MDSC accumulation in the lung, we treated BRAF^{V600E}PTEN^{-/-} melanoma-bearing NLRP3^{-/-} mice with anti-PD-1 antibody alone or in combination with the NLRP3 inhibitor, MCC950. While anti-PD-1 therapy strongly induced the accumulation of PMN-MDSCs into lung tissues in tumor-bearing NLRP3^{-/-} mice based on flow cytometry analysis of both lung tissue and BALF, this effect was largely eliminated by treatment with MCC950 (FIG. 4F, and FIG. 1E). Consistent with a role for HSP70-induced Wnt5a in the accumulation of PMN-MDSCs at distant sites, we noted MCC950 to suppress the upregulation of Wnt5a expression in lung tissues and for Wnt ligand inhibition to phenocopy the observed effect of NLRP3 inhibition on PMN-MDSC recruitment into lung tissues in response to anti-PD-1 immunotherapy (FIG. 19, A to C). In line with our prior studies, the genetic deletion of TLR4 specifically in lung epithelial tissues also eliminates PMN-MDSC accumulation in response to anti-PD-1 immunotherapy (FIG. 19D). Overall, these findings imply that NLRP3-dependent release of HSP70 from tumors undergoing anti-PD-1 immunotherapy promotes the accumulation of PMN-MDSCs in distant lung tissues via the induction of a local TLR4-Wnt5a signaling pathway.

The Tumor-Intrinsic NLRP3-HSP70 Axis can Facilitate Disease Hyperprogression in Response to Anti-PD-1 Immunotherapy

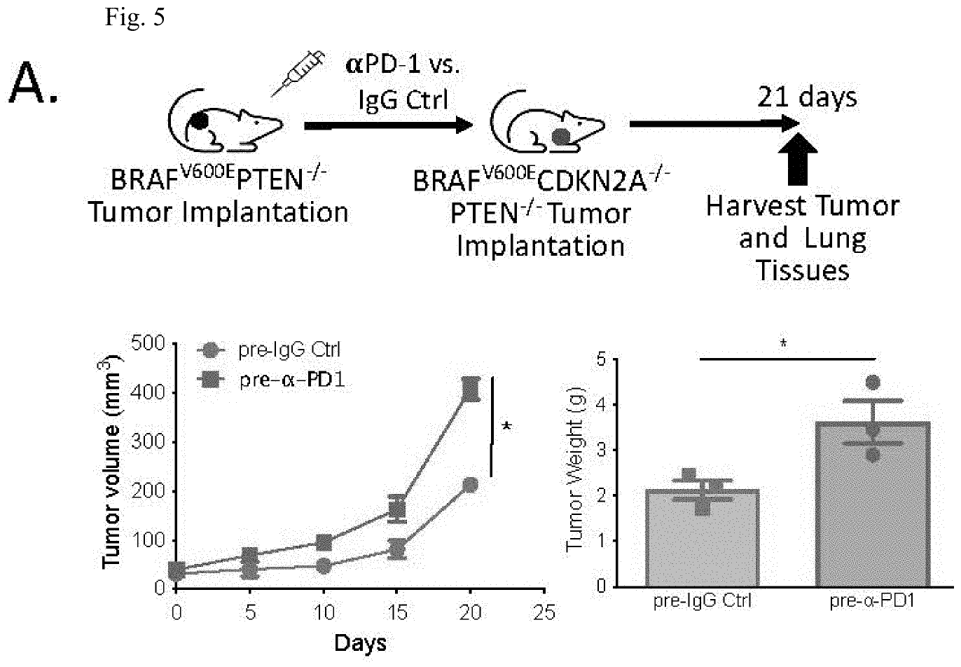

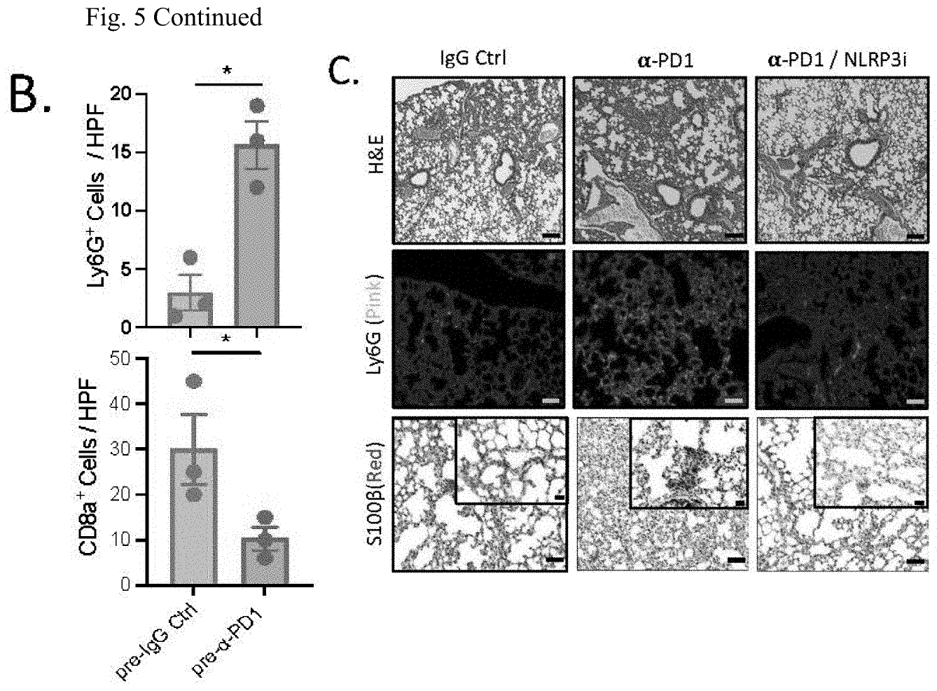

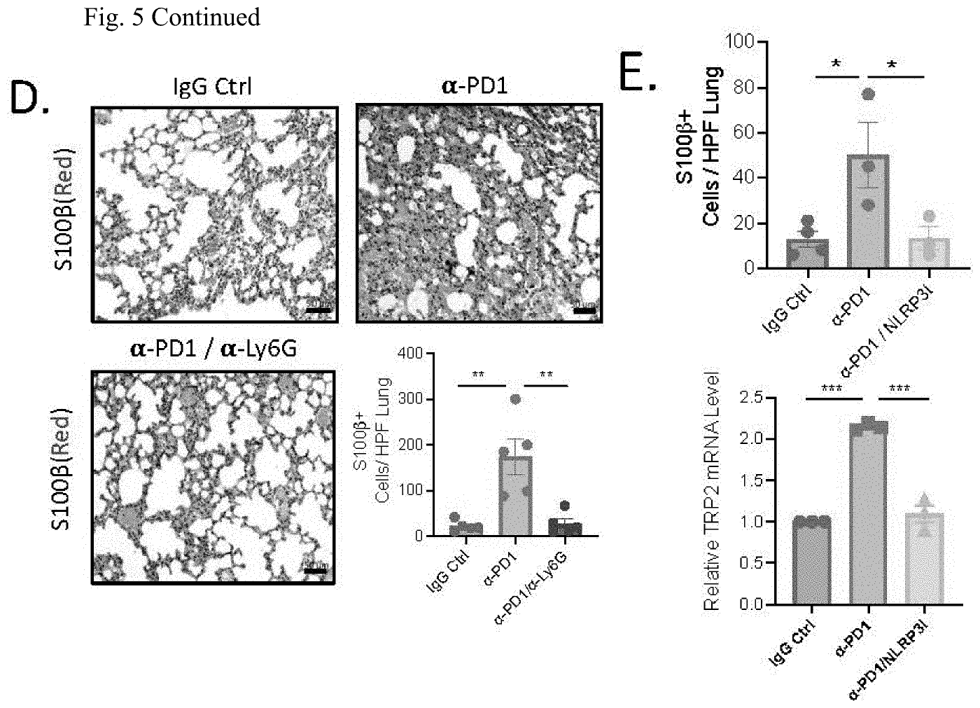

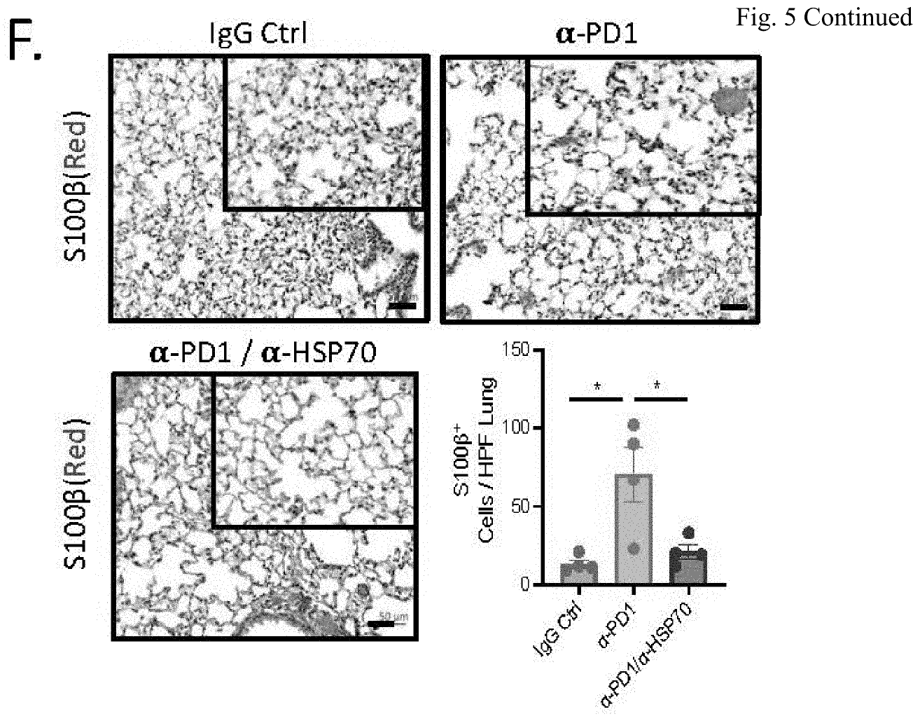

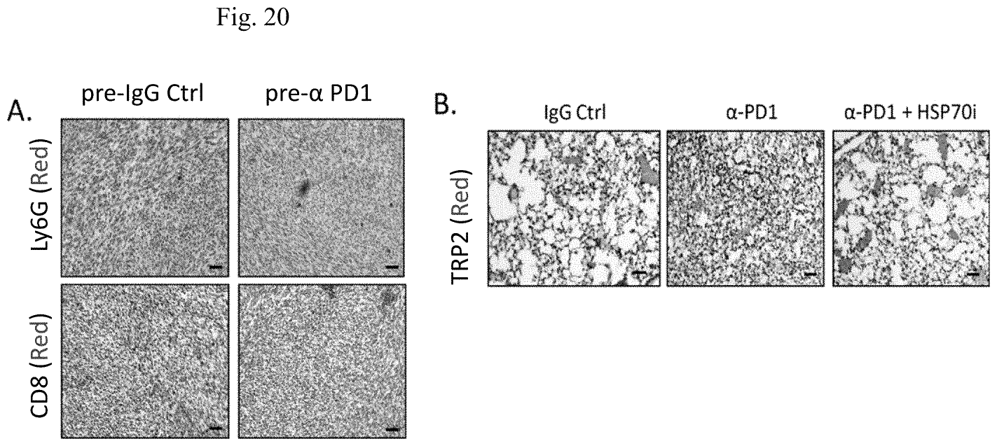

Prior studies have described disease hyper-progression (HPD) occurring in response to anti-PD-1 immunotherapy, a phenomenon estimated by some authors to develop in ˜10% of all solid tumors (5). In HPD, tumor burden can dramatically increase in response to exposure to anti-PD-1 immunotherapy. Given our prior data describing a role for the tumor-intrinsic NLRP3-HSP70 signaling axis as an important driver of PMN-MDSC accumulation in distant lung tissues in response to anti-PD-1 immunotherapy, we hypothesized that this same tumor-intrinsic signaling pathway can establish an immunologic environment capable of facilitating HPD. To address this question, we first treated transplanted syngeneic BRAF^{V600E}PTEN^{-/-} melanomas with anti-PD-1 therapy. This approach suppressed primary BRAF^{V600E}PTEN^{-/-} melanoma growth as previously observed (36, 37). However, if we subsequently transplanted these same mice harboring anti-PD-1-treated BRAF^{V600E}PTEN^{-/-} melanomas with the YUMM1 melanoma model, we observed evidence of more rapid disease progression relative to those mice previously treated with IgG control antibody (FIG. 5A). Further IHC studies revealed that those progressive primary YUMM1 melanomas following pre-treatment with anti-PD-1 were also found to harbor increased numbers of PMN-MDSCs and decreased levels of infiltrating CD8⁺ T cells (FIG. 5B, FIG. 20A). Based on these results, we examined the impact of anti-PD-1 immunotherapy on the course of the autochthonous BRAF^{V600E}PTEN^{-/-} melanoma model and sought to determine how PMN-MDSCs influenced the behavior of these tumors. While a modest therapeutic effect is generally observed in response to anti-PD-1 immunotherapy in this model, we have uniformly observed disease escape and progression thereafter (36, 37). Utilizing the S10013 melanoma antigen as a marker, we noted a significant increase in pulmonary metastasis following anti-PD-1 immunotherapy, a finding that correlated with increased numbers PMN-MDSCs in the lungs (FIG. 5C). Consistent with our previous data which implicated PMN-MDSCs as playing a significant role in promoting the development of a distant pre-metastatic niche, the ablation of circulating PMN-MDSCs nearly eliminated metastatic progression to the lung (FIG. 5D). Further in-line with these results, additional studies conducted in the transgenic BRAF^{V600E}PTEN^{-/-} melanoma model demonstrated the NLRP3 inhibitor, MCC950, to suppress PMN-MDSC populations in the lung while also inhibiting metastatic progression based on both S100β IHC and TRP2 qrt-PCR analysis (FIGS. 5C and E). Suppression in distant lung metastases based on both S100β and TRP2 IHC was also observed with an antagonistic anti-HSP70 antibody, consistent with an important role for the NLRP3-HSP70 signaling axis in driving distant metastatic progression (FIG. 5F, FIG. 20B).

Together, these data suggest that the tumor NLRP3-HSP70 axis can promote metastatic progression in response to anti-PD-1 immunotherapy under select conditions. Our prior work has demonstrated that this signaling axis contributes to an adaptive resistance mechanism to checkpoint inhibitor immunotherapy (19). Therefore, based on these data, we propose that adaptive mechanisms of anti-PD-1 resistance can evolve into HPD when this mechanism overwhelms the cytolytic T cell response. Whether specific germline or somatic genetic alterations impacting the NLRP3 signaling pathway are necessary to facilitate this response to anti-PD-1 therapy is unclear.

Genetic Amplification of NLRP3 Promotes Disease Hyperprogression in Response to Anti-PD-Immunotherapy

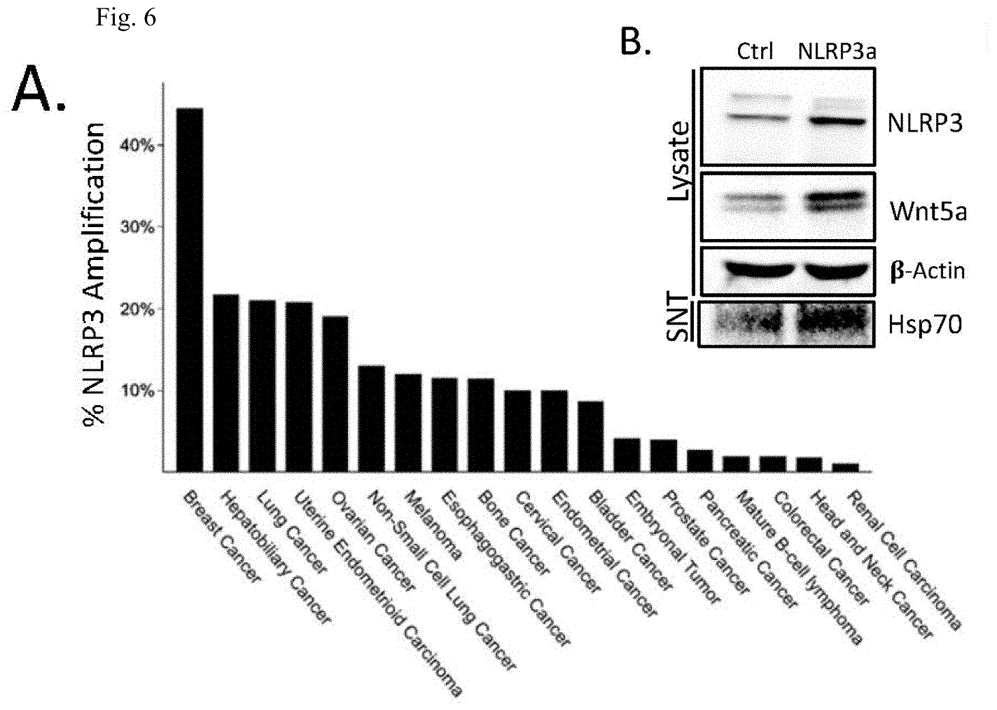

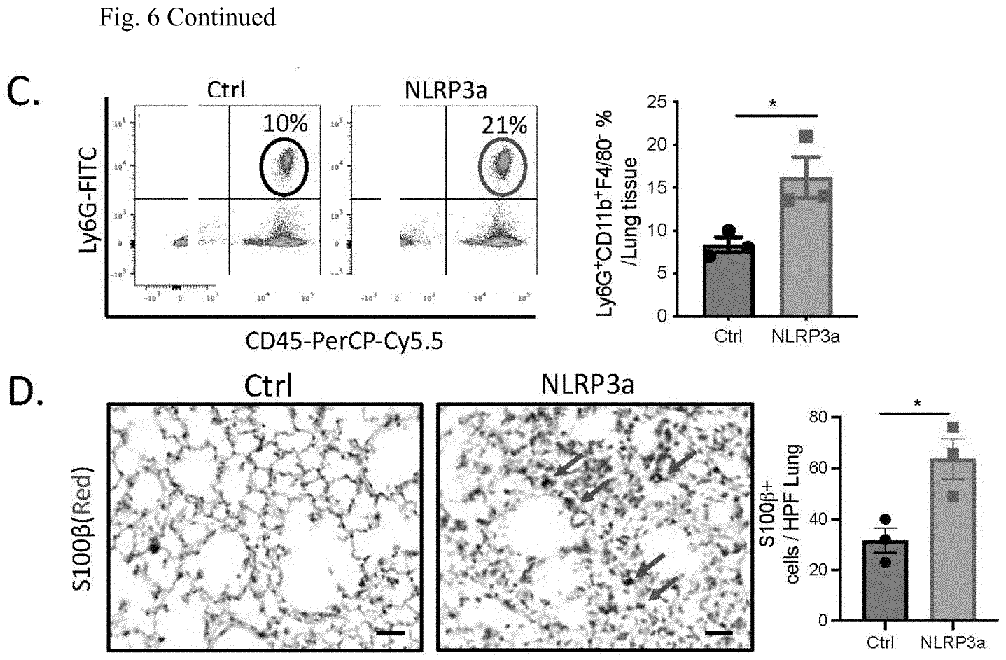

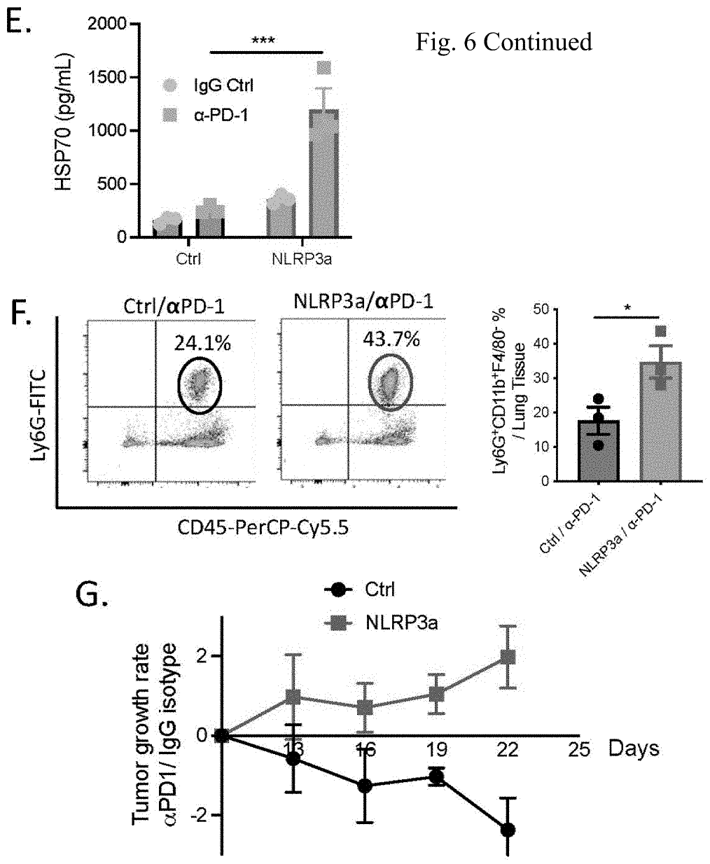

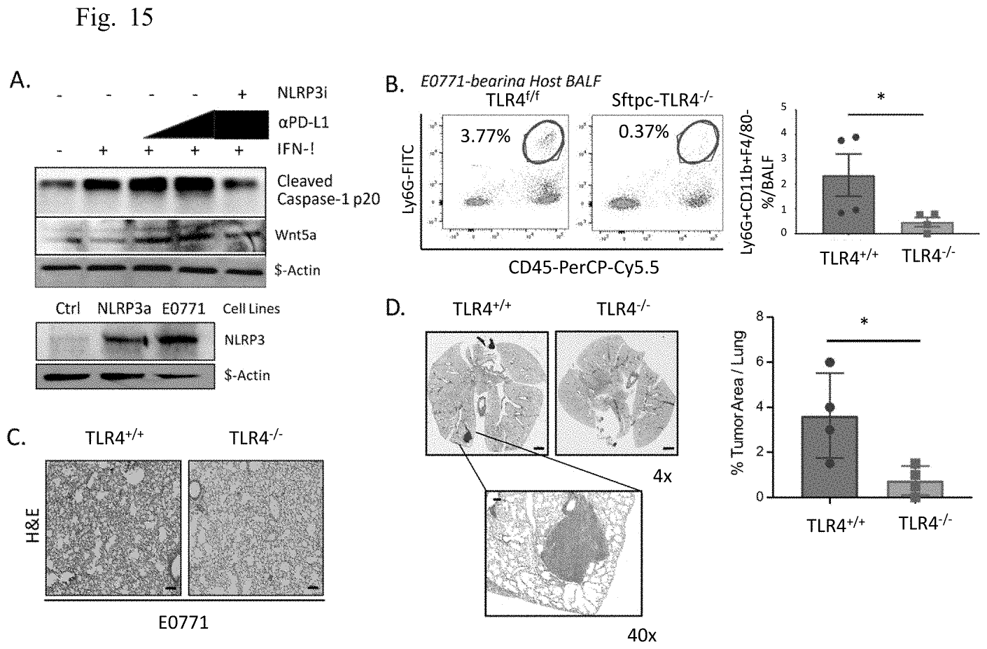

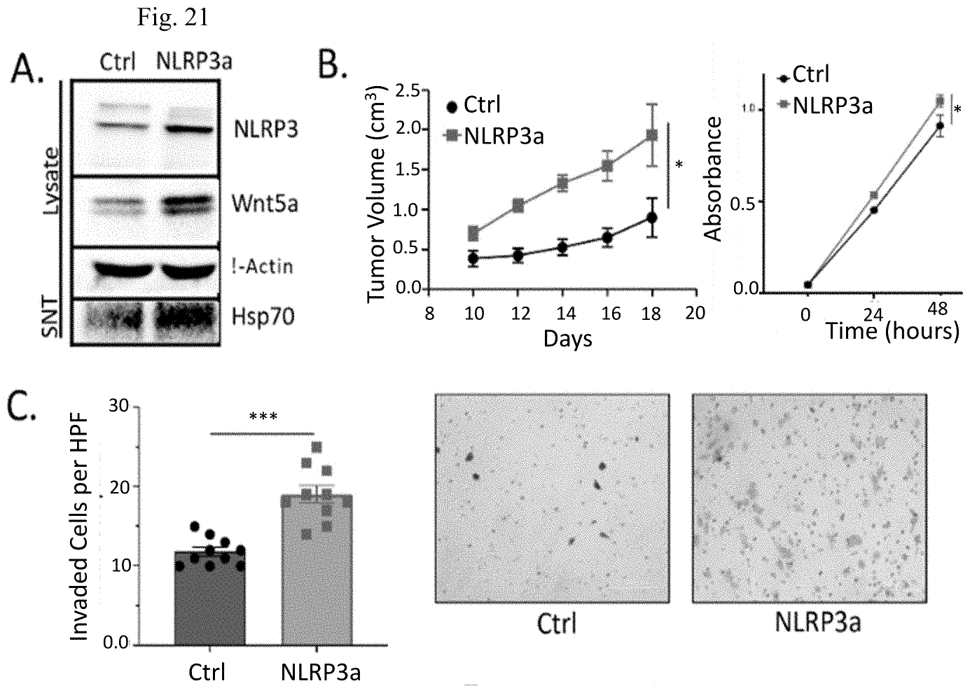

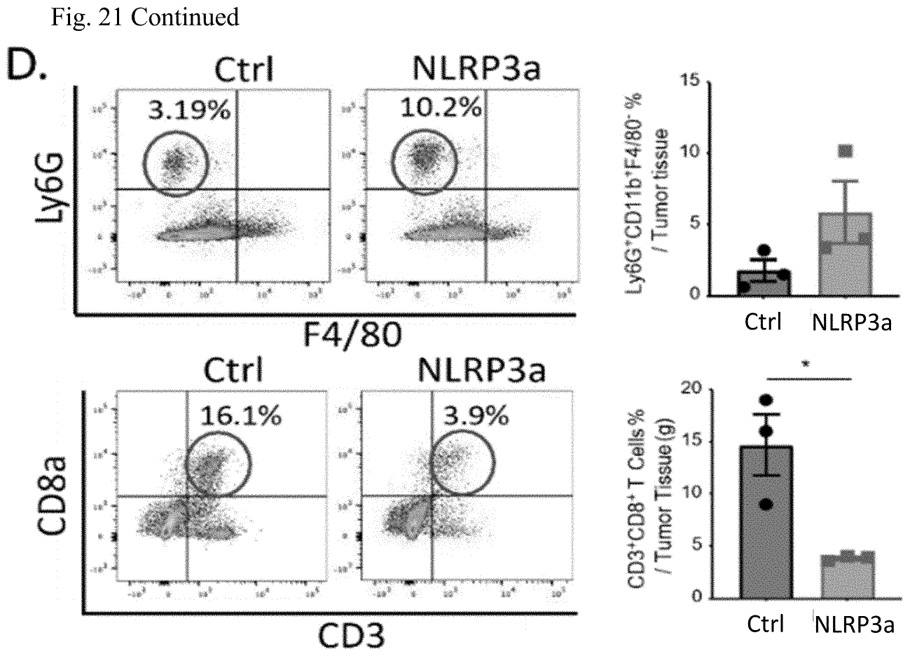

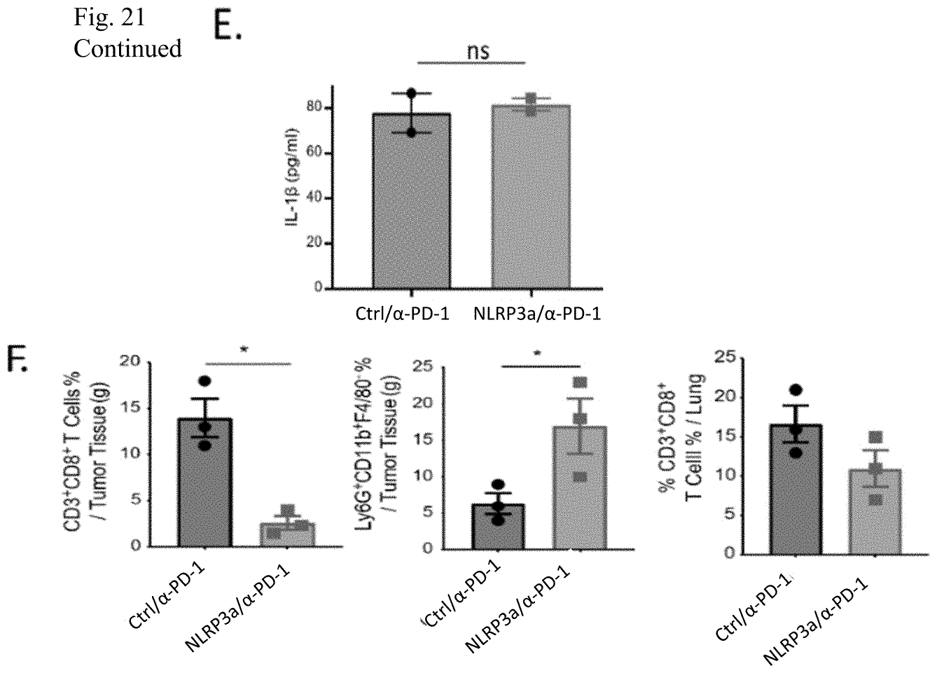

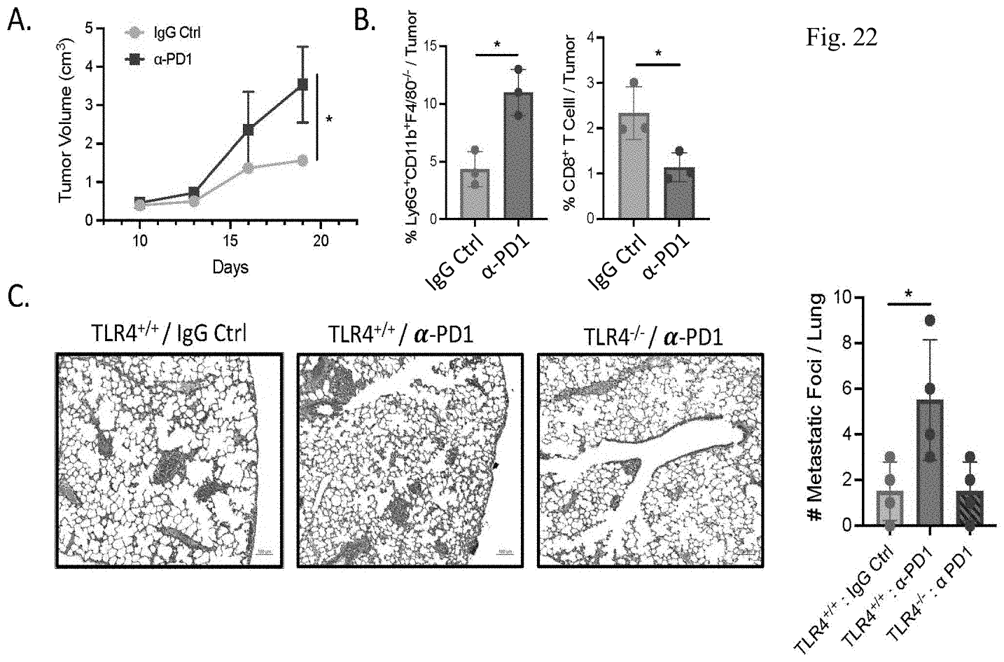

Many tumors exhibit elevated NLRP3 expression levels relative to their normal tissue counterparts (20). Indeed, amplification of NLRP3 has been identified in several solid tumor types (FIG. 6A) (38). We therefore hypothesized that genetic alterations that impact the activity of the NLRP3 inflammasome signaling pathway may influence whether a tumor exhibits resistance or possibly HPD in response to anti-PD-1 immunotherapy. To emulate these conditions, we utilized CRISPR activation (CRISPRa) technology to engineer a BRAF^{V600E}PTEN^{-/-} melanoma cell line to exhibit transcriptional activation of the Nlrp3 gene (BRAF^{V600E}PTEN^{-/-}-NLRP3a) (FIG. 13A). Consistent with prior studies, Nlrp3 amplification was found to promote tumor growth in vivo as well as tumor cell proliferation and invasion in vitro, suggesting that the NLRP3 inflammasome can contribute to tumor-intrinsic properties of growth and progression (FIGS. 21B and C). Additional in vivo studies also demonstrated BRAF^{V600E}PTEN^{-/-}-NLRP3a melanomas to exhibit increased PMN-MDSC accumulation in distant lung and local tumor tissues (FIG. 6B and FIG. 21D). These findings were also found to be associated with enhanced levels of lung metastases based on S100β IHC (FIG. 6C). Consistent with our previous studies implicating a role for the TLR4-Wnt5a axis in establishing a pre-metastatic niche in the lung, the impact of tumor NLRP3 amplification on lung PMN-MDSC accumulation as well as Wnt5a expression was verified to be dependent upon lung epithelial TLR4 signaling (FIG. 6D). Additional work further demonstrated NLRP3 amplification to enhance tumor-dependent HSP70 secretion, PMN-MDSC accumulation, and disease progression in response to PD-1 blockade (FIG. 6, E to G, and FIGS. 21, E and F). Interestingly, we found a similar response to anti-PD-1 immunotherapy in the E0771 breast cancer model which we previously determined to express elevated NLRP3 levels de novo relative to the NLRP3-amplified BRAF^{V600E}PTEN^{-/-} melanoma model (FIGS. 15A and 22). Altogether, these data suggest that tumors harboring genetic alterations associated with enhanced expression and/or activation of the NLRP3 inflammasome are more likely to exhibit resistance and even HPD in response to anti-PD-1 immunotherapy.

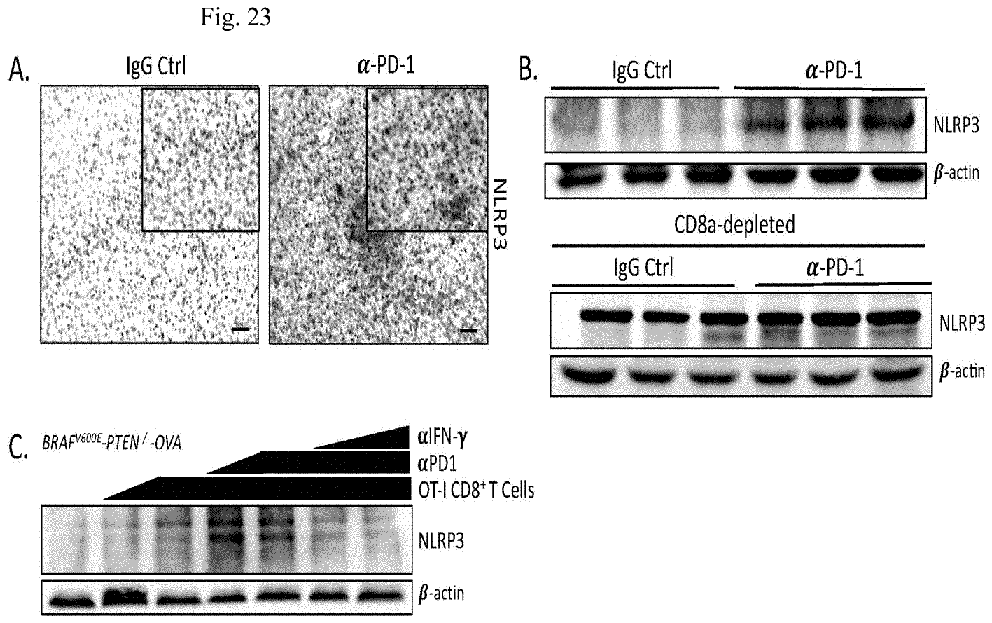

It is noteworthy that we also found BRAF^{V600E}PTEN^{-/-} melanomas to exhibit an elevation in NLRP3 expression in response to anti-PD-1 therapy both in vitro and in vivo, an effect that was enhanced by IFN-γ and reversed by the ablation of CD8⁺ T cells (FIG. 23) (19). This finding suggests that increased immunologic pressure may also select for tumors driven for more aggressive behavior through enrichment of tumor cell populations expressing higher levels of the NLRP3 inflammasome. These findings are reminiscent of those studies describing tolerogenic properties of longstanding exposure to IFN-γ and reveals a mechanism by which immunoediting may drive tumor escape and metastasis (39).

The HSP70-TLR4 Signaling Axis in Lung Epithelial Tissues Supports Primary Tumor Progression and Anti-PD-1 Immunotherapy Resistance

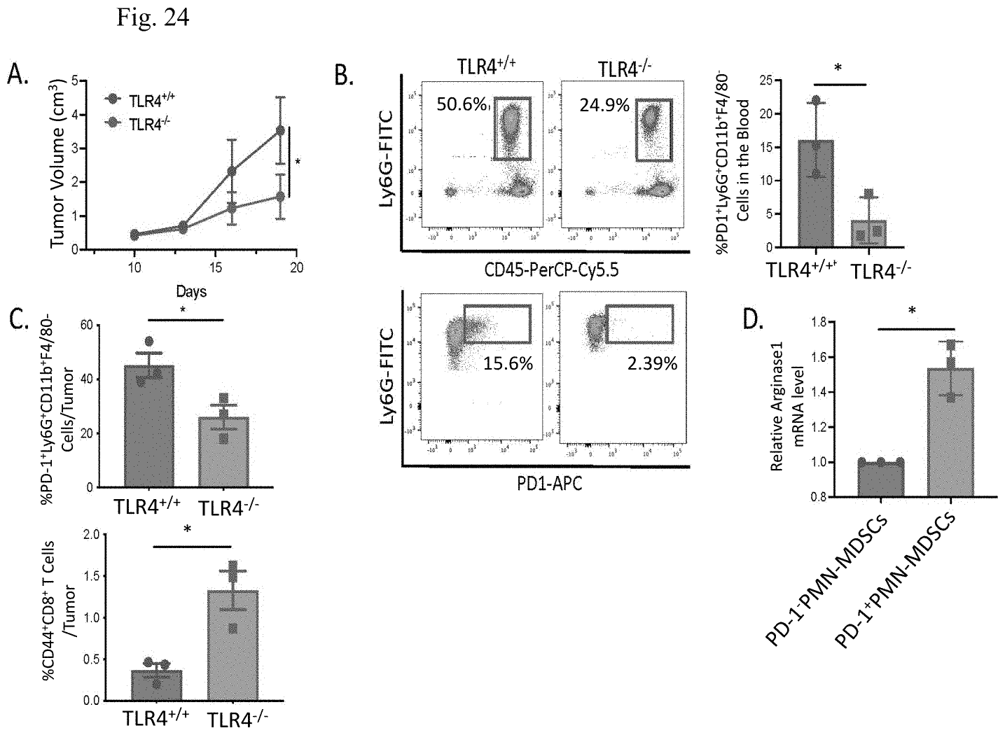

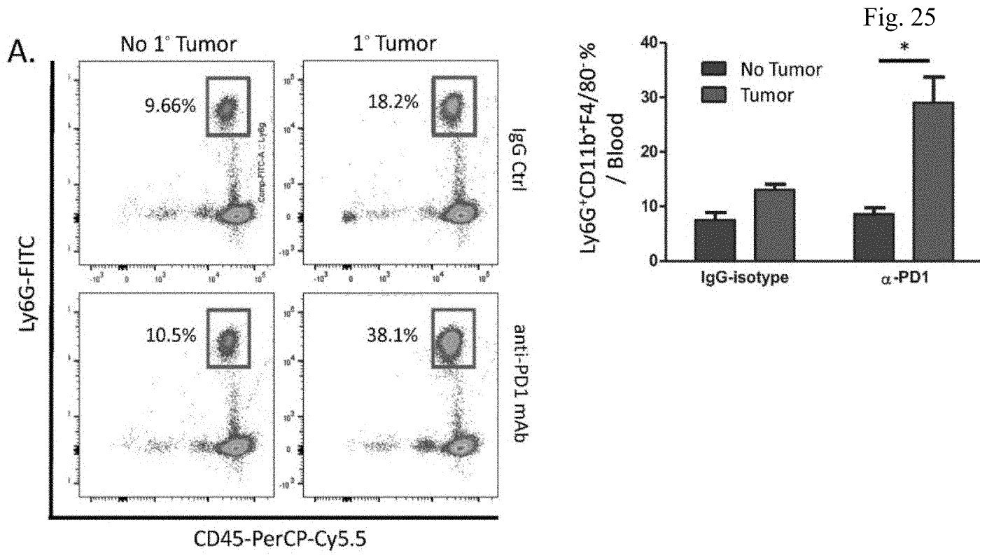

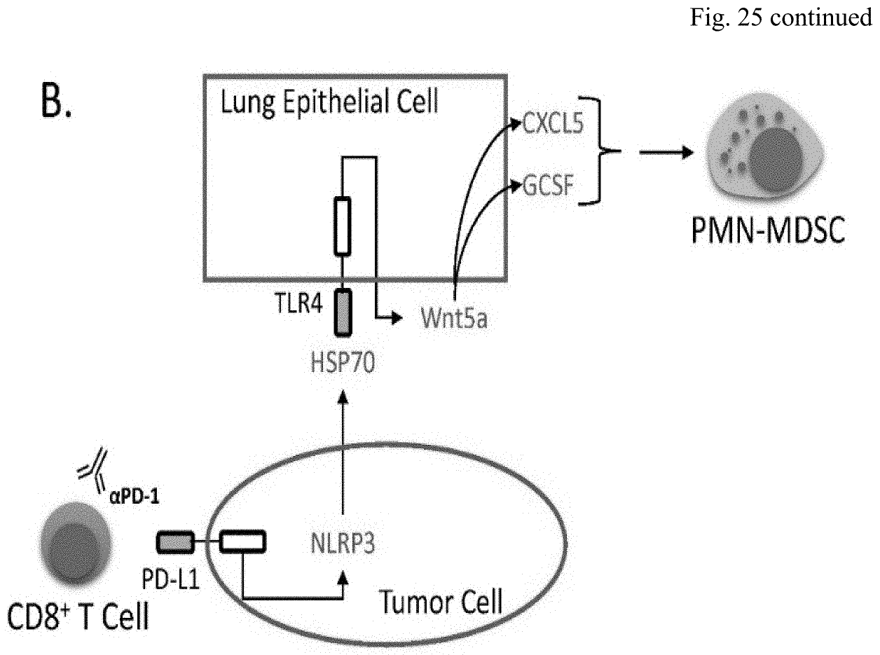

During the course of our studies, we found that primary melanomas responded more favorably to anti-PD-1 immunotherapy in SPC-TLR4^{-/-} hosts relative to TLR4^{fl/fl} control mice (FIG. 7A). This same observation was also made in the E0771 breast cancer model (FIG. 24, A to C). Based on multi-parameter flow cytometry, it was also noted that tumor-bearing TLR4^{fl/fl} control mice exhibit a significant increase in the number of circulating PD-1⁺CD45⁺CD11b⁺Ly6G⁺Ly6C^{lo}F4/80⁻ PMN-MDSCs in the blood relative to tumor-bearing SPC-TLR4^{-/-} hosts (FIGS. 7, B and C, and FIG. 24D). Granulocyte-colony stimulating factor (G-CSF) expression by the lung epithelium has been shown to promote neutrophilic inflammation and to induce PD-1 expression by MDSCs (40-42). Indeed, we found elevated G-CSF expression levels in the lung epithelium of tumor-bearing versus non-tumor-bearing hosts and we have observed significant increases in circulating PMN-MDSCs in tumor-bearing versus non-tumor-bearing mice, particularly in response to anti-PD-1 immunotherapy (FIG. 7D, FIG. 24B). We therefore hypothesized that the significant differences observed in the numbers of circulating PD-1⁺ PMN-MDSCs between SPC-TLR4^{-/-} and TLR4^{fl/fl} control mice are due to alterations in G-CSF expression in the lung epithelium, a finding that we confirmed based on both G-CSF-targeted qrt-PCR, IHC, and ELISA studies (FIG. 7, E to G). Since we have shown that tumor-derived HSP70 signals through TLR4 in the lung, we performed additional experiments showing that an antagonistic antibody to HSP70 also suppresses lung epithelial G-CSF expression levels (FIG. 7H). We previously demonstrated that TLR4 signaling can mediate Wnt5a upregulation in tumor cells and lung epithelial cells (FIG. 2D)(19). While we found rHSP70 to induce modest G-CSF upregulation, our in vitro studies indicate that Wnt5a is the more immediate driver of G-CSF in the lung epithelium (FIG. 7I). Together, these results suggest that, in addition to generating a CXCR2 chemokine gradient to attract PMN-MDSCs to lung tissues, the HSP70-TLR4-Wnt5a signaling axis also drives G-CSF-dependent granulopoiesis of PMN-MDSC populations from the bone marrow (FIG. 25B). We further speculate that the circulating PD-1⁺ PMN-MDSC population may also serve as a sink to eliminate anti-PD-1 antibodies from the circulation, thus diminishing the therapeutic efficacy of this agent and contributing to an overall immunotherapy resistant state.

Activation of the Tumor-Intrinsic NLRP3-HSP70 Axis is Associated with Hyperprogression in Melanoma Patients Undergoing Anti-PD-1 Immunotherapy

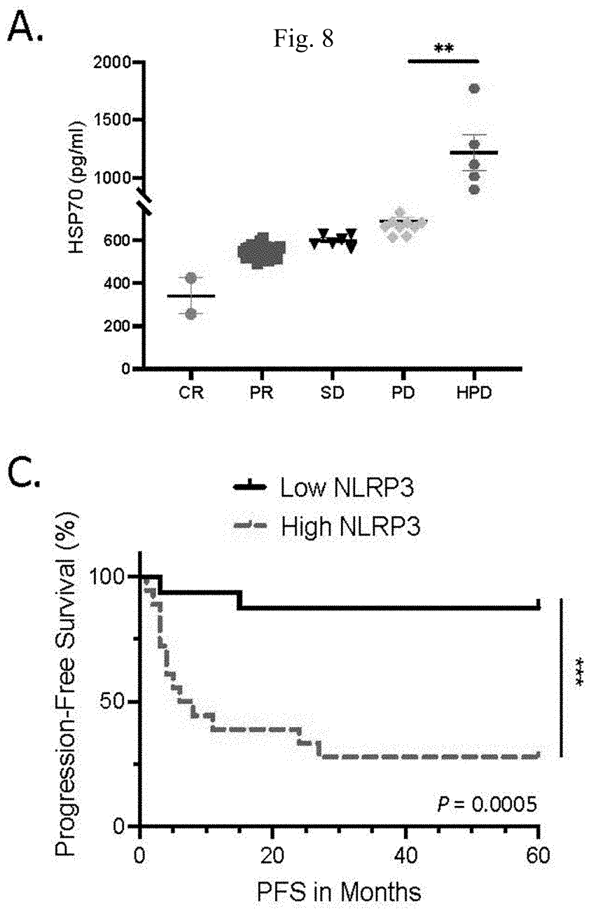

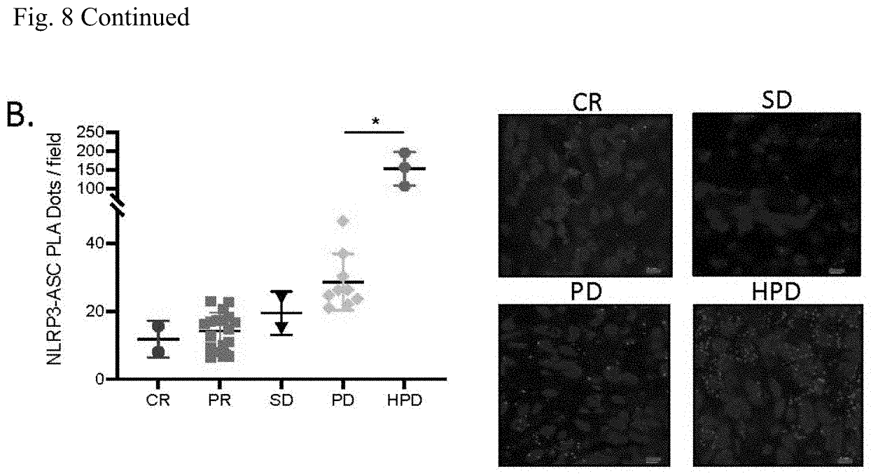

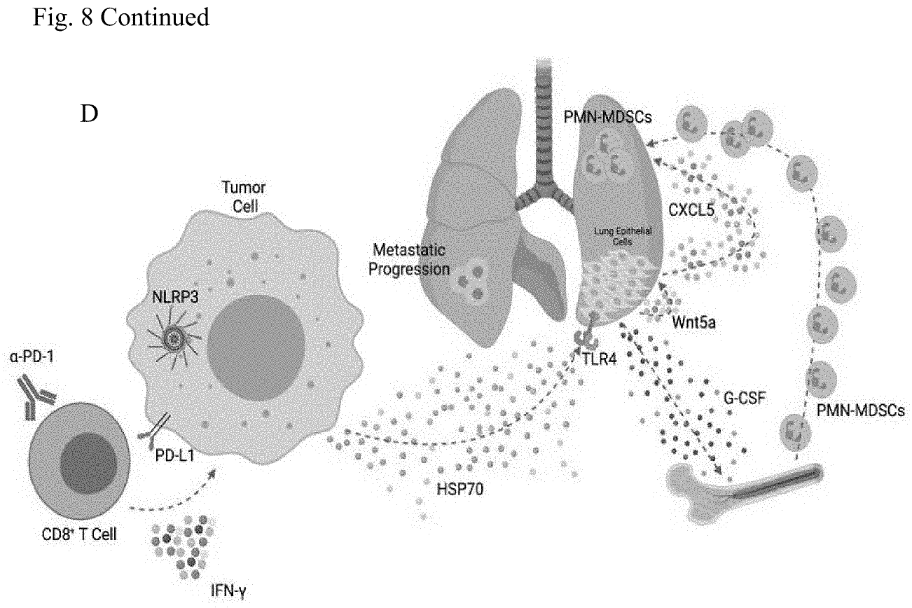

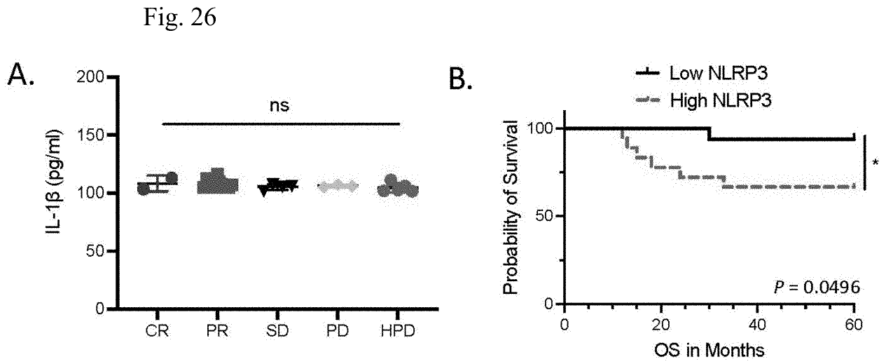

Based on our cumulative results suggesting a mechanistic link between the tumor intrinsic NLRP3-HSP70 axis and metastatic progression in response to anti-PD-1 immunotherapy, we measured baseline plasma HSP70 levels in advanced melanoma patients undergoing checkpoint inhibitor immunotherapy and evaluated these levels in terms of treatment response. While previous studies have defined HPD based on differential tumor growth rate (TGR) measurements before and after the initiation of checkpoint inhibitor therapy, prior imaging studies for many of our patients to define TGR prior to treatment initiation were not available. As a result, we utilized RECIST1.1 criteria to define HPD based on a two-fold increase in tumor burden within 2-3 months of anti-PD-1 initiation. This analysis demonstrated a significant increase in baseline HSP70 levels in those melanoma patients experiencing HPD while undergoing anti-PD-1 immunotherapy (FIG. 8A). Consistent with our prior data, we found no relationship between plasma IL-1β levels and HPD in this same cohort of melanoma patients (FIG. 26A). Based on these results, we sought to quantitate the activation level of the tumor-intrinsic NLRP3 inflammasome pathway in clinical tumor specimens to determine whether this may be an indicator of future behavior in response to anti-PD-1 immunotherapy. In order to achieve this goal, we utilized a PCR-based proximity ligation assay capable of identifying and quantitating NLRP3-ASC molecular interactions in FFPE-based tumor specimens as a surrogate for the activity level of the tumor NLRP3 inflammasome pathway (43). Using this approach, we were able to determine that tumors exhibiting evidence of enhanced NLRP3-ASC interactions at baseline ultimately developed HPD (FIG. 8B). This finding is consistent with the plasma HSP70 data and further indicates that plasma HSP70 levels are primarily a reflection of tumor-intrinsic NLRP3 activity. Further work also found that melanomas exhibiting enhanced NLRP3 activity based on the NLRP3-ASC PLA assay, defined as NLRP3-ASC interactions above the median, are associated with a significant reduction in progression-free (HR 0.12 (95% CI: 0.04-0.32)) and overall survival (HR 0.16 (95% CI: 0.04-0.70)) (FIG. 8C, and FIG. 26B). These studies in melanoma patients support the importance of the tumor NLRP3-HSP70 pathway in establishing a distant pre-metastatic niche via PMN-MDSC recruitment and indicate that this mechanism represents an important underlying driver of HPD in response to checkpoint inhibitor immunotherapy (FIG. 8D). Together, these findings suggest that quantitative measurements of activation of the NLRP3-HSP70 axis represents a promising strategy for predicting HPD in patients undergoing anti-PD-1 immunotherapy regimens. Future validation studies are warranted to confirm these findings in a larger cohort of melanoma patients as well as in other tumor types treated with PD-1-targeted agents.

Discussion

We previously described an adaptive resistance mechanism driven by the tumor-intrinsic NLRP3-HSP70 signaling axis that promotes the recruitment of PMN-MDSCs into the tumor microenvironment and suppresses local cytolytic CD8⁺ T cell activity in response to anti-PD-1 immunotherapy. We now demonstrate that the tumor-intrinsic NLRP3-HSP70 signaling axis can also induce the accumulation of PMN-MDSCs in distant tissues, thereby establishing a niche facilitating metastatic disease progression. Using an inducible lung epithelial cell-specific TLR4 knockout mouse model, we show this effect to be dependent upon tumor-derived HSP70 and its ability to trigger a distant TLR4-dependent Wnt5a-CXCL5/G-CSF signaling cascade capable of driving both PMN-MDSC granulopoiesis and recruitment into pulmonary tissues. Given that this mechanism is activated by anti-PD-1 immunotherapy, we conducted additional studies to determine whether this tumor NLRP3-HSP70-TLR4 axis could support the development of HPD. Indeed, pre-clinical tumor models of both melanoma and breast adenocarcinoma as well as clinical studies in advanced melanoma patients indicates that this pathway serves as a driver of HPD. While this work is consistent with previous findings that have implicated various myeloid cell populations as playing a critical role in promoting metastatic disease progression, it also highlights HSP70 as a previously undescribed soluble factor released by tumors that systemically drive PMN-MDSC accumulation (21). This relationship between circulating PMN-MDSCs or neutrophils and HPD has been described in previous studies although the underlying mechanisms linking these phenomena have not been elucidated (44). Just as we have shown HSP70 to signal through TLR4 to upregulate Wnt5a signaling via an autocrine tumor-intrinsic mechanism in a prior study, this current work shows circulating levels of HSP70 to induce the TLR4-Wnt5a-CXCL5/G-CSF signaling cascade in distant lung epithelial cells (19). These findings are also consistent with prior studies implicating TLR polymorphisms and signaling to support the establishment of pulmonary metastases (13, 45). These findings are interesting to consider in light of a recent retrospective study that found respiratory diseases and elevated neutrophil/lymphocyte ratios to be associated with disease progression in stage IV melanoma patients undergoing anti-PD-1 immunotherapy (46). Overall, these findings suggest that there is a continuum between mechanisms of adaptive resistance and disease hyperprogression in those patients undergoing checkpoint inhibitor immunotherapy.

While previous work has described several TDSFs capable of harnessing myeloid cell populations for pre-metastatic niche development in distant tissues, the underlying mechanisms driving the release of these factors have not been well described. Even less is understood regarding how therapeutic interventions modulate the release of TDSFs. Herein, we describe the underlying mechanism linking anti-PD-1 immunotherapy with tumor-dependent production of a TDSF capable of promoting metastatic progression in the lung via TLR4-dependent signaling. Whether this same mechanism can generate a pre-metastatic niche in other distant organ tissues requires further experimental investigation. To date, our data indicates that the tumor-intrinsic NLRP3 inflammasome stimulates this pathway in a similar manner across several different tumor types however additional studies based on clinical specimens are necessary to determine whether this same mechanism can also drive metastatic progression in other patient populations.

It is noteworthy that our work has identified two pathways by which the tumor NLRP3-HSP70-TLR4 pathway promotes the accumulation of PMN-MDSCs in distant tissues to establish the pre-metastatic niche necessary for metastatic progression. Indeed, tumor-dependent HSP70 drives the upregulation of both G-CSF and CXCL5 by triggering TLR4 signaling in the lung epithelium. Together, this mechanism drives the release of PMN-MDSCs into the circulation while also establishing a chemokine gradient capable of recruiting these PMN-MDSCs into pulmonary tissues. Interestingly, these studies also revealed this G-CSF-dependent mechanism to induce significant PD-1 upregulation by this circulating PMN-MDSC population, a finding suggesting the development of a potential sink for therapeutic anti-PD-1 antibodies and an additional mechanism by which the tumor NLRP3-HSP70-TLR4 axis can contribute to adaptive resistance to anti-PD-1 immunotherapy. Previous studies have implicated the accumulation of a c-kit⁺CD133⁺CD34⁺VEGFR1⁺ HPC population along with enhanced fibronectin expression in the extra-cellular matrix (ECM) as key components necessary for the evolution of the lung pre-metastatic niche (35). Interestingly, this current study further demonstrates that the tumor NLRP3 inflammasome as well as lung epithelial TLR4 signaling both support the accumulation of this HPC population in pulmonary tissues. Similar to a previous study linking Wnt5a with enhanced fibronectin expression in the lung, this study also shows that lung epithelial TLR4 as well as Wnt ligand signaling both contribute to fibronectin accumulation within the lung ECM (47). Together, these data indicate that the tumor NLRP3-HSP70-TLR4 axis represents an early step in establishing the pre-metastatic niche and therefore highlights this pathway as a target for preventing metastatic progression.

Prior studies have concluded that NLRP3 activation in melanoma cells elicits the secretion of the IL-1β pro-inflammatory cytokine (26, 27, 48). However, we have found the activation of the tumor-intrinsic NLRP3 inflammasome to promote much higher levels of HSP70 secretion relative to IL-1β based on both in vitro and in vivo studies in mice and humans. Indeed, our work indicates that IL-1β secretion in response to NLRP3 activation is more prominent relative to HSP70 in myeloid cells such as dendritic cells, suggesting that the HSP70 mediator is more specific to tumor NLRP3 inflammasome activity relative to IL-1β. These observations indicate that HSP70 represents a promising pharmacologic target for suppressing metastatic progression and enhancing anti-tumor immunity. This is consistent with our pre-clinical data demonstrating that an antagonistic antibody specific to HSP70 robustly suppresses PMN-MDSC levels in tumor tissues and inhibits disease progression in a treatment refractory autochthonous model of melanoma.

In addition to characterizing how the tumor-intrinsic NLRP3 inflammasome pathway contributes to metastatic disease progression and demonstrating that this process is driven by PD-1 blockade in various pre-clinical tumor models, we also present data illustrating that quantitative measures of this pathway correlate with disease HPD in advanced melanoma patients undergoing anti-PD-1 immunotherapy. After defining HPD in our patient cohort as a two-fold increase in overall tumor burden by week 12 of therapy, we found that baseline plasma levels of HSP70 were significantly elevated in HPD patients relative to responding patients as well as those patients with stable or progressive disease without evidence of hyperprogression. Importantly, we then interrogated melanoma tissue specimens using a proximity ligation assay capable of quantifying NLRP3-ASC binding as a surrogate for activation of the NLRP3 inflammasome. This approach also verified that those tumors exhibiting enhanced levels of NLRP3 activation at baseline developed HPD following the initiation of anti-PD-1 immunotherapy. Whiles these studies require validation in a larger cohort of patients, these findings 1) further substantiate the NLRP3-HSP70 signaling pathway as an important driver of HPD in response to anti-PD-1 immunotherapy and 2) indicate that assays to quantitate the level of NLRP3-HSP70 signaling activation can be employed to identify those patients at risk for developing HPD as a complication of anti-PD-1 checkpoint inhibitor immunotherapy. While the definition and incidence of HPD associated with anti-PD-1 immunotherapy continues to be debated, it clearly represents a devastating side-effect of our immunotherapy arsenal. By providing an underlying mechanism responsible for driving HPD, these areas of controversy can be resolved by further clinical studies in melanoma as well as other solid tumors.

It is tempting to speculate that genetic alterations that serve to enhance the activity of the tumor-intrinsic NLRP3 inflammasome will promote the described adaptive resistance mechanism and perhaps drive HPD in response to checkpoint inhibitor immunotherapies (49, 50). In light of the percentage of tumors exhibiting NLRP3 amplification, this genetic alteration may contribute to the development of this phenotype in various solid tumors treated with anti-PD-1 immunotherapy (38). Our pre-clinical modeling studies presented here have confirmed that this mechanism could contribute to such a response to checkpoint inhibitor immunotherapy. Whether other somatic or germline mutations involving NLRP3 itself or its various regulators may also enhance the activation of this pathway in response to checkpoint blockade remains unknown but is currently under investigation.

Together, this work provides insight into the importance of the tumor-intrinsic NLRP3-HSP70 signaling axis in regulating PMN-MDSCs within distant tissues while also highlighting its critical role in modulating responses to immunotherapy. Additional studies are now ongoing to 1) better understand the regulation of this pathway in tumors and how these mechanisms may dictate how a tumor responds to anti-PD-1 immunotherapy and 2) determine whether targeting HSP70 may be an effective strategy for overcoming resistance to anti-PD-1 immunotherapy. Overall, these data describe an adaptive resistance mechanism to anti-PD-1 immunotherapy capable of supporting HPD in select settings. Future clinical studies are warranted to test whether markers associated with the NLRP3-HSP70 signaling axis can be used to predict those melanoma patients at risk for developing HPD in response to anti-PD-1 immunotherapy while also investigating the role of the tumor NLRP3-HSP70 signaling axis in other cohorts of cancer patients.

Materials and Methods

Study Design

The primary objective of this work was to investigate the role of the tumor-intrinsic NLRP3 inflammasome and its downstream effector, HSP70, in PMN-MDSC-mediated pre-metastatic niche development in distant tissues and to determine whether this pathway may also contribute to the development of HPD in response to checkpoint inhibitor immunotherapy. The overall goal was to identify novel pharmacologic targets as well as biomarkers capable of improving our ability to detect those patients at risk for developing HPD and to improve our management of this complication associated with checkpoint inhibitor immunotherapy. The study included laboratory-controlled in vitro cell culture experiments, in vivo animal experiments, studies utilizing clinical specimens derived from stage IV melanoma patients undergoing anti-PD-1 immunotherapy, as well as in silico analysis of an existing tumor tissue database. The impact of the tumor-intrinsic NLRP3 inflammasome on 1) PMN-MDSCs was measured based on both flow cytometry, cytology, IHC, IF, and qrt-PCR analysis, 2) tumor progression and metastasis was measured based on primary tumor size measurements, primary tumor weight, lung weight, H&E microscopy, qrt-PCR, IHC, cell proliferation, and cell invasion assays. Activation of the NLRP3 pathway was measured using Western blot, qrt-PCR, ELISA, and a NLRP3-ASC PLA. Mice were treated with pharmacologic inhibitors of the NLRP3 inflammasome and HSP70 while PMN-MDSCs were ablated by antibody-dependent cellular cytotoxicity. The NLRP3-HSP70 signaling axis was manipulated using both CRISPR/Cas9 and CRISPRa while TLR4 was genetically deleted specifically in lung epithelial cells and NLRP3 was deleted systemically using transgenic mouse systems. For animal experiments, an even distribution of male and female mice was randomly assigned into treatment groups. Sample size was determined based on an alpha probability of 0.05, a power of 0.8 and an effect of at least 1.2×SD. Preliminary in vivo studies have shown that sample sizes need to be at least 6 or greater per group in syngeneic tumor model systems while 8 or greater are necessary in autochthonous tumor model systems. Notably, fewer sample numbers per group were found to be sufficient depending on analysis endpoints. All experiments were performed independently at least three times. All experiments were conducted in a blinded fashion where analysis was independent of any intervention when feasible. All outliers have been included in the data presented.

Clinical Samples

All patients provided written informed consent for use of biological specimens on an ongoing institutional review board-approved clinical specimen acquisition protocol at Duke Cancer Institute designed to investigate immunotherapy resistance (NCT02694965). Baseline (week 0) FFPE tissues were collected from 35 untreated stage IV melanoma patients and baseline plasma samples were collected from 80 untreated stage IV melanoma patients prior to initiating anti-PD-1 monotherapy at Duke Cancer Institute. Treatment response at week 12 and every 12 weeks thereafter was determined based on independent radiologic review of computed tomography (CT) imaging using RECIST1.1 criteria. HPD was defined as a ≥2-fold increase in total tumor burden by week 12 CT imaging.

Animal Studies

C57BL/6J (C57, H-2^{b}) (Stock number: 000664), B6.CgBraf^{tm1Mmcm}Pten^{tm1Hwu}Tg(Tyr-cre/ERT2)13Bos/BosJ (Braf^{V600E}Pten^{-/-}, H-2^{b}) (Stock number 012328), B6(Cg)-Tlr4^{tm1.2Karp}/J(TLR4^{fl/fl}; Stock number: 24872), B6.129S-Sftpc^{tm1(cre/ERT2)Blh}/J(SPC-CreER^{T2}) (Stock number: 28054) and C57BL/6Tg(TcraTcrb)1100Mjba (OT-1, H-2^{b}) (Stock number: 003831) mice were obtained from Jackson Labs. Dr. Mari Shinohara (Duke University) generously provided B6.129S6-Nlrp3^{tm1Bhk}/J(NLRP3KO) mice. SPC-Cre-ER^{T2} mice were crossed with TLR4^{fl/fl} mice to generate SPC-Cre-ER^{T2}/TLR4^{fl/fl} offspring (SPC-TLR4^{-/-}). Conditional knock-out of Tlr4 was confirmed via PCR per protocol provided by Jackson Laboratory as well as flow cytometry. Mice were treated with 100 μl tamoxifen (Sigma-Aldrich, CAS #10540-29-1, 20 mg/mL) via i.p. delivery daily×5 consecutive days for a total dose of 75 mg/kg (51). All experimental groups included randomly chosen littermates of both sexes, ages 6-10 weeks, and of the same strain. All animal experiments were performed based on a protocol approved by the Institutional Animal Care and Use Committee at Duke University Medical Center.

Autochthonous Tumor Studies

B6.Cg-Braf^{tm1Mmcm} Pten^{tm1Hwu} Tg(Tyr-cre/ERT2 H-2^{b})13Bos/BosJ transgenic mice were subdermally injected with 4-Hydroxytamoxifen (4-HT) (Sigma, H6278-50MG CCF, 38.75 μg/mouse) to induce primary melanoma development at the base of the tail. Mice were randomly assigned to treatment cohorts until tumor volumes reached 64 mm³ (36, 37, 52). For select experiments, mice were treated with the following agents: NLRP3 inhibitor, MCC950 (Invivogen, inh-mcc) 10 mg/kg intra-peritoneal injection (i.p.) every other day, anti-PD1 ab (BioXCell, BE0146) or rat IgG2a isotype control (BioXCell, BE0089) at 200 μg i.p. every 3 days, anti-Ly6G ab (BioXCell, BE0075-1), initial dose at 200 μg/mouse followed by 100 μg/mouse/day×2, HSP70 monoclonal antibody (3A3, MA3-006) at 5 μg i.p. every 3 days. Primary tumor volumes were monitored by orthogonal caliper measurements every 3 days. Tumor volume was calculated according to the formula: cm³=[(length, cm)×(width, cm)²]/2.

Syngeneic Tumor Studies.

BRAF^{V600E}PTEN^{-/-}, BRAF^{V600E}PTEN^{-/-}NTC, BRAF^{V600E}PTEN^{-/-}-NLRP3^{KD}, BRAF^{V600E}PTEN^{-/-}-NLRP3a, BRAF^{V600E}PTEN^{-/-}-Ctrl, BRAF^{V600E}CDKN2A^{-/-}PTEN^{-/-} cell lines (0.5×10⁵ to 1×10⁶ cells) were implanted by s.c. injection into the base of the tail or chest of syngeneic C57BL/6 mice. The E0771 cell line (0.25×10⁶ to 0.5×10⁶) was injected into the mammary fat pad of syngeneic C57BL/6 mice in 0.1 mL sterile saline using a 27G needle. Tumor growth was monitored by caliper measurement every 3 days, and treatment was initiated when tumor volumes reached 64 mm³ or 90 mm³ depending on the study. Once tumor volume reached 500-600 mm³ in select experiments, mice were anesthetized using 2% isoflurane via an anesthesia mask and primary tumor tissues were resected as previously described (53). Wounds were managed with surgical clips and an antiseptic iodine solution was applied to the site. Following surgery, animals were monitored under a heat lamp until fully recovered.

Cell Lines and Culture Conditions

BRAF^{V600E}PTEN^{-/-}-NLRP3a and BRAF^{V600E}PTEN^{-/-}-Ctrl cell lines were generated using the established CRISPR amplification technique (54). The NLRP3 and control CRISPR activation plasmids (SantaCruz, sc-432122-ACT) were packaged into a lentiviral vector in HEK293T cells as previously prescribed (55). Clones were selected using a combination of antibiotics including puromycin (SantaCruz, sc-108071), hygromycin B (SantaCruz, sc-29067), and blasticidin S HCL (SantaCruz, sc-495389). BRAF^{V600E}PTEN^{-/-}-NLRP3^{KD} and BRAF^{V600E}PTEN^{-/-}-NTC cell lines were generated as described previously (19). The BRAF^{V600E}PTEN^{-/-} (male, BPD6) cell line was also generated previously (36). Stable cell lines were selected by puromycin resistance (Sigma-Aldrich, P8833). MLE12(CRL-2110) and BRAF^{V600E}CDKN2A^{-/-}PTEN^{-/-} (YUMM1, CRL-3363) cell lines were purchased from ATCC. The EO771 mammary tumor cell line was generously provided by Dr. Donald McDonnell (Duke University). All BRAF^{V600E}PTEN^{-/-} cell lines and E0771 were maintained at 37° C. in DMEM (Invitrogen) with 2 mM L-glutamine, supplemented with 10% fetal bovine serum (FBS), 100 units/ml penicillin. MLE12 cell line was maintained in HITES medium supplemented with 2% FBS. For select in vitro experiments, cell lines were treated with Wnt5a (100-200 ng/ml, R&D Systems/Bio-techne, 645-WN-010), IFNγ (100 ng/ml, BioAbChem, 42-IFNg), anti-PD-L1 ab (1-2 μg/ml, TLR4 inhibitor (3 μM-10 Invivogen, tlrl-cli95) and MCC950 NLRP3 inhibitor (2.5 μM-10 Invivogen, inh-MCC). All cell lines were tested Mycoplasma-free by Duke University Cell Culture Facility shared services.

Tumor Cell-CD8+ T Cell Co-Culture Assays and CD8⁺ T Cell Proliferation Assays

Naïve CD8⁺ T cells were isolated from the spleens of OT-1 transgenic mice by magnetic bead CD8 purification according to the manufacturer's instructions (Miltenyi Biotec, 130-104-075) and activated with IL-2 (100 U/ml, PeproTech, 212-12) and SIINFEKL peptide (1 μg/ml, New England Peptide, BP10-915) for 3 days. Activated OT-1 CD8⁺ T cells were incubated with BRAF^{V600E}PTEN^{-/-}-OVA cells in the presence and absence of anti-PD1 ab (1 μg/ml) and/or IFNγ ab (MAB4851, 4-20 ng/mL) for 72 hrs at a tumor cell:CD8⁺ T cell ratio of 1:5. For the ex vivo CD8⁺ T cell proliferation assay, splenocytes were isolated from IgG isotype control- and anti-Ly6G ab-treated mice. Then single cells were labeled with CFSE (ThermoFisher, C34554) and cultured in 96-well flat-bottom plates for either 3 or 6 days at 37° C. in RPMI medium supplemented with 10% FBS, penicillin and streptomycin. Cells were harvested and activated CD8⁺ T cells were quantitated based on flow cytometry. For in vitro PMN-MDSC suppression T cell proliferation assays, naïve CD8⁺ T cells were purified from lymph nodes, labeled with CFSE, stimulated with anti-CD3/anti-CD28 beads (Thermo fisher, 1116D) by 1:1 bead to cell ratio, in the presence or absence of PMN-MDSCs (1:3 T cell/PMN-MDSC ratio) for 4 days. Cells were harvested on day 4 and activated CD8⁺ T cells were quantitated based on flow cytometry.

Cell Invasion Assay

A Matrigel invasion chamber with an 8 um transwell membrane (CORNING, 354480) was utilized to examine the relative invasion properties of cell lines. BRAF^{V600E}PTEN^{-/-}-NLRP3a and BRAF^{V600E}PTEN^{-/-}-Ctrl cell lines (5×10⁴ cells) seeded in the upper chamber in serum-free conditions while FBS was added to the lower chamber. After 48 hrs, transwell inserts were washed and cells were fixed with methanol and stained with crystal violet (1% w/v) for 10 mins before microscopic quantification.

Tumor and Lung Tissue Cell Isolation

Tumors were resected at a similar size and mechanically disaggregated by a gentleMACS dissociator (Miltenyi) and then digested with serum-free RPMI containing collagenase IV (1 mg/mL, Sigma-Aldrich), hyaluronidase (0.1 mg/mL, Sigma-Aldrich), and deoxyribonuclease (20 U/mL, Sigma-Aldrich) on a shaker at 250 rpm at 37° C. for 30 mins (36). Resected lung tissues were mechanically separated by gentleMACS (lung mode) and enzymatically digested using the same enzyme digestion solution and the same conditions for 20 mins. Cell suspensions were filtered using a 70 μm filter, washed with flow buffer (PBS, 2 mM EDTA, 2% FBS/BSA), and red blood cells were lysed using lysis buffer (Sigma) for 10 mins at room temperature (RT).

RNA Isolation and Qrt-PCR Analysis

Small representative tissue specimens were harvested from tumors and lungs and stored in RNAlater at −80° C. Tissues were lysed in RLT buffer by using a gentleMACS dissociator (Miltenyi) while cell lines were lysed in RLT buffer and stored at −80° C. Lung tissue-derived cells were sorted by flow cytometry based on their surface marker expression and lysed in RLT buffer before storing at −80° C. Total RNA was isolated by RNeasy Plus Mini Kit (Qiagen, 74134) and RNA was quantified by NanoDrop. RNA (500 ng-1000 ng) was used in cDNA synthesis (iScript Reverse Transcription Supermix, BioRad, 1708841). Real-time PCR was performed using an ABI7500 Real-Time PCR system (Life Technologies). All qPCR reactions were performed using validated primers and SsoAdvanced Universal SYBR Green Super Mix (BioRad, 1725271) or SsoAdvance Universal Probes Supermix (BioRad, 1725281). For developing a TRP2 qrt-PCR assay to measure melanoma metastases in lung tissues, 1×10²-1×10⁶ BRAF^{V600E}PTEN^{-/-} melanoma cells were admixed with a single cell suspension generated from a single lung lobe and processed for qrt-PCR analysis using Trp2-specific TaqMan probes. Relative Trp2 mRNA levels were correlated with the number of admixed melanoma cells and both high sensitivity and specificity were achieved. All data were normalized to Actb expression and relative gene expression was quantitated based on the 2ΔΔCt method. Relative mRNA levels of the following genes were measured: Cxcl5, Cxcl1, Cxcl2, Trp2, Csf3, Il1b, Fn1, Tgfb, Tgfbr2, Spp1, and Hsp70.

Western Blot Analysis

Tissues or cells were lysed in NP40 lysis buffer (Sigma-Aldrich) supplemented with a complete protease inhibitor and phosphatase inhibitor (Roche). After lysing in Laemmli sample buffer, equal volumes of lysates were separated using 10% or 15% SDS-PAGE and then transferred to a PVDF membrane (Bio-Rad Laboratories Inc., 162017). After blocking for 30 mins in tris-buffered saline containing 0.1% Tween-20 and 5% milk, the membranes were probed with various primary antibodies followed by HRP-conjugated secondary antibodies. Primary antibodies included: Anti-β-actin mouse monoclonal (clone c4, 1:3000, Santa Cruz Biotechnology, sc-47778), anti-NLRP3 rabbit monoclonal (clone D4D8T, 1:1000, Cell Signaling, 15101S; clone EPR23073-96, 1:1000, Abcam, 270499), anti-Caspase-1-p20, mouse monoclonal (clone Casper-1, 1:500, Adipogen, AG-20B-0042-C100), anti-HSP70, mouse monoclonal (clone C92F3A-5, 1:1000, Santa Cruz Biotechnology, sc-66048), anti-CXCL5, goat polyclonal (clone P50228, 1:1000, R&D Systems, AF433), anti-Wnt5a, mouse monoclonal (clone A5, 1:1000, Santa Cruz Biotechnology, sc-365370), anti-G-CSF, rabbit monoclonal (clone ABAC-3, M02280-1, Bosterbio), and anti-IL-1-β, mouse monoclonal (clone 3A6, 1:1000, Cell Signaling, 12242). Immunoreactivity was visualized using chemiluminescence substrate (ThermoFisher, 34095/34075) and imaged by a ChemiDoc XRSplus System (BioRad).

ELISA

CXCL5, HSP70, GCSF and IL-1β concentrations in culture supernatants and mouse plasma samples were determined by appropriate mouse ELISA kits (CXCL5: R&D Systems, MX000; HSP70: R&D Systems, DYC1663-2; G-CSF: R&D Systems, DY414-05; IL-1β: R&D Systems, DY401-05) according to the manufacturer's instructions. Human plasma HSP70 and IL-1β concentrations were measured using the human DuoSet assay (R&D Systems, HSP70: DY1663, IL-1β: DY201-05) according to manufacturer's protocol.

Flow Cytometry Analysis

Cells (1-2×10⁶) were diluted in 150 μl flow buffer per well of V bottom culture plates, incubated with Live/Dead Stain (Sigma) on ice for 20 mins, washed twice, and incubated with Fc Block (anti-CD16/CD32, 2 ug/mL) before staining with appropriate conjugated antibodies. Cells were washed, resuspended in 2% paraformaldehyde for 15 mins at 4° C., and analyzed using a BD FACSCanto flow cytometry system. Compensation was performed using (FMO) controls. Cells were characterized using the following combinations of cell surface markers after gating on viable single cell populations. All antibodies were obtained from commercial vendors: Anti-Mouse CD11b, PE-conjugated, clone: MIH5 (BD Pharmingen, 558091), Anti-Mouse Ly6G-GR1, FITC conjugated, clone: RB6-8C5 (BD Pharmingen, 5532127), Anti-Mouse Ly6G, FITC conjugated, clone: 1A8 (BioLegend, 127605), Anti-mouse Ly-6G Antibody, FITC conjugated, clone 1A8-Ly6g, (ThermoFisher, 11-9668-82), Anti-mouse F4/80 Antibody, APC conjugated, clone: BM8 (BD Pharmingen, 560408), Anti-Mouse CD45, PerCP-Cy5.5 conjugated, clone: 145-2C11 (BD Pharmingen, 551163), Anti-mouse CD326/Ep-CAM antibody, FITC conjugated, clone: G8.8, (Bio Legend, 118207), Anti-mouse CD326, APC conjugated, clone: G8.8 (BD Bioscience, 563478), Anti-mouse CD90.2 antibody, FITC conjugated, clone: 53-2.1, (BD Bioscience, 553003), Anti-Mouse CD8a, BV510 conjugated, clone: 53-6.7 (BD Pharmingen, 563068), Anti-Mouse CD3e, PerCP-Cy5.5 conjugated, clone: 145-2C11 (BD Pharmingen, 551163), Anti-Mouse CD8a, APC conjugated, clone: 53-6.7 (BD Bioscience, 553035), Anti-Mouse PD1/CD279, PE-conjugated, clone: 29F.1A12 (BD Bioscience, 568250), and Anti-Mouse Ly6C, PE-Cy7 conjugated, clone: AL-21 (BD Pharmingen, 560593), Anti-Mouse VEGFR1, PE-conjugated, clone: 141522 (Novus Biologicals, FAB4711P), Anti-Mouse CD133, PE-Cy7-conjugated, clone: 315-2C11 (BioLegend, 141209), Anti-Mouse CD34, FITC-conjugated, clone: RAM34 (BD Bioscience, 560238), and Anti-Mouse CD117/C-KIT, PerCP-Cy5.5-conjugated, clone: 2B8 (BD Bioscience, 560557). Cell populations were characterized based on the following marker profiles: Ly6G+ PMN-MDSCS cells: CD45+CD11b+Ly6G^{hi}Ly6C^{lo} F4/80⁻ Tumor-associated Macrophages: CD45+CD11b+Ly6G−F4/80+; Type II Lung Epithelial Cells: CD45⁻CD90.2⁻EPCAM⁺; CD8⁺ T Cells: CD45⁺CD3e⁺CD8a⁺CD44^{+/-}; bone marrow-derived hematopoietic cells: c-kit⁺VEGR1⁺CD133⁺CD34⁺. Flow cytometry data was analyzed using Flowjo software v10.3.

Immunohistochemistry and Immunofluorescence Analysis

Paraffin-Sections (5-μm) from primary melanomas and lung tissues were processed using standard protocols for immunohistochemistry (IHC) and immunofluorescence (IF) staining. Tissues was permeabilized by incubation in 0.4% Triton-X in TBS for 20 min. The following primary antibodies were used: anti-Wnt5a, mouse monoclonal (clone: A-5, 1:200, Santa Cruz, 365370), anti-Ly6G, rabbit monoclonal (clone: EPR22909-135, 1:100, Abcam, ab238132), S100beta, rabbit polyclonal (clone: 6285, 1:500/1:250, Novus biological, NBP1-87102), anti-NLRP3, rabbit polyclonal (clone: 114548, 1:500, Novus biological, NBP2-12446), anti-GCSF, rabbit monoclonal (clone: ABAC-3, M02280-1), anti-Fibronectin, mouse monoclonal (clone: TV-1, NBP2-32849), and rabbit-specific HRP/AEC IHC Detection Kit-Micro-polymer (Abcam, ab236468). Anti-rat polymers were used as secondary antibodies and chromogen detection system for immunohistochemistry. For IF, goat anti-rabbit conjugated to AlexaFluor564 and goat anti-mouse Alexa488 were used as secondary antibodies for the appropriate primary antibody. Sections were imaged with an Axio Imager upright microscope. PMN-MDSC quantitation by Ly6G-staining, and CD8⁺ T cell quantitation by CD8a-staining were performed at 20× magnification and 6-8 random fields were averaged per section over 3-4 sections per specimen. Area calculations for lung tumor burden based on S100β-staining was performed at 20× magnification of 2-3 sections per specimen and quantified using ImageJ software (tumor burden area/total lung area).

Proximity Ligation Assay

Human tumor tissues (5 μm) were deparaffinized, rehydrated, and subjected to antigen retrieval using standard procedures. Tissues were permeabilized by incubation in 0.4% Triton-X in TBS for 20 min, incubated with a blocking solution containing 0.1% BSA in TBS, and 0.05% Tween solution for 30 min at RT. Slides were then incubated overnight with a cocktail of human NLRP3/NALP3 (aa 540-689) antibody (R&D Systems, AF7010) and anti-human ASC, mouse monoclonal antibody (Santa Cruz Biotechnology, sc-514414) at 4° C. Then a mixture of 1× DuoLink in situ PLA probe anti-mouse PLUS and 1× DuoLink PLA probe anti-goat MINUS (Sigma-Aldrich, DU092001-30RXN) was added to the section and incubated for 1 h in a humidity chamber preheated to 37° C. Ligase solution is added to each sample and incubated for an additional 30 min in a humidity chamber at 37° C. Amplification solution (35 μl) was added to each slide and incubated for 100 min in the humidity chamber at 37° C. Finally, mounting medium with DAPI was added and a cover slip was placed. Images were taken by a SP5 Leica confocal microscope. ImageJ software was used to quantify fluorescent spots in 3 fields per tissue section at 40× magnification and averaged over 2 tissue sections per tissue sample.

TCGA Data Analysis

NLRP3 amplification was quantitated in different cancer types using the TCGA database. Data was visualized using cBioPortal.

Statistics

Specific statistical tests are reported in the Figure Legends. GraphPad Prism 9 Windows version was used for all statistical analyses. Unpaired t-test was used to compare mean differences between control and treatment groups. One- or two-way ANOVAs followed by Tukey's multiple comparisons test or Sidak's multiple comparisons test, respectively, were performed to analyze data containing three or more groups. Progression-free and overall survival in stage IV melanoma patients undergoing anti-PD-1 immunotherapy was analyzed using a log-rank test. A P value of less than 0.05 was considered significant. All quantitative data are presented as the mean±SEM.

Study Approval

Mouse tumor experiments were performed according to a protocol approved by the IACUC of Duke University. All stage IV melanoma patients provided written informed consent under approval from the Institutional Review Board at Duke University (NCT02694965).

### REFERENCES

1. Y. Liu, X. Cao, Characteristics and Significance of the Pre-metastatic Niche. Cancer Cell 30, 668-681 (2016).
2. H. Peinado, H. Zhang, I. R. Matei, B. Costa-Silva, A. Hoshino, G. Rodrigues, B. Psaila, R. N. Kaplan, J. F. Bromberg, Y. Kang, M. J. Bissell, T. R. Cox, A. J. Giaccia, J. T. Erler, S. Hiratsuka, C. M. Ghajar, D. Lyden, Pre-metastatic niches: organ-specific homes for metastases. Nat Rev Cancer 17, 302-317 (2017).
3. S. Champiat, R. Ferrara, C. Massard, B. Besse, A. Marabelle, J. C. Soria, C. Ferte, Hyperprogressive disease: recognizing a novel pattern to improve patient management. Nat Rev Clin Oncol 15, 748-762 (2018).
4. R. Ferrara, L. Mezquita, M. Texier, J. Lahmar, C. Audigier-Valette, L. Tessonnier, J. Mazieres, G. Zalcman, S. Brosseau, S. Le Moulec, L. Leroy, B. Duchemann, C. Lefebvre, R. Veillon, V. Westeel, S. Koscielny, S. Champiat, C. Ferte, D. Planchard, J. Remon, M. E. Boucher, A. Gazzah, J. Adam, E. Bria, G. Tortora, J. C. Soria, B. Besse, C. Caramella, Hyperprogressive Disease in Patients With Advanced Non-Small Cell Lung Cancer Treated With PD-1/PD-L1Inhibitors or With Single-Agent Chemotherapy. JAMA Oncol 4, 1543-1552 (2018).
5. S. Champiat, L. Dercle, S. Ammari, C. Massard, A. Hollebecque, S. Postel-Vinay, N. Chaput, A. Eggermont, A. Marabelle, J. C. Soria, C. Ferte, Hyperprogressive Disease Is a New Pattern of Progression in Cancer Patients Treated by Anti-PD-1/PD-L1. Clin Cancer Res 23, 1920-1928 (2017).
6. S. Kato, A. Goodman, V. Walavalkar, D. A. Barkauskas, A. Sharabi, R. Kurzrock, Hyperprogressors after Immunotherapy: Analysis of Genomic Alterations Associated with Accelerated Growth Rate. Clin Cancer Res 23, 4242-4250 (2017).
7. T. Kamada, Y. Togashi, C. Tay, D. Ha, A. Sasaki, Y. Nakamura, E. Sato, S. Fukuoka, Y. Tada, A. Tanaka, H. Morikawa, A. Kawazoe, T. Kinoshita, K. Shitara, S. Sakaguchi, H. Nishikawa, PD-1(+) regulatory T cells amplified by PD-1 blockade promote hyperprogression of cancer. Proc Natl Acad Sci USA 116, 9999-10008 (2019).
8. D. H. Kang, C. Chung, P. Sun, D. H. Lee, S. I. Lee, D. Park, J. S. Koh, Y. Kim, H. S. Yi, J. E. Lee, Circulating regulatory T cells predict efficacy and atypical responses in lung cancer patients treated with PD-1/PD-L1 inhibitors. Cancer Immunol Immunother, (2021).
9. G. Lo Russo, M. Moro, M. Sommariva, V. Cancila, M. Boeri, G. Centonze, S. Ferro, M. Ganzinelli, P. Gasparini, V. Huber, M. Milione, L. Porcu, C. Proto, G. Pruneri, D. Signorelli, S. Sangaletti, L. Sfondrini, C. Storti, E. Tassi, A. Bardelli, S. Marsoni, V. Toni, C. Tripodo, M. P. Colombo, A. Anichini, L. Rivoltini, A. Balsari, G. Sozzi, M. C. Garassino, Antibody-Fc/FcR Interaction on Macrophages as a Mechanism for Hyperprogressive Disease in Non-small Cell Lung Cancer Subsequent to PD-1/PD-L1 Blockade. Clin Cancer Res 25, 989-999 (2019).
10. I. Matos, J. Martin-Liberal, A. Garcia-Ruiz, C. Hierro, M. Ochoa de Olza, C. Viaplana, A. Azaro, M. Vieito, I. Brana, G. Mur, J. Ros, J. Mateos, G. Villacampa, R. Berche, M. Oliveira, M. Alsina, E. Elez, A. Oaknin, E. Munoz-Couselo, J. Carles, E. Felip, J. Rodon, J. Tabernero, R. Dienstmann, R. Perez-Lopez, E. Garralda, Capturing Hyperprogressive Disease with Immune-Checkpoint Inhibitors Using RECIST 1.1 Criteria. Clin Cancer Res 26, 1846-1855 (2020).
11. S. Hiratsuka, A. Watanabe, Y. Sakurai, S. Akashi-Takamura, S. Ishibashi, K. Miyake, M. Shibuya, S. Akira, H. Aburatani, Y. Maru, The S100A8-serum amyloid A3-TLR4 paracrine cascade establishes a pre-metastatic phase. Nat Cell Biol 10, 1349-1355 (2008).
12. S. Hiratsuka, S. Ishibashi, T. Tomita, A. Watanabe, S. Akashi-Takamura, M. Murakami, H. Kijima, K. Miyake, H. Aburatani, Y. Maru, Primary tumours modulate innate immune signalling to create pre-metastatic vascular hyperpermeability foci. Nat Commun 4, 1853 (2013).
13. A. Gast, J. L. Bermejo, R. Claus, A. Brandt, M. Weires, A. Weber, C. Plass, A. Sucker, K. Hemminki, D. Schadendorf, R. Kumar, Association of inherited variation in Toll-like receptor genes with malignant melanoma susceptibility and survival. PLoS One 6, e24370 (2011).
14. L. M. Booshehri, H. M. Hoffman, CAPS and NLRP3. J Clin Immunol 39, 277-286 (2019).
15. K. V. Swanson, M. Deng, J. P. Ting, The NLRP3 inflammasome: molecular activation and regulation to therapeutics. Nat Rev Immunol 19, 477-489 (2019).
16. M. Moossavi, N. Parsamanesh, A. Bahrami, S. L. Atkin, A. Sahebkar, Role of the NLRP3 inflammasome in cancer. Mol Cancer 17, 158 (2018).
17. Y. Wang, H. Kong, X. Zeng, W. Liu, Z. Wang, X. Yan, H. Wang, W. Xie, Activation of NLRP3 inflammasome enhances the proliferation and migration of A549 lung cancer cells. Oncol Rep 35, 2053-2064 (2016).
18. H. Wang, Q. Luo, X. Feng, R. Zhang, J. Li, F. Chen, NLRP3 promotes tumor growth and metastasis in human oral squamous cell carcinoma. BMC Cancer 18, 500 (2018).
19. B. Theivanthiran, K. S. Evans, N. C. DeVito, M. Plebanek, M. Sturdivant, L. P. Wachsmuth, A. K. Salama, Y. Kang, D. Hsu, J. M. Balko, D. B. Johnson, M. Starr, A. Nixon, A. Holtzhausen, B. A. Hanks, A tumor-intrinsic PD-L1/NLRP3 inflammasome signaling pathway drives resistance to anti-PD-1 immunotherapy. J Clin Invest 130, 2570-2586 (2020).
20. B. Theivanthiran, T. Haykal, L. Cao, A. Holtzhausen, M. Plebanek, N. C. DeVito, B. A. Hanks, Overcoming Immunotherapy Resistance by Targeting the Tumor-Intrinsic NLRP3-HSP70 Signaling Axis. Cancers (Basel) 13, (2021).
21. Y. Wang, Y. Ding, N. Guo, S. Wang, MDSCs: Key Criminals of Tumor Pre-metastatic Niche Formation. Front Immunol 10, 172 (2019).
22. M. E. Shaul, Z. G. Fridlender, Tumour-associated neutrophils in patients with cancer. Nat Rev Clin Oncol 16, 601-620 (2019).
23. V. Bronte, S. Brandau, S. H. Chen, M. P. Colombo, A. B. Frey, T. F. Greten, S. Mandruzzato, P. J. Murray, A. Ochoa, S. Ostrand-Rosenberg, P. C. Rodriguez, A. Sica, V. Umansky, R. H. Vonderheide, D. I. Gabrilovich, Recommendations for myeloid-derived suppressor cell nomenclature and characterization standards. Nat Commun 7, 12150 (2016).
24. J. Stackowicz, N. Gaudenzio, N. Serhan, E. Conde, O. Godon, T. Marichal, P. Starkl, B. Balbino, A. Roers, P. Bruhns, F. Jonsson, P. Moguelet, S. Georgin-Lavialle, L. Broderick, H. M. Hoffman, S. J. Galli, L. L. Reber, Neutrophil-specific gain-of-function mutations in Nlrp3 promote development of cryopyrin-associated periodic syndrome. J Exp Med 218, (2021).
25. S. D. Brydges, J. L. Mueller, M. D. McGeough, C. A. Pena, A. Misaghi, C. Gandhi, C. D. Putnam, D. L. Boyle, G. S. Firestein, A. A. Horner, P. Soroosh, W. T. Watford, J. J. O'Shea, D. L. Kastner, H. M. Hoffman, Inflammasome-mediated disease animal models reveal roles for innate but not adaptive immunity. Immunity 30, 875-887 (2009).
26. I. W. Tengesdal, D. R. Menon, D. G. Osborne, C. P. Neff, N. E. Powers, F. Gamboni, A. G. Mauro, A. D'Alessandro, D. Stefanoni, M. A. Henen, T. S. Mills, D. M. De Graaf, T. Azam, B. Vogeli, B. E. Palmer, E. M. Pietras, J. DeGregori, A. C. Tan, L. A. B. Joosten, M. Fujita, C. A. Dinarello, C. Marchetti, Targeting tumor-derived NLRP3 reduces melanoma progression by limiting MDSCs expansion. Proc Natl Acad Sci USA 118, (2021).
27. M. Okamoto, W. Liu, Y. Luo, A. Tanaka, X. Cai, D. A. Norris, C. A. Dinarello, M. Fujita, Constitutively active inflammasome in human melanoma cells mediating autoinflammation via caspase-1 processing and secretion of interleukin-1beta. J Biol Chem 285, 6477-6488 (2010).
28. H. Jia, C. P. Sodhi, Y. Yamaguchi, P. Lu, L. Y. Martin, M. Good, Q. Zhou, J. Sung, W. B. Fulton, D. F. Nino, T. Prindle, Jr., J. A. Ozolek, D. J. Hackam, Pulmonary Epithelial TLR4 Activation Leads to Lung Injury in Neonatal Necrotizing Enterocolitis. J Immunol 197, 859-871 (2016).
29. Y. S. Gui, L. Wang, X. Tian, R. Feng, A. Ma, B. Cai, H. Zhang, K. F. Xu, SPC-Cre-ERT2 transgenic mouse for temporal gene deletion in alveolar epithelial cells. PLoS One 7, e46076 (2012).
30. I. Omrane, O. Baroudi, N. Kourda, Y. J. Bignon, N. Uhrhammer, A. Desrichard, I. Medimegh, H. Ayari, N. Stambouli, A. Mezlini, H. Bouzayenne, R. Marrakchi, A. Benammar-Elgaaid, K. Bougatef, Positive link between variant Toll-like receptor 4 (Asp299Gly and Thr399Ile) and colorectal cancer patients with advanced stage and lymph node metastasis. Tumour Biol 35, 545-551 (2014).
31. L. Apetoh, F. Ghiringhelli, A. Tesniere, M. Obeid, C. Ortiz, A. Criollo, G. Mignot, M. C. Maiuri, E. Ullrich, P. Saulnier, H. Yang, S. Amigorena, B. Ryffel, F. J. Barrat, P. Saftig, F. Levi, R. Lidereau, C. Nogues, J. P. Mira, A. Chompret, V. Joulin, F. Clavel-Chapelon, J. Bourhis, F. Andre, S. Delaloge, T. Tursz, G. Kroemer, L. Zitvogel, Toll-like receptor 4-dependent contribution of the immune system to anticancer chemotherapy and radiotherapy. Nat Med 13, 1050-1059 (2007).
32. L. Kyjacova, R. Saup, M. Rothley, A. Schmaus, T. Wagner, A. Bosserhoff, B. K. Garvalov, W. Thiele, J. P. Sleeman, Quantitative Detection of Disseminated Melanoma Cells by Trp-1 Transcript Analysis Reveals Stochastic Distribution of Pulmonary Metastases. J Clin Med 10, (2021).
33. A. Ewens, E. Mihich, M. J. Ehrke, Distant metastasis from subcutaneously grown E0771 medullary breast adenocarcinoma. Anticancer Res 25, 3905-3915 (2005).
34. J. H. Cho, J. P. Robinson, R. A. Arave, W. J. Burnett, D. A. Kircher, G. Chen, M. A. Davies, A. H. Grossmann, M. W. VanBrocklin, M. McMahon, S. L. Holmen, AKT1 Activation Promotes Development of Melanoma Metastases. Cell Rep 13, 898-905 (2015).
35. R. N. Kaplan, R. D. Riba, S. Zacharoulis, A. H. Bramley, L. Vincent, C. Costa, D. D. MacDonald, D. K. Jin, K. Shido, S. A. Kerns, Z. Zhu, D. Hicklin, Y. Wu, J. L. Port, N. Altorki, E. R. Port, D. Ruggero, S. V. Shmelkov, K. K. Jensen, S. Rafii, D. Lyden, VEGFR1-positive haematopoietic bone marrow progenitors initiate the pre-metastatic niche. Nature 438, 820-827 (2005).
36. A. Holtzhausen, F. Zhao, K. Evans, M. Tsutsui, C. Orabona, D. S. Tyler, B. A. Hanks, Melanoma-derived Wnt5a Promotes Local Dendritic-Cell Expression of IDO and Immunotolerance: Opportunities for Pharmacologic Enhancement of Immunotherapy. Cancer Immunol Res 3, 1082-1095 (2015).
37. F. Zhao, K. Evans, C. Xiao, N. DeVito, B. Theivanthiran, A. Holtzhausen, P. J. Siska, G. Blobe, B. A. Hanks, Stromal Fibroblasts Mediate Anti-PD-1 Antibody Resistance via MMP-9 and Dictate TGF-β Inhibitor Therapy Sequencing in Melanoma. Cancer Immunology Research 6, (2018).
38. Z. Kan, B. S. Jaiswal, J. Stinson, V. Janakiraman, D. Bhatt, H. M. Stern, P. Yue, P. M. Haverty, R. Bourgon, J. Zheng, M. Moorhead, S. Chaudhuri, L. P. Tomsho, B. A. Peters, K. Pujara, S. Cordes, D. P. Davis, V. E. Carlton, W. Yuan, L. Li, W. Wang, C. Eigenbrot, J. S. Kaminker, D. A. Eberhard, P. Waring, S. C. Schuster, Z. Modrusan, Z. Zhang, D. Stokoe, F. J. de Sauvage, M. Faham, S. Seshagiri, Diverse somatic mutation patterns and pathway alterations in human cancers. Nature 466, 869-873 (2010).
39. J. L. Benci, B. Xu, Y. Qiu, T. J. Wu, H. Dada, C. Twyman-Saint Victor, L. Cucolo, D. S. M. Lee, K. E. Pauken, A. C. Huang, T. C. Gangadhar, R. K. Amaravadi, L. M. Schuchter, M. D. Feldman, H. Ishwaran, R. H. Vonderheide, A. Maity, E. J. Wherry, A. J. Minn, Tumor Interferon Signaling Regulates a Multigenic Resistance Program to Immune Checkpoint Blockade. Cell 167, 1540-1554 e1512 (2016).
40. S. Koyama, E. Sato, T. Masubuchi, A. Takamizawa, K. Kubo, S. Nagai, T. Izumi, Alveolar type II-like cells release G-CSF as neutrophil chemotactic activity. Am J Physiol 275, L687-693 (1998).
41. Y. M. Kim, H. Kim, S. Lee, S. Kim, J. U. Lee, Y. Choi, H. W. Park, G. You, H. Kang, S. Lee, J. S. Park, Y. Park, H. S. Park, C. S. Park, S. W. Lee, Airway G-CSF identifies neutrophilic inflammation and contributes to asthma progression. Eur Respir J 55, (2020).
42. L. Strauss, M. A. A. Mahmoud, J. D. Weaver, N. M. Tij aro-Ovalle, A. Christofides, Q. Wang, R. Pal, M. Yuan, J. Asara, N. Patsoukis, V. A. Boussiotis, Targeted deletion of PD-1 in myeloid cells induces antitumor immunity. Sci Immunol 5, (2020).
43. M. S. Alam, Proximity Ligation Assay (PLA). Curr Protoc Immunol 123, e58 (2018).
44. R. Varnier, T. Garrivier, E. Hafliger, A. Favre, C. Coutzac, C. Spire, P. Rochefort, M. Sarabi, F. Desseigne, P. Guibert, A. Cattey-Javouhey, P. Funk-Debleds, C. Mastier, A. Buisson, D. Perol, O. Tredan, J. Y. Blay, J. M. Phelip, C. de la Fouchardiere, Hyperprogressive Disease After Combined Anti-PD-L1 and Anti-CTLA-4 Immunotherapy for MSI-H/dMMR Gastric Cancer: A Case Report. Front Oncol 11, 756365 (2021).
45. Y. Liu, Y. Gu, Y. Han, Q. Zhang, Z. Jiang, X. Zhang, B. Huang, X. Xu, J. Zheng, X. Cao, Tumor Exosomal RNAs Promote Lung Pre-metastatic Niche Formation by Activating Alveolar Epithelial TLR3 to Recruit Neutrophils. Cancer Cell 30, 243-256 (2016).
46. F. R. Di Pietro, S. Verkhovskaia, S. Mastroeni, M. L. Carbone, D. Abeni, C. Z. Di Rocco, N. Sama, A. R. Zappala, P. Marchetti, F. De Galitiis, C. M. Failla, C. Fortes, Clinical Predictors of Response to Anti-PD-1 First-Line Treatment in a Single-Centre Patient Cohort: A Real-World Study. Clin Oncol (R Coll Radiol) 34, e18-e24 (2022).
47. K. Kumawat, M. H. Menzen, I. S. Bos, H. A. Baarsma, P. Borger, M. Roth, M. Tamm, A. J. Halayko, M. Simoons, A. Prins, D. S. Postma, M. Schmidt, R. Gosens, Noncanonical WNT-5A signaling regulates TGF-beta-induced extracellular matrix production by airway smooth muscle cells. FASEB J 27, 1631-1643 (2013).
48. I. W. Tengesdal, A. Dinarello, N. E. Powers, M. A. Burchill, L. A. B. Joosten, C. Marchetti, C. A. Dinarello, Tumor NLRP3-Derived IL-1beta Drives the IL-6/STAT3 Axis Resulting in Sustained MDSC-Mediated Immunosuppression. Front Immunol 12, 661323 (2021).
49. Q. Liang, J. Wu, X. Zhao, S. Shen, C. Zhu, T. Liu, X. Cui, L. Chen, C. Wei, P. Cheng, W. Cheng, A. Wu, Establishment of tumor inflammasome clusters with distinct immunogenomic landscape aids immunotherapy. Theranostics 11, 9884-9903 (2021).
50. S. Angelicola, F. Ruzzi, L. Landuzzi, L. Scalambra, F. Gelsomino, A. Ardizzoni, P. Nanni, P. L. Lollini, A. Palladini, IFN-gamma and CD38 in Hyperprogressive Cancer Development. Cancers (Basel) 13, (2021).
51. D. S. Sohal, M. Nghiem, M. A. Crackower, S. A. Witt, T. R. Kimball, K. M. Tymitz, J. M. Penninger, J. D. Molkentin, Temporally regulated and tissue-specific gene manipulations in the adult and embryonic heart using a tamoxifen-inducible Cre protein. Circ Res 89, 20-25 (2001).
52. F. Zhao, C. Xiao, K. S. Evans, T. Theivanthiran, N. DeVito, A. Holtzhausen, J. Liu, X. Liu, D. Boczkowski, S. Nair, J. W. Locasale, B. A. Hanks, Paracrine Wnt5a-beta-Catenin Signaling Triggers a Metabolic Program that Drives Dendritic Cell Tolerization. Immunity 48, 147-160 e147 (2018).
53. B. A. Pulaski, S. Ostrand-Rosenberg, Mouse 4T1 breast tumor model. Curr Protoc Immunol Chapter 20, Unit 20 22 (2001).
54. L. S. Qi, M. H. Larson, L. A. Gilbert, J. A. Doudna, J. S. Weissman, A. P. Arkin, W. A. Lim, Repurposing CRISPR as an RNA-guided platform for sequence-specific control of gene expression. Cell 152, 1173-1183 (2013).
55. J. Joung, P. C. Kirchgatterer, A. Singh, J. H. Cho, S. P. Nety, R. C. Larson, R. K. Macrae, R. Deasy, Y. Y. Tseng, M. V. Maus, F. Zhang, CRISPR activation screen identifies BCL-2 proteins and B3GNT2 as drivers of cancer resistance to T cell-mediated cytotoxicity. Nat Commun 13, 1606 (2022).

### Example 2—Activity of the Tumor-Intrinsic NLRP3 Inflammasome Pathway Predicts for Response to Checkpoint Inhibitor Immunotherapy in Melanoma Patients

Reference is made to the abstract: Haykal et al., “Activity of the Tumor-intrinsic NLRP3 Inflammasome Pathway Predicts for Response to Checkpoint Inhibitor Immunotherapy in Melanoma Patients,” the content of which is incorporated herein by reference in its entirety.

Background: We have previously determined that activation of a novel tumor-intrinsic NOD-, LRR- and pyrin domain-containing protein-3 (NLRP3) inflammasome-heat shock protein-70 (HSP70) signaling axis in response to PD-1 blockade triggers the recruitment of granulocytic myeloid-derived suppressor cells (PMN-MDSCs) into the tumor microenvironment, suppresses anti-tumor immunity and, in select settings, promotes tumor hyperprogression. We, therefore, sought to determine whether the activity of the tumor-intrinsic NLRP3-HSP70 pathway may correlate with anti-PD-1 response by interrogating clinical specimens derived from advanced melanoma patients undergoing anti-PD-1 monotherapy.
Methods: Three independent approaches were utilized to measure the activity of the tumor-intrinsic NLRP3-HSP70 signaling pathway in 60 advanced melanoma patients undergoing either pembrolizumab or nivolumab monotherapy: 1. baseline week 0 plasma HSP70 levels were measured by ELISA, 2. germline PCR-based genotyping was performed to detect the single-nucleotide polymorphism (SNP), rs12239046, previously associated with enhanced NLRP3 expression, 3. PCR-based proximity ligation assay (PLA) analysis targeting the NLRP3-ASC proteins in baseline formalin-fixed paraffin-embedded tumor tissue specimens. Detection of the rs12239046 SNP was correlated with progression-free survival (PFS) while plasma HSP70 and NLRP3-ASC PLA levels were correlated with objective response (OR) based on RECIST1.1 assessment of week-12 CT imaging as well as PFS and overall survival (OS).
Results: Our studies demonstrate that elevated baseline plasma HSP70 levels (P=0.0008) and elevated baseline tissue NLRP3-ASC PLA levels (P=0.0014) independently correlate with resistance to anti-PD-1 immunotherapy (ICI) based on week-12 OR in melanoma patients. Importantly, melanoma patients developing disease hyperprogression in response to ICI exhibited elevations in baseline plasma HSP70 levels (P=<0.0001) and baseline tissue NLRP3-ASC PLA levels (P=<0.0001) relative to patients with week-12 disease progression. Above median baseline tissue NLRP3-ASC PLA levels were determined to correlate with both inferior PFS (HR 0.12, P=0.0008) and OS (HR 0.16, P=0.0456) in advanced melanoma patients undergoing ICI. Germline PCR detection of the rs12239046 SNP was found to be associated with elevated plasma HSP70 levels and trended toward a correlation with inferior PFS (HR 0.50, P=0.07).
Conclusion: Baseline markers of the tumor-intrinsic NLRP3-HSP70 signaling pathway correlate with resistance and disease hyperprogression in melanoma patients undergoing anti-PD-1 immunotherapy. These data strongly support the important role of the tumor-intrinsic NLRP3 inflammasome in regulating responses to anti-PD-1 therapy and verify its relevance as a pharmacologic target to enhance immunotherapy efficacy. Expanded studies are warranted to confirm these findings in a larger patient cohort.

1. A method for treating cancer in a subject selected for responsiveness to the treatment, comprising:
a. obtaining a biological sample from the subject,
b. determining the level or activity of a biomarker in the biological sample, wherein the biomarker comprises markers of activation of the NLRP3-HSP70 axis,
c. comparing the level or activity of the biomarker to a control,
d. classifying the subject for likelihood of clinical response to anti-cancer immunotherapy, wherein the levels of the biomarker correlates with anti-cancer immunotherapy efficacy; and
e. administering anti-cancer immunotherapy to the subject wherein the level of the biomarker indicates the subject is likely to be responsive to the anti-cancer immunotherapy or administering an anti-cancer therapy other than immunotherapy wherein the level of the biomarker indicates the subject is unlikely to be responsive to the anti-cancer immunotherapy.

2. The method of claim 1, wherein the level of biomarker indicates that the subject is likely to be responsive and step e) comprises administering the anti-cancer immunotherapy to the subject.

3. The method of claim 1, wherein the level of biomarker indicates that the subject is unlikely to be responsive and step e) comprises administering an anti-cancer therapy other than immunotherapy.

4. The method of claim 1, wherein the subject is evaluated for the development of disease hyperprogression.

5. The method of claim 1 wherein a marker of activation of the NLRP3-HSP70 axis comprises HSP70.

6. The method of claim 5 wherein a marker of activation of the NLRP3-HSP70 axis further comprises, NLRP3, NLRP3 activity, NLRP3-ACS proximal ligation assay or SEQ ID NO: 1

7. The method of claim 6, wherein the level or activity of the biomarker is determined using Q-RT-PCR, Western blot, RNA sequencing, proteomic studies, sequencing, fluorescent in situ hybridization (FISH), ELISA, immunostaining, or proximal ligation assay.

8. A method of claim 1, wherein the subject has cancer, wherein the cancer comprises melanoma, breast cancer, renal cell carcinoma, non-small cell lung cancer, colorectal cancer, Merkel cell carcinoma, gastroesophageal cancer, gastric cancer or pancreatic cancer.

9. The method of claim 1, wherein the biological sample is tumor tissue, plasma, serum, blood, tissue or peripheral blood mononuclear cells.

10. The method of claim 9, wherein the biological sample comprises serum or plasma and a marker of activation of the NLRP3-HSP70 axis comprises HSP70.

11. The method of claim 1 where in the anti-cancer immunotherapy comprises immune blockade therapy or immune an immune checkpoint inhibitor.

12. The method of claim 11, where in the immune checkpoint inhibitor comprises a PD-1 inhibitor or a PD-L1 inhibitor.

13. The method of claim 1, wherein the method further comprises administering NLRP3 inhibitor.

14. The method of claim 13, wherein the NLRP3 inhibitor comprises a small molecule inhibitor.

15. The method of claim 1, wherein the biomarker is measured prior to any anti-cancer treatment.

16. The method of claim 1, wherein the biomarker is measured after beginning an anti-cancer therapy.

17. The method of claim 1, wherein the biomarker is measured more than once.

18. The method of claim 1, wherein the subject has a diagnosis of stage 3 or stage 4 cancer.

19. A method of treating a subject undergoing anti-cancer immunotherapy, the method comprising:
a. obtaining a biological sample from the subject,
b. determining the level or activity of a biomarker in the biological sample, wherein the biomarker comprises markers of activation of the NLRP3-HSP70 axis,
c. comparing the level or activity of the biomarker to a control and
d. ceasing the administration of the anti-cancer immunotherapy if the level or activity of the biomarker is greater than the control.

20. A method of treating a subject who is refractory or not responding to immune checkpoint inhibitor therapy, the method comprising:
a. obtaining a biological sample from the subject,
b. determining the level or activity of a biomarker in the biological sample, wherein the biomarker comprises markers of activation of the NLRP3-HSP70 axis,
c. comparing the level or activity of the biomarker to a control,
d. administering an anti-cancer immunotherapy treatment to the subject if the level or activity of the biomarker is lower than that of the control in step (c) or not administering an anti-cancer immunotherapy to the subject if the biomarker is higher than the level in the control sample of step (c).
